(19) United States
(12) Patent Application Publication
(10) Pub. No.: US 2008/0159970 A1
(43) Pub. Date: Jul. 3, 2008

(54) KIT COMPRISING SILICONE COMPOUNDS AND A COSMETIC AND/OR DERMATOLOGICAL ACTIVE AGENT

(75) Inventor: Claudie Willemin, Paris (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, Paris (FR)

(21) Appl. No.: 11/961,031

(22) Filed: Dec. 20, 2007

Related U.S. Application Data
(60) Provisional application No. 60/870,915, filed on Dec. 20, 2006.

(51) Int. Cl. A61K 8/89; A61K 47/30; A61Q 1/02; A61Q 3/02; A61Q 5/00; A61Q 19/00; A61Q 17/04

(52) U.S. Cl. 424/59; 514/772.4; 424/64; 424/61; 424/70.12; 424/63

(57) ABSTRACT

Kit for making up or non-therapeutically caring for keratin material(s), containing
at least 0.01% by weight relative to the total weight of the composition of a moisturizer agent for improving the appearance of the keratin materials,
at least two different compositions conditioned separately, the kit comprising at least one compound X and at least one compound Y, and at least one catalyst, with at least one of the compounds X and Y being a silicone compound, and the compounds X and Y being capable of reacting together via a hydrosilylation reaction in the presence of a catalyst, when they are placed in contact with each other,
   the moisturizer agent(s) being present in at least one of the two first compositions, or in a third composition separate from the first two.

### REFERENCE TO PRIOR APPLICATIONS

This application claims priority to U.S. provisional application 60/870,915 filed Dec. 20, 2006, incorporated herein by reference.

### FIELD OF THE INVENTION

The field of the invention especially concerned is the formulation of cosmetic and/or dermatological active agents, in particular a moisturizer agent, for improving the appearance of keratin materials, in particular the skin, with silicone compounds capable of forming in situ a polymer film on the keratin materials, so as to increase their availability and/or activity after application to the keratin materials.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### DETAILED DESCRIPTION OF THE PREFERRED EMBODIMENTS

The expression “improving the appearance of keratin materials” means improving the aesthetic appearance of the materials, especially reducing the surface irregularities of the keratin materials and/or improving the texture and/or the mechanical properties of the keratin materials.

One subject of the present invention is mainly a cosmetic and/or dermatological kit for making up or non-therapeutically caring for keratin material(s), comprising
at least two compositions and containing at least one compound X and at least one compound Y, compounds X and Y being capable of reacting together if necessary in the presence of a catalyst or of a peroxide and at least one of the compounds being a silicone compound, and
at least one cosmetic and/or dermatological active agent for improving the appearance of the keratin materials,
   the cosmetic and/or dermatological active agent(s) being present in at least one of the two first compositions, or in a third composition separate from the first two.

According to a first embodiment, the cosmetic and/or dermatological active agent(s) as defined later in the description is (are) present in the composition containing at least one compound X.

According to another embodiment, the cosmetic and/or dermatological active agent(s) as defined later in the description is (are) present in the composition containing at least one compound Y.

According to another embodiment, the cosmetic and/or dermatological active agent(s) as defined later in the description is (are) present in the two compositions containing, respectively, a compound X or a compound Y.

According to yet another embodiment, the cosmetic and/or dermatological active agent(s) as defined later in the description is (are) present in a third composition separate from the first two.

The kits according to the invention may be makeup or care products for keratin materials, in particular for the skin, the lips, the eyelashes, the eyebrows or the nails.

More specifically, the makeup products may be of the type such as foundations, makeup rouges, eyeshadows, concealer products, blushers, lipsticks, lip balms, lip glosses, mascaras, eyeliners, body makeup products or skin colouring products.

The skincare products may be a protective, treating or care composition for the face, for the hands, for the feet, for the major anatomical folds or for the body (for example the day cream, night cream, makeup-removing cream, antisun composition, protective or care body milk, after-sun milk, skincare lotion, gel or mousse, or artificial tanning composition); an aftershave composition.

The present invention is more particularly directed towards proposing a novel method for formulating cosmetic and/or dermatological active agents, making it possible to obtain a film deposited on the keratin materials that has good cosmetic properties, especially in terms of staying power and matting effect, and forming a comfortable deposit on the skin, the film promoting better distribution and/or availability of the active agents according to the invention and the surface of the keratin materials and/or better activity after application of the compositions to the keratin materials.

Recently, the inventors have found that it is possible to obtain such properties in terms of cosmetically and biological activity by exploiting, in combination with cosmetic and/or dermatological active agents for improving the appearance of keratin materials, the capacity of certain compounds, especially silicone compounds, to interact when they are placed in contact and to constitute after their interaction a polymer film.

In particular, these silicone compounds improve and/or increase the moisturizing properties of a moisturizer, when they are used together with such a moisturizer agent according to the invention.

Thus, compounds referred to as compound X and compound Y, as defined below, prove to be capable if necessary in the presence of a catalyst or of a peroxide of polymerizing in situ, at atmospheric pressure and room temperature and of forming films that are advantageously biocompatible, non-tacky, slightly opalescent, or even peelable. Such systems are especially partly described in patents WO 01/96450 and GB 2 407 496 from Dow Corning.

These polymer films that may be formed in situ on a support, especially of the keratin material type, turn out to have advantageous properties in cosmetic terms, namely good adhesion, good staying power and comfort, and afford the cosmetic and/or dermatological active agents used in this system improved availability and/or efficacy at the site of application of the compositions.

Thus, according to a first aspect, one subject of the present invention is a cosmetic and/or dermatological kit comprising:
at least two different compositions conditioned separately, the kit comprising at least one compound X, at least one compound Y, and optionally at least one catalyst or one peroxide, with at least one of the compounds X or Y being a silicone compound and the compounds X and Y being capable of reacting together via a hydrosilylation reaction in the presence of a catalyst, or via a condensation reaction, or via a crosslinking reaction in the presence of a peroxide, when they are placed in contact with each other if necessary in the presence of a catalyst or of a peroxide, and
   at least one cosmetic and/or dermatological active agent for improving the appearance of keratin materials, the cosmetic and/or dermatological active agent(s) being present in at least one of the first two compositions or in a third composition separate from the first two compositions.

Preferably, the active agent(s) will advantageously be in at least one of the two compositions containing, respectively, compound X or compound Y.

According to one particular mode, the kit is such that compounds X and Y and the catalyst or the peroxide, when they are present, are not simultaneously present in the same composition.

According to one preferred mode, at least one of the compositions of the kit is in the form of a simple or W/O/W multiple emulsion in which the compound X or Y is present in the oily phase. This galenical form advantageously makes it possible to reduce the sheen of the films obtained by reacting the compounds X and Y, reduced sheen of the films being especially desired for foundation products and/or skincare products.

When the cosmetic and/or dermatological active agent(s) is (are) present in one or the other of the compositions in the form of a simple or W/O/W multiple emulsion containing, respectively, compound X or compound Y, the active agent(s) will be formulated in the oily phase or the aqueous phase of the emulsion as a function of their hydrophilic and/or hydrophobic nature.

The compound(s) X and the compound(s) Y may be applied to the keratin materials using several compositions containing compound(s) X and compound(s) Y, alone or as a mixture, or from a single composition containing compound(s) X and compound(s) Y.

According to a first embodiment variant, the kit comprises:
at least
   i. a first composition containing, in a physiologically acceptable medium, at least one compound X, and
   ii. a second composition containing, in a physiologically acceptable medium, at least one compound Y, and at least one of said first and second compositions additionally containing if necessary at least one catalyst or a peroxide, and
at least one cosmetic and/or dermatological active agent as defined below, the active agent(s) being present in at least one of the first or second compositions, or in a third composition separate from the first two.

Preferentially, the cosmetic active is a moisturizer agent, which represents at least 0.01% by weight, preferably at least 0.1% by weight relative to the total weight of the composition.

According to a first embodiment, the cosmetic and/or dermatological active agent(s) as defined later in the description is (are) present in the first composition containing at least one compound X.

According to another embodiment, the cosmetic and/or dermatological active agent(s) as defined later in the description is (are) present in the second composition containing at least one compound Y.

According to another embodiment, the cosmetic and/or dermatological active agent(s) as defined later in the description is (are) present in the two compositions containing, respectively, a compound X and a compound Y.

According to yet another embodiment, the cosmetic and/or dermatological active agent(s) as defined later in the description is (are) present in a third composition separate from the two first compositions.

The first and second compositions are different from each other.

For example, the first composition is advantageously free of compound Y and the second composition is advantageously free of compound X. Specifically, with regard to their high reactivity with each other, compounds X and Y are not simultaneously present in a first and/or second composition forming a kit according to the invention when their interaction is not conditioned by the presence of a catalyst or a peroxide.

According to a second embodiment variant, the kit comprises:
at least:
i. a first composition containing, in a physiologically acceptable medium, at least one compound X and one compound Y, the compounds X and Y being capable of reacting together in the presence of a catalyst via a hydrosilylation reaction, or via a condensation reaction, or via a crosslinking reaction in the presence of a peroxide, when they are placed in contact with each other, and
ii. a second composition containing, in a physiologically acceptable medium, at least the catalyst required for the interaction of compounds X and Y,
at least one cosmetic and/or dermatological active agent as defined below, the active agent(s) being present in at least one of the first or second compositions, or in a third composition separate from the first two.

According to a first embodiment, the cosmetic and/or dermatological active agent(s) as defined later in the description is (are) present in the first composition containing at least one compound X and one compound Y.

According to another embodiment, the cosmetic and/or dermatological active agent(s) as defined later in the description is (are) present in the second composition containing the catalyst.

According to yet another embodiment, the cosmetic and/or dermatological active agent(s) as defined later in the description is (are) present in the first two compositions.

According to yet another embodiment, the cosmetic and/or dermatological active agent(s) as defined later in the description is (are) present in a third composition separate from the first two.

In the sense of the invention, notably in the embodiment where the composition is obtained as described above, namely by mixing, at the time of use, a first composition containing at least compound X and a second composition containing at least compound Y, it is to be understood that the mixture thus formed comprises compounds X and/or Y in a form that has not yet reacted and not exclusively in the form of their reaction product by hydrosilylation, by polycondensation and/or by crosslinking in the presence of a peroxide.

Thus, formation of the reaction product according to the invention can either be carried out directly on the surface of the keratinous substance that is to be treated, or initiated just before application by extemporaneous mixing of compounds X and Y in conditions favourable for their interaction, formation of the reaction product being in the latter case finalized on the surface of the keratinous substance.

For obvious reasons, and in view of the great reactivity of compounds X and/or Y, it is in fact necessary that their application should be carried out in conditions that are favourable for the manageability of the composition containing it (or them) notably with respect to its spreading, for example. The method according to the invention therefore employs a composition containing compounds X and Y, and therefore not congealed in the form of the expected final film resulting from reaction of all of X and/or of all of Y.

In particular, the catalyst is formulated in an aqueous phase.

A person skilled in the art will know how to select, as a function of the nature and the solubility of the cosmetic and/or dermatological active agents, the embodiment that is most suitable.

When the active agent is lipophilic, it will advantageously be present in an oily phase of a composition, this phase possibly being one and/or the other of the compositions with compounds X or Y, or another composition separate from these compositions.

When the active agent is hydrophilic, it will advantageously be present in an aqueous phase of a composition, this composition possibly being either one and/or the other of the compositions containing the compounds X or Y and being in the form of a simple or W/O/W emulsion, or the composition containing the catalyst, or alternatively another composition separate from these compositions.

A person skilled in the art will be able to choose from the lists of active agents mentioned above, and as a function of his general knowledge regarding their hydrophilic/lipophilic nature, the suitable modes of preparation.

According to one particular mode, the compositions contain at least one compound X or one compound Y in the form of a simple or W/O/W multiple emulsion in which compounds X and Y are present in the oily phase.

Advantageously, the composition(s) in emulsion form of the kits according to the invention is (are) oil-in water direct emulsions.

Preferably, the compositions of the kit and in particular the first composition comprising compound X and the second composition comprising compound Y of the kit are conditioned in separate conditioning.

For example, each composition may be conditioned separately in the same conditioning article, for example in a two-compartment or three-compartment pen, the base composition being delivered by one end of the pen and the top composition being delivered by the other end of the pen, each end being closed, especially in a leaktight manner, by a cap. Each composition may also be conditioned in a compartment within the same conditioning article, the mixing of the compositions of the kit then taken place at the end(s) of the conditioning article during the delivery of each composition.

Alternatively, each of the first and second compositions may be conditioned in a different conditioning article.

This particular mode especially allows a standard application of the compositions of the kit according to the invention: it is possible, for example, to apply the composition containing the active agent(s) and subsequently to apply compositions containing, respectively, compound X or compound Y intended to form in situ a polymer film on the keratin materials.

The invention also relates to a cosmetic care and/or makeup process for keratin material(s), comprising at least the application (a) of at least one cosmetic active agent as defined below, (b) of one or more compounds X, (c) of one or more compounds Y with at least one of the compounds X and Y being a silicone compound and the compounds X and Y being capable of reacting together via a hydrosilylation reaction in the presence of a catalyst or via a condensation reaction, or via a crosslinking reaction in the presence of a peroxide, when they are placed in contact with each other, and, where appropriate, (d) of a catalyst or a peroxide if necessary for the interaction of the compound X with the compound Y, the applications of (a), (b), (c) and (d) possibly being simultaneous or sequential in any order, with the proviso that it is beneficial to the interaction of the compounds X and Y.

Thus, the cosmetic active agent, compound(s) X and compound(s) Y may be applied to the keratin materials using several compositions, the compositions containing, respectively, compound(s) X, compound(s) Y and the active agent(s), alone or as a mixture, or using a single composition containing compound(s) X and compound(s) Y and the active agent(s).

According to one particular embodiment of the invention, a first composition comprising at least compound(s) X, and a second composition containing at least compound(s) Y are applied to the keratin materials, with at least one of the first and second compositions comprising at least one cosmetic active agent and if necessary at least one catalyst or peroxide.

Several coats of each of the first and second compositions may also be applied alternately to the keratin materials.

The applied composition may also be obtained by extemporaneously mixing a first composition containing at least compound X and a second composition containing at least compound Y at least one of the first and second compositions also comprising at least one cosmetic and/or dermatological active agent.

As stated above, the compositions are advantageously in the form of a simple emulsion.

According to another embodiment, the compositions are in the form of a W/O/W multiple emulsion.

According to one embodiment, at least one additional coat of at least one third composition comprising a cosmetically acceptable medium, and preferably at least one film-forming a polymer and at least one organic (or oily) or aqueous solvent medium, is applied onto the coat(s) of the composition(s) according to the invention comprising compounds X and Y in order, for example, to improve the staying power and/or the comfort thereof.

According to a second aspect of the invention, compounds X and Y are formulated in a single composition, with at least one of the compounds being in an encapsulated form. Therefore, according to a second aspect, the kit according to the invention comprises:
a single composition containing compounds X and Y, if necessary at least one catalyst or peroxide, with at least one of the compounds X and Y being in an encapsulated form, and
at least one cosmetic and/or dermatological active agent for improving the appearance of keratin materials,
   the cosmetic and/or dermatological active agent(s) being in the single composition containing compounds X and Y, or in a composition that is separate therefrom.

According to one particular mode, the cosmetic and/or dermatological active agent(s) may itself (themselves) be encapsulated. This mode may be advantageous in the event of incompatibility of the active agent with one or the other of the compounds X and Y or of incompatibility with the oily phase in which they must be formulated.

Thus, the present invention is also directed towards a cosmetic and/or dermatological composition especially for caring for and/or making up keratin material(s) containing, in a physiologically acceptable medium:
at least one compound X and one compound Y as defined above, if necessary with at least one catalyst or peroxide, with at least one of the compounds X and Y being in an encapsulated form, and
at least one cosmetic and/or dermatological active agent.

According to one preferred embodiment variant, the two compounds X and Y are present in separate encapsulated forms.

According to this embodiment, the two compounds X and Y may be conditioned in the same composition while at the same time obviating the risk of premature reaction between them. This reaction takes place only when the composition is handled beforehand or when it is applied to the keratin material. The encapsulated form(s) and compounds X and Y may react to form the expected film.

### Compounds X and Y

The term “silicone compound” means a polyorganosiloxane compound, i.e. compound comprising at least two organosiloxane units, for example at least 5 organosiloxane units, notably at least 10 organosiloxane units. According to one particular embodiment, at least one of compounds X and Y, or compounds X and compounds Y are silicone compounds. Compounds X and Y may be aminated or non-aminated.

According to another embodiment, at least one of the compounds X and Y is a polymer whose main chain is predominantly formed from organosiloxane units.

Among the silicone compounds mentioned below, some may simultaneously have film-forming properties and adhesive properties, depending, for example, on the silicone proportion thereof or on whether they are used as a mixture with a particular additive. It is consequently possible to modify the film-forming properties or the adhesive properties of such compounds according to the intended use, and this is in particular the case for reactive silicone elastomers known as “room-temperature-vulcanizable” elastomers.

Compounds X and Y may react together at a temperature ranging between room temperature and 180° C. Advantageously, compounds X and Y can react together at room temperature (20±5° C.) and atmospheric pressure, or advantageously in the presence of a catalyst, via a hydrosilylation reaction or a condensation reaction, or a crosslinking reaction in the presence of a peroxide.

### Polar Groups

According to one particular embodiment, at least one of the compounds X and Y, for example compound X, bears at least one polar group capable of forming at least one hydrogen bond with keratin materials.

The term “polar group” means a group comprising carbon and hydrogen atoms in its chemical structure and at least one heteroatom (such as O, N, S and P), such that the group is capable of establishing at least one hydrogen bond with keratin materials.

Compounds bearing at least one group capable of establishing hydrogen bonds are particularly advantageous, since they afford the compositions containing them better adhesion to keratin materials.

The polar group(s) borne by at least one of the compounds X and Y is (are) capable of establishing a hydrogen bond, and comprise(s) either a hydrogen atom bonded to an electronegative atom, or an electronegative atom, for instance an oxygen, nitrogen or sulfur atom. When the group comprises a hydrogen atom bonded to an electronegative atom, the hydrogen atom may interact with another electronegative atom borne, for example, by another molecule, such as keratin, to form a hydrogen bond. When the group comprises an electronegative atom, the electronegative atom may interact with a hydrogen atom bonded to an electronegative atom borne, for example, by another molecule, such as keratin, to form a hydrogen bond.

Advantageously, these polar groups may be chosen from the following groups:
carboxylic acids —COOH,
alcohols, such as: —CH₂OH or —CH(R)OH, R being an alkyl radical containing from 1 to 6 carbon atoms,
amino of formula —NR₁R₂, in which R₁ and R₂, which may be identical or different, represent an alkyl radical containing from 1 to 6 carbon atoms, or R₁ or R₂ denotes a hydrogen atom, and the other of R₁ and R₂ represents an alkyl radical containing from 1 to 6 carbon atoms,
pyridino,
amido of formula —NH—COR′ or —CO—NH—R′ in which R′ represents a hydrogen atom or an alkyl radical containing from 1 to 6 carbon atoms,
pyrrolidino preferably chosen from the groups of formula:

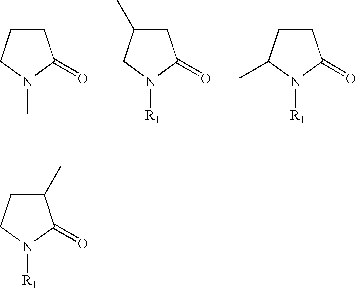

R₁ being an alkyl radical containing from 1 to 6 carbon atoms,
carbamoyl of formula —O—CO—NH—R′ or —NH—CO—OR′, R′ being as defined above,
thiocarbamoyl such as —O—CS—NH—R′ or —NH—CS—OR′, R′ being as defined above,
ureyl such as —NR′-CO—N(R′)₂, the groups R′, which may be identical or different, being as defined above,
sulfonamido such as —NR′-S(═O)₂—R′, R′ corresponding to the above definition.

Preferably, these polar groups are present in a content of less than or equal to 10% by weight, preferably less than or equal to 5% by weight, for example in a content ranging from 1% to 3% by weight, relative to the weight of each compound X or Y.

The polar group(s) may be located in the main chain of compound X and/or Y or may be pendent on the main chain or located at the ends of the main chain of compound X and/or Y.

### 1—Compounds X and Y Capable of Reacting via Hydro-Silylation

According to one embodiment, the invention relates to a cosmetic kit that can be used for the care and/or make-up of keratinous substance(s), notably of the skin, comprising at least two compositions that are different and are packaged separately with at least one of the compositions being of the emulsion type and comprising at least one cosmetic or dermatologic active, the kit comprising one or more compounds X, one or more compounds Y, and at least one catalyst, with at least one of compounds X and Y being a silicone compound and said compounds X and Y being capable of reacting together by a hydrosilylation reaction when they are brought into contact with one another in the presence of a catalyst, and in which the compounds X, Y and the catalyst are not present simultaneously in the same composition.

In particular, the cosmetic active is a moisturizer agent which represents at least 0.1% by weight, preferably at least 0.1% by weight relative to the total weight of the composition.

According to this embodiment, compounds X and Y are capable of reacting via hydrosilylation in the presence of a catalyst, this reaction being represented schematically in simple terms as follows:

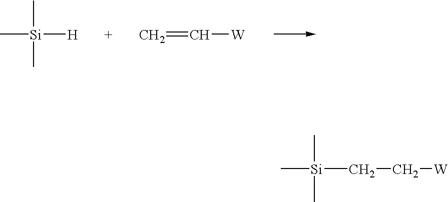

with W representing a carbon-based and/or silicone chain containing one or more unsaturated aliphatic groups.

In this case, compound X may be chosen from silicone compounds comprising at least two unsaturated aliphatic groups. For example, compound X may be a polyorganosiloxane comprising a silicone main chain whose unsaturated aliphatic groups are pendent on the main chain (side group) or located at the ends of the main chain of the compound (end group). In the rest of the description, these particular compounds will be referred to as polyorganosiloxanes containing unsaturated aliphatic groups.

According to one embodiment, compound X and/or compound Y bears at least one polar group, as described above, capable of forming at least one hydrogen bond with keratin materials. This polar group is advantageously borne by compound X that comprises at least two unsaturated aliphatic groups.

According to one embodiment, compound X is chosen from polyorganosiloxanes comprising at least two unsaturated aliphatic groups, for example two or three vinyl or allylic groups, each bonded to a silicon atom.

According to one advantageous embodiment, compound X is chosen from polyorganosiloxanes comprising siloxane units of formula:

$${R}_{m} {R}^{′} {SiO}_{\frac{( 3 - m )}{2}} \qquad ( I )$$

in which:
R represents a linear or cyclic monovalent hydro-carbon-based group containing from 1 to 30 carbon atoms, preferably from 1 to 20 and better still from 1 to 10 carbon atoms, for instance a short-chain alkyl radical containing, for example, from 1 to 10 carbon atoms, in particular a methyl radical, or alternatively a phenyl group, preferably a methyl radical,
m is equal to 1 or 2, and
R′ represents:
   an unsaturated aliphatic hydrocarbon-based group containing from 2 to 10 and preferably from 2 to 5 carbon atoms, for instance a vinyl group or a group —R″-CH═CHR′″ in which R″ is a divalent aliphatic hydrocarbon-based chain containing from 1 to 8 carbon atoms, bonded to the silicon atom and R′″ is a hydrogen atom or an alkyl radical containing from 1 to 4 carbon atoms, preferably a hydrogen atom; groups R′ that may be mentioned include vinyl and allylic groups and mixtures thereof; or
   an unsaturated cyclic hydrocarbon-based group containing from 5 to 8 carbon atoms, for instance a cyclohexenyl group.

Preferably, R′ is an unsaturated aliphatic hydrocarbon-based group, preferably a vinyl group.

According to one embodiment, R represents an alkyl radical having from 1 to 10 carbon atoms or alternatively a phenyl group, and preferably a methyl radical, and R′ is a vinyl group.

According to one particular embodiment, the polyorgano-siloxane also comprises units of formula:

$${R}_{n} {SiO}_{\frac{( 4 - n )}{2}} \qquad ( II )$$

in which R is a group as defined above, and n is equal to 1, 2 or 3.

According to one variant, compound X may be a silicone resin comprising at least two ethylenic unsaturations, the resin being capable of reacting with compound Y via hydrosilylation in the presence of a catalyst. Examples that may be mentioned include resins of MQ or MT type themselves bearing —CH═CH₂ unsaturated reactive ends.

These resins are crosslinked organosiloxane polymers.

The nomenclature of silicone resins is known under the name “MDTQ”, the resin being described as a function of the various siloxane monomer units it comprises, each of the letters M, D, T and Q characterizing a type of unit.

The letter M represents the monofunctional unit of formula (CH₃)₃SiO_{1/2}, the silicon atom being bonded to only one oxygen atom in the polymer comprising this unit.

The letter D means a difunctional unit (CH₃)₂SiO_{2/2} in which the silicon atom is bonded to two oxygen atoms. The letter T represents a trifunctional unit of formula (CH₃) SiO_{3/2}

In the units M, D and T defined above, at least one of the methyl groups may be substituted with a group R other than a methyl group, such as a hydrocarbon-based radical (especially alkyl) containing from 2 to 10 carbon atoms or a phenyl group, or alternatively a hydroxyl group.

Finally, the letter Q means a tetrafunctional unit SiO_{4/2} in which the silicon atom is bonded to four hydrogen atoms, which are themselves bonded to the rest of the polymer. Examples of such resins that may be mentioned include MT silicone resins such as poly(phenylvinylsilsesquioxane), for instance the product sold under the reference SST-3PV1 by the company Gelest.

Preferably, compounds X comprise from 0.01% to 1% by weight of unsaturated aliphatic groups.

Advantageously, compound X is chosen from polyorgano-polysiloxanes, especially those comprising the siloxane units (I) and optionally (II) described above.

Compound Y then comprises at least two free Si—H groups (hydrogenosilane groups).

Compound Y may be chosen advantageously from polyorganosiloxanes comprising at least one alkylhydrogenosiloxane unit having the following formula:

$${R}_{p} {HSiO}_{\frac{( 3 - p )}{2}} \qquad ( III )$$

in which:

R represents a linear or cyclic monovalent hydrocarbon-based group containing from 1 to 30 carbon atoms, for instance an alkyl radical containing from 1 to 30 carbon atoms, preferably from 1 to 20 and better still from 1 to 10 carbon atoms, in particular a methyl radical, or alternatively a phenyl group, and p is equal to 1 or 2. Preferably, R is a hydrocarbon-based group, preferably methyl.

These polyorganosiloxane compounds Y containing alkyl-hydrogenosiloxane units may also comprise units of formula:

$${R}_{n} {SiO}_{\frac{( 4 - n )}{2}} \qquad ( II )$$

as defined above.

Compound Y may be a silicone resin comprising at least one unit chosen from the units M, D, T and Q as defined above and comprising at least one Si—H group, such as the poly(methylhydridosilsesquioxanes) sold under the reference SST-3 MH1.1 by the company Gelest.

Preferably, these polyorganosiloxane compounds Y comprise from 0.5% to 2.5% by weight of Si—H groups.

Advantageously, the radicals R represent a methyl group in formulae (I), (II) and (III) above.

Preferably, these polyorganosiloxanes Y comprise end groups of formula (CH₃)₃SiO_{1/2}.

Advantageously, the polyorganosiloxanes Y comprise at least two alkylhydrogenosiloxane units of formula —(H₃C)(H) SiO— and optionally comprise —(H₃C)₂SiO— units. Such polyorganosiloxane compounds Y containing hydrogenosilane groups are described, for example, in document EP 0 465 744.

According to one variant, compound X is chosen from organic oligomers or polymers (the term “organic” means compounds whose main chain is not silicone-based, preferably compounds comprising no silicon atoms) or from organic/silicone hybrid polymers or oligomers, the oligomers or polymers bearing at least 2 reactive unsaturated aliphatic groups, compound Y being chosen from the polyorganosiloxanes Y containing hydrogeno-silane groups mentioned above.

According to one embodiment, the organic or hybrid organic/silicone compounds X bearing at least two respective unsaturated aliphatic groups bear at least one polar group as described above.

Compound X, of organic nature, may then be chosen from vinyl or (meth)acrylic polymers or oligomers, polyesters, polyurethanes and/or polyureas, polyethers, perfluoropolyethers, polyolefins such as polybutene or polyisobutylene, dendrimers and organic hyperbranched polymers, or mixtures thereof.

In particular, the organic polymer or the organic part of the hybrid polymer may be chosen from the following polymers:
a) ethylenically unsaturated polyesters:

This is a group of polymers of polyester type containing at least two ethylenic double bonds, randomly distributed in the main polymer chain. These unsaturated polyesters are obtained by polycondensation of a mixture:
of linear or branched aliphatic or cycloaliphatic dicarboxylic acids especially containing from 3 to 50 carbon atoms, preferably from 3 to 20 and better from 3 to 10 carbon atoms, such as adipic acid or sebacic acid, of aromatic dicarboxylic acids especially containing from 8 to 50 carbon atoms, preferably from 8 to 20 and better still from 8 to 14 carbon atoms, such as phthalic acids, especially terephthalic acid, and/or of dicarboxylic acids derived from ethylenically unsaturated fatty acid dimers such as the oleic or linoleic acid dimers described in patent application EP-A-959 066 (paragraph [0021]) sold under the names Pripol® by the company Uniqema or Empol® by the company Henkel, all these diacids needing to be free of polymerizable ethylenic double bonds,
of linear or branched aliphatic or cycloaliphatic diols especially containing from 2 to 50 carbon atoms, preferably from 2 to 20 and better from 2 to 10 carbon atoms, such as ethylene glycol, diethylene glycol, propylene glycol, 1,4-butanediol or cyclohexanedimethanol, of aromatic diols containing from 6 to 50 carbon atoms, preferably from 6 to 20 and better from 6 to 15 carbon atoms, such as bisphenol A and bisphenol B, and/or of diol dimers obtained from the reduction of fatty acid dimers as defined above, and
of one or more dicarboxylic acids or anhydrides thereof comprising at least one polymerizable ethylenic double bond and containing from 3 to 50 carbon atoms, preferably from 3 to 20 and better still from 3 to 10 carbon atoms, such as maleic acid, fumaric acid or itaconic acid.
b) polyesters containing (meth)acrylate side groups and/or end groups:

This is a group of polymers of polyester type obtained by polycondensation of a mixture:
of linear or branched aliphatic or cycloaliphatic dicarboxylic acids especially containing from 3 to 50 carbon atoms, preferably from 3 to 20 and better still from 3 to 10 carbon atoms, such as adipic acid or sebacic acid, of aromatic dicarboxylic acids especially containing from 8 to 50 carbon atoms, preferably from 8 to 20 and better still from 8 to 14 carbon atoms, such as phthalic acids, especially terephthalic acid, and/or of dicarboxylic acids derived from ethylenically unsaturated fatty acid dimers such as the oleic acid or linoleic acid dimers described in patent application EP-A-959 066 (paragraph [0021]) sold under the names Pripol® by the company Uniqema or Empol® by the company Henkel, all these diacids needing to be free of polymerizable ethylenic double bonds,
of linear or branched aliphatic or cycloaliphatic diols especially containing from 2 to 50 carbon atoms, preferably from 2 to 20 and better still from 2 to 10 carbon atoms, such as ethylene glycol, diethylene glycol, propylene glycol, 1,4-butanediol or cyclohexanedimethanol, of aromatic diols containing from 6 to 50 carbon atoms, preferably from 6 to 20 and better still from 6 to 15 carbon atoms, such as bisphenol A and bisphenol B, and
of at least one ester of (meth)acrylic acid and of a diol or polyol containing from 2 to 20 carbon atoms and preferably from 2 to 6 carbon atoms, such as 2-hydroxyethyl(meth)acrylate, 2-hydroxypropyl(meth)acrylate or glycerol methacrylate.

These polyesters differ from those described above in point a) by the fact that the ethylenic double bonds are not located in the main chain but on side groups or at the end of the chains. These ethylenic double bonds are those of the (meth)acrylate groups present in the polymer.

Such polyesters are sold, for example, by the company UCB under the names Ebecryl® (Ebecryl® 450: molar mass 1600, on average 6 acrylate functions per molecule, Ebecryl® 652: molar mass 1500, on average 6 acrylate functions per molecule, Ebecryl® 800: molar mass 780, on average 4 acrylate functions per molecule, Ebecryl® 810: molar mass 1000, on average 4 acrylate functions per molecule, Ebecryl® 50 000: molar mass 1500, on average 6 acrylate functions per molecule)
c) polyurethanes and/or polyureas containing (meth)-acrylate groups, obtained by polycondensation
   of aliphatic, cycloaliphatic and/or aromatic diisocyanates, triisocyanates and/or polyiso-cyanates especially containing from 4 to 50 and preferably from 4 to 30 carbon atoms, such as hexamethylene diisocyanate, isophorone diiso-cyanate, toluene diisocyanate, diphenylmethane diisocyanate or isocyanurates of formula

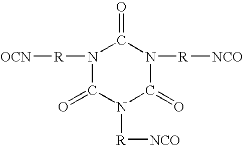

resulting from the trimerization of 3 molecules of diisocyanates OCN—R—CNO, in which R is a linear, branched or cyclic hydrocarbon-based radical comprising from 2 to 30 carbon atoms;
of polyols, especially of diols, free of polymerizable ethylenic unsaturations, such as 1,4-butanediol, ethylene glycol or trimethylol-propane, and/or of aliphatic, cycloaliphatic and/or aromatic polyamines, especially diamines, especially containing from 3 to 50 carbon atoms, such as ethylenediamine or hexamethylenediamine, and
of at least one ester of (meth)acrylic acid and of a diol or polyol containing from 2 to 20 carbon atoms and preferably from 2 to 6 carbon atoms, such as 2-hydroxyethyl(meth)acrylate, 2-hydroxypropyl(meth)acrylate or glycerol methacrylate.

Such polyurethanes/polyureas containing acrylate groups are sold, for example, under the name SR 368 (tris(2-hydroxyethyl) isocyanurate-triacrylate) or Craynor® 435 by the company Cray Valley, or under the name Ebecryl® by the company UCB (Ebecryl® 210: molecular mass 1500, 2 acrylate functions per molecule, Ebecryl® 230: molecular mass 5000, 2 acrylate functions per molecule, Ebecryl® 270: molecular mass 1500, 2 acrylate functions per molecule, Ebecryl® 8402: molecular mass 1000, 2 acrylate functions per molecule, Ebecryl® 8804: molecular mass 1300, 2 acrylate functions per molecule, Ebecryl® 220: molecular mass 1000, 6 acrylate functions per molecule, Ebecryl® 2220: molecular mass 1200, 6 acrylate functions per molecule, Ebecryl® 1290: molecular mass 1000, 6 acrylate functions per molecule, Ebecryl® 800: molecular mass 800, 6 acrylate functions per molecule).

Mention may also be made of the water-soluble aliphatic diacrylate polyurethanes sold under the names Ebecryl® 2000, Ebecryl® 2001 and Ebecryl® 2002, and the diacrylate polyurethanes in aqueous dispersion sold under the trade names IRR® 390, IRR® 400, IRR® 422 and IRR® 424 by the company UCB.
d) polyethers containing (meth)acrylate groups obtained by esterification, with (meth)acrylic acid, of the hydroxyl end groups of C₁₋₄ alkylene glycol homopolymers or copolymers, such as polyethylene glycol, polypropylene glycol, copolymers of ethylene oxide and of propylene oxide preferably having a weight-average molecular mass of less than 10,000, and polyethoxylated or polypropoxylated trimethylolpropane.

Polyoxyethylene di(meth)acrylates of suitable molar mass are sold, for example, under the names SR 259, SR 344, SR 610, SR 210, SR 603 and SR 252 by the company Cray Valley or under the name Ebecryl® 11 by UCB. Polyethoxylated trimethylolpropane triacrylates are sold, for example, under the names SR 454, SR 498, SR 502, SR 9035 and SR 415 by the company Cray Valley or under the name Ebecryl® 160 by the company UCB. Polypropoxylated trimethylolpropane triacrylates are sold, for example, under the names SR 492 and SR 501 by the company Cray Valley.
e) epoxyacrylates obtained by reaction between
   at least one diepoxide chosen, for example, from:
      (i) bisphenol A diglycidyl ether,
      (ii) a diepoxy resin resulting from the reaction between bisphenol A diglycidyl ether and epichlorohydrin,
      (iii) an epoxy ester resin containing α,ω-diepoxy end groups resulting from the condensation of a dicarboxylic acid containing from 3 to 50 carbon atoms with a stoichiometric excess of (i) and/or (ii), and
      (iv) an epoxy ether resin containing α,ω-diepoxy end groups resulting from the condensation of a diol containing from 3 to 50 carbon atoms with a stoichiometric excess of (i) and/or (ii),
      (v) natural or synthetic oils bearing at least 2 epoxide groups, such as epoxidized soybean oil, epoxidized linseed oil or epoxidized vernonia oil,
      (vi) a phenol-formaldehyde polycondensate (Novolac® resin), the end groups and/or side groups of which have been epoxidized,
         and
   one or more carboxylic acids or polycarboxylic acids comprising at least one ethylenic double bond in the α,β-position relative to the carboxylic group, for instance (meth)acrylic acid or crotonic acid or esters of (meth)acrylic acid and of a diol or polyol containing from 2 to 20 carbon atoms and preferably from 2 to 6 carbon atoms, such as 2-hydroxyethyl(meth)acrylate.

Such polymers are sold, for example, under the names SR 349, SR 601, CD 541, SR 602, SR 9036, SR 348, CD 540, SR 480 and CD 9038 by the company Cray Valley, under the names Ebecryl® 600, Ebecryl® 609, Ebecryl® 150, Ebecryl® 860 and Ebecryl® 3702 by the company UCB and under the names Photomer® 3005 and Photomer® 3082 by the company Henkel.
f) poly(C₁₋₅₀ alkyl(meth)acrylates), the alkyl being linear, branched or cyclic, comprising at least two functions containing an ethylenic double bond borne by the hydrocarbon-based side chains and/or end chains.

Such copolymers are sold, for example, under the names IRR® 375, OTA® 480 and Ebecryl® 2047 by the company UCB.
g) polyolefins such as polybutene or polyisobutylene,
h) perfluoropolyethers containing acrylate groups obtained by esterification, for example with (meth)acrylic acid, of perfluoropolyethers bearing hydroxyl side groups and/or end groups.

Such α,ω-diol perfluoropolyethers are described especially in EP-A-1 057 849 and are sold by the company Ausimont under the name Fomblin® Z Diol.
i) hyperbranched dendrimers and polymers bearing (meth)acrylate or (meth)acrylamide end groups obtained, respectively, by esterification or amidation of hyperbranched dendrimers and polymers containing hydroxyl or amino end functions, with (meth)acrylic acid.

Dendrimers (from the Greek dendron tree) are “arborescent”, i.e. highly branched, polymer molecules invented by D. A. Tomalia and his team at the start of the 1990s (Donald A. Tomalia et al., Angewandte Chemie, Int. Engl. Ed., Vol. 29, No. 2, pages 138-175). These are structures constructed about a central unit that is generally polyvalent. About this central unit are linked, in a fully determined structure, branched chain-extending units, thus giving rise to monodispersed symmetrical macromolecules having a well-defined chemical and stereochemical structure. Dendrimers of polyamidoamine type are sold, for example, under the name Starburst® by the company Dendritech.

Hyperbranched polymers are polycondensates, generally of polyester, polyamide or polyethyleneamine type, obtained from multifunctional monomers, which have an arborescent structure similar to that of dendrimers but are much less regular than dendrimers (see, for example, WO-A-93/17060 and WO 96/12754).

The company Perstorp sells hyperbranched polyesters under the name Boltorn®. Hyperbranched polyethylene-amines will be found under the name Comburst® from the company Dendritech. Hyperbranched poly(esteramides) containing hydroxyl end groups are sold by the company DSM under the name Hybrane®.

These hyperbranched dendrimers and polymers esterified or amidated with acrylic acid and/or methacrylic acid are distinguished from the polymers described in points a) to h) above by the very large number of ethylenic double bonds present. This high functionality, usually greater than 5, makes them particularly useful by allowing them to act as “crosslinking nodes”, i.e. sites of multiple crosslinking.

These dendritic and hyperbranched polymers may thus be used in combination with one or more of the polymers and/or oligomers a) to h) above.

1a—Additional reactive compounds

According to one embodiment, the compositions comprising compound X and/or Y may also comprise an additional reactive compound such as:
organic or mineral particles comprising at their surface at least 2 unsaturated aliphatic groups: mention may be made, for example, of silicas surface-treated, for example, with silicone compounds containing vinyl groups, for instance cyclotetramethyltetravinylsiloxane-treated silica,
silazane compounds such as hexamethyldisilazane.
   1b—Catalyst

The hydrosilylation reaction is performed in the presence of a catalyst that may be present with one or the other of the compounds X or Y or may be present in isolation. For example, this catalyst may be present in the composition in an encapsulated form if the two compounds X and Y, whose interaction it must bring about, are present in this same composition in an unencapsulated form or, conversely, it may be present therein in an unencapsulated form if at least one of the compounds X and Y is present in the composition in an encapsulated form. The catalyst is preferably platinum-based or tin-based.

Examples that may be mentioned include platinum-based catalysts deposited on a support of silica gel or charcoal powder (coal), platinum chloride, platinum salts and chloroplatinic acids.

Chloroplatinic acids in hexahydrate or anhydrous form, which are readily dispersible in organosilicone media, are preferably used.

Mention may also be made of platinum complexes such as those based on chloroplatinic acid hexahydrate and on divinyltetramethyldisiloxane.

The catalyst may be present in a content ranging from 0.0001% to 20% by weight relative to the total weight of the composition comprising it.

Compounds X and/or Y may be combined with polymerization inhibitors or retarders, and more particularly catalyst inhibitors.

Mention may be made, in a non-limiting manner, of cyclic polymethylvinylsiloxanes, and in particular tetravinyltetramethylcyclotetrasiloxane, and acetylenic alcohols, which are preferably volatile, such as methylisobutynol.

The presence of ionic salts such as sodium acetate may have an influence on the rate of polymerization of the compounds.

By way of example of a combination of compounds X and Y that react via hydrosilylation in the presence of a catalyst, mention may be made of the following references sold by the company Dow Corning: DC7-9800 Soft Skin Adhesive Parts A & B,

and also the combination of mixtures A and B below sold by the company Dow Corning:

MIXTURE A:

| | | Content | |
|---|---|---|---|
| Ingredient (INCI name) | CAS No. | (%) | Function |
| Dimethylsiloxane, | 68083-19-2 | 55-95 | Polymer |
| Dimethylvinylsiloxy-terminated | | | |
| Silica Silylate | 68909-20-6 | 10-40 | Filler |
| 1,3-Diethenyl-1,1,3,3-Tetra- | 68478-92-2 | Trace | Catalyst |
| methyldisiloxane complexes | | | |
| Tetramethyldivinyldisiloxane | 2627-95-4 | 0.1-1 | Polymer |

MIXTURE B:

| | | Content | |
|---|---|---|---|
| Ingredient (INCI name) | CAS No. | (%) | Function |
| Dimethylsiloxane, | 68083-19-2 | 55-95 | Polymer |
| Dimethylvinylsiloxy-terminated | | | |
| Silica Silylate | 68909-20-6 | 10-40 | Filler |
| Dimethyl, | 68037-59-2 | 1-10 | Polymer |
| Methylhydrogenosiloxane, | | | |
| trimethylsiloxy-terminated | | | |

Advantageously, compounds X and Y are chosen from silicone compounds capable of reacting via hydrosilylation in the presence of a catalyst; in particular, compound X is chosen from polyorganosiloxanes comprising units of formula (I) described above and compound Y is chosen from organosiloxanes comprising alkylhydrogenosiloxane units of formula (III) described above.

According to one particular embodiment, compound X is a polydimethylsiloxane containing vinyl end groups and compound Y is a polymethylhydrogenosiloxane.

2—Compounds X and Y capable of reacting via condensation

According to one embodiment, the invention relates to a cosmetic kit that can be used for the care and/or make-up of keratinous substance(s), notably of the skin, comprising at least two compositions that are different and are packaged separately with at least one of the compositions being of the emulsion type and comprising at least one cosmetic and/or dermatological active, the kit comprising one or more compounds X, one or more compounds Y, and optionally at least one catalyst, with at least one of compounds X and Y being a silicone compound and said compounds X and Y being capable of reacting together by a condensation reaction when they are brought into contact with one another, and in which the compounds X, Y and the catalyst, when it is present, are not present simultaneously in the same composition.

In particular, the cosmetic active is a moisturizer agent which represents at least 0.1% by weight, preferably at least 0.1% by weight relative to the total weight of the composition.

According to this embodiment, compounds X and Y are capable of reacting via condensation, either in the presence of water (hydrolysis) by reaction of 2 compounds bearing alkoxysilane groups, or via “direct” condensation by reaction of a compound bearing alkoxy-silane group(s) and a compound bearing silanol group(s) or by reaction of 2 compounds bearing silanol group(s).

When the condensation is performed in the presence of water, this water may in particular be ambient moisture, residual water of the skin, the lips, the eyelashes and/or the nails, or the water provided by an external source, for example premoistening of the keratin fibres (for example with a mister, or natural or artificial tears).

In this mode of reaction via condensation, compounds X and Y, which may be identical or different, may thus be chosen from silicone compounds whose main chain comprises at least two alkoxysilane groups and/or at least two silanol (Si—OH) groups, on the side and/or at the end of the chain.

According to one embodiment, compound X and/or compound Y bears at least one polar group, as described above, capable of forming at least one hydrogen bond with keratin materials.

In one advantageous embodiment, compounds X and/or Y are chosen from polyorganosiloxanes comprising at least two alkoxysilane groups. The term “alkoxysilane group” means a group comprising at least one —Si—OR portion, R being an alkyl group containing from 1 to 6 carbon atoms.

Compounds X and Y are especially chosen from poly-organosiloxanes comprising alkoxysilane end groups, more specifically those comprising at least 2 alkoxysilane end groups and preferably trialkoxysilane end groups.

These compounds X and/or Y preferably predominantly comprise units of formula:

R⁹ₛSiO_{(4-s)/2} (IV)

in which R⁹ independently represents a radical chosen from alkyl groups containing from 1 to 6 carbon atoms, phenyl and fluoroalkyl groups, and s is equal to 0, 1, 2 or 3. Preferably, R⁹ independently represents an alkyl group containing from 1 to 6 carbon atoms. Alkyl groups that may especially be mentioned include methyl, propyl, butyl, and hexyl, and mixtures thereof, preferably methyl or ethyl. A fluoroalkyl group that may be mentioned is 3,3,3-trifluoropropyl.

According to one particular embodiment, compounds X and Y, which may be identical or different, are polyorgano-siloxanes comprising units of formula:

(R⁹₂SiO²)_{f}— (V)

in which R⁹ is as described above, preferably R⁹ is a methyl radical, and f is such that the polymer advantageously has a viscosity at 25° C. ranging from 0.5 to 3000 Pa·s and preferably ranging from 5 to 150 Pa·s. f is especially a number ranging from 2 to 50 000, particularly from 3 to 3000 and more particularly from 5 to 1000.

These polyorganosiloxane compounds X and Y comprise at least 2 trialkoxysilane end groups per polymer molecule, the groups having the following formula:

-ZSiR¹ₓ(OR)₃₋ₓ, (VI)

in which:
the radicals R independently represent a methyl, ethyl, n-propyl, isopropyl, n-butyl, sec-butyl or isobutyl group, preferably a methyl or ethyl group,
R¹ is a methyl or ethyl group,
x is equal to 0 or 1 and preferably x is equal to 0, and
Z is chosen from: divalent hydrocarbon-based groups not comprising any ethylenic unsaturation and containing from 1 to 18 carbon atoms, preferably from 2 to 18 carbon atoms (alkylene groups), combinations of divalent hydrocarbon-based radicals and of siloxane segments of formula (IX) below:

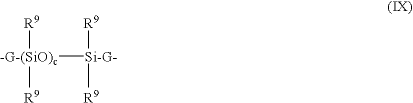

R⁹ being as described above, G is a divalent hydrocarbon-based radical not comprising any ethylenic unsaturation and containing from 1 to 18 carbon atoms, preferably from 2 to 18 carbon atoms and c is an integer ranging from 1 to 6.

Z and G may be chosen especially from alkylene groups such as methylene, ethylene, propylene, butylene, pentylene and hexylene, and arylene groups such as phenylene.

Preferably, Z is an alkylene group, and better still ethylene.

These polymers may contain on average at least 1.2 tri-alkoxysilane end groups or end chains per molecule, and preferably on average at least 1.5 trialkoxysilane end groups per molecule. Since these polymers may contain at least 1.2 trialkoxysilane end groups per molecule, some may comprise other types of end groups such as end groups of formula CH₂═CH—SiR⁹₂— or of formula R⁶₃—Si—, in which R⁹ is as defined above and each group R⁶ is independently chosen from groups R⁹ and vinyl. Examples of such end groups that may be mentioned include trimethoxysilane, triethoxysilane, vinyldimethoxysilane and vinylmethyloxyphenylsilane groups.

Such polymers are especially described in documents U.S. Pat. No. 3,175,993, U.S. Pat. No. 4,772,675, U.S. Pat. No. 4,871,827, U.S. Pat. No. 4,888,380, U.S. Pat. No. 4,898,910, U.S. Pat. No. 4,906,719 and U.S. Pat. No. 4,962,174, the content of which is incorporated into the present patent application by reference.

As compound X and/or Y, mention may be made in particular polyorganosiloxanes selected from the polymers of formula

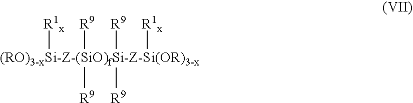

in which R, R¹, R⁹, Z, x and f are as described above.

Compounds X and/or Y may also comprise a mixture of polymer of formula (VII) above with polymers of formula (VIII) below:

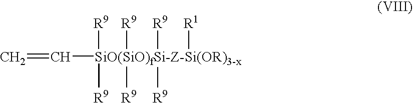

in which R, R¹, R⁹, Z, x and f are as described above.

When the polyorganosiloxane compound X and/or Y containing alkoxysilane group(s) comprises such a mixture, the various polyorganosiloxanes are present in contents such that the organosilyl end chains represent less than 40% and preferably less than 25% by number of the end chains.

The polyorganosiloxane compounds X and/or Y that are particularly preferred are those of formula (VII) described above. Such compounds X and/or Y are described, for example, in document WO 01/96450.

As indicated above, compounds X and Y may be identical or different.

In particular, compounds X and Y can represent a mixture of polydimethylsiloxanes with methoxysilane groups.

According to one variant, one of the two reactive compounds X or Y is of silicone nature and the other is of organic nature. For example, compound X is chosen from organic oligomers or polymers or organic/silicone hybrid oligomers or polymers, the polymers or oligomers comprising at least two alkoxysilane groups, and Y is chosen from silicone compounds such as the polyorganosiloxanes described above. In particular, the organic oligomers or polymers are chosen from vinyl, (meth)acrylic, polyester, polyamide, polyurethane and/or polyurea, polyether, polyolefin or perfluoro-polyether oligomers or polymers, and hyperbranched organic dendrimers and polymers, and mixtures thereof.

According to one embodiment, compound X of organic nature or of hybrid organic/silicone nature bears at least one polar group, as described above, capable of forming at least one hydrogen bond with the keratin material.

The organic polymers of vinyl or (meth)acrylic nature bearing alkoxysilane side groups may in particular be obtained via copolymerization of at least one organic vinyl or (meth)acrylic monomer with a (meth)acryloxy-propyltrimethoxysilane, a vinyltrimethoxysilane, a vinyltriethoxysilane, an allyltrimethoxysilane, etc.

Examples that may be mentioned include the (meth)acrylic polymers described in the document by Kusabe, M., Pitture e Verniei—European Coating; 12-B, pages 43-49, 2005, and especially the polyacrylates containing alkoxysilane groups referenced as MAX from Kaneka or those described in the publication by Probster, M., Adhesion-Kleben & Dichten, 2004, 481 (1-2), pages 12-14.

The organic polymers resulting from a polycondensation or a polyaddition, such as polyesters, polyamides, polyurethanes and/or polyureas, and polyethers, and bearing alkoxysilane side and/or end groups, may result, for example, from the reaction of an oligomeric prepolymer as described above with one of the following silane coreagents bearing at least one alkoxysilane group: aminopropyltrimethoxysilane, aminopropyltri-ethoxysilane, aminoethylaminopropyltrimethoxysilane, glycidoxypropyltrimethoxysilane, glycidoxypropyltri-ethoxysilane, epoxycyclohexylethyltrimethoxysilane, mercaptopropyltrimethoxysilane.

Examples of polyethers and polyisobutylenes containing alkoxysilane groups are described in the publication by Kusabe, M., Pitture e Verniei—European Coating; 12-B, pages 43-49, 2005. As examples of polyurethanes containing alkoxysilane end groups, mention may be made of those described in the document by Probster, M., Adhesion-Kleben & Dichten, 2004, 481 (1-2) pages 12-14 or those described in the document by Landon, S., Pitture e Verniei vol. 73, No. 11, pages 18-24, 1997 or in the document by Huang, Mowo, Pitture e Verniei vol. 5, 2000, pages 61-67; mention may be made especially of the polyurethanes containing alkoxysilane groups from OSI-WITCO-GE.

Polyorganosiloxane compounds X and/or Y that may be mentioned include resins of MQ or MT type themselves bearing alkoxysilane and/or silanol ends, for instance the poly(isobutylsilsesquioxane) resins functionalized with silanol groups sold under the reference SST-S7C41 (3 Si—OH groups) by the company Gelest.

2a—Additional Reactive Compound

According to one embodiment, compound X and/or Y may also be combined with an additional reactive compound comprising at least two alkoxysilane or silanol groups. Mention may be made, for example, of one or more organic or mineral particles comprising at their surface alkoxysilane and/or silanol groups, for instance fillers surface-treated with such groups.

2b—Catalyst

The condensation reaction may be performed in the presence of a metal-based catalyst that may be present in one or the other of the compounds X or Y or may be present in isolation. For example, this catalyst may be present in the composition in an encapsulated form if the two compounds X and Y, whose interaction it must bring about, are present in this same composition in an unencapsulated form order, conversely, it may be present in an unencapsulated form if at least one of the compounds X and Y is present in the composition in an encapsulated form. The catalyst that is useful in this type of reaction is preferably a titanium-based catalyst.

Mention may be made especially of the tetraalkoxy-titanium-based catalysts of formula

Ti(OR²)_{y}(OR³)_{4-y},

in which R² is chosen from tertiary alkyl radicals such as tert-butyl, tert-amyl and 2,4-dimethyl-3-pentyl; R³ represents an alkyl radical containing from 1 to 6 carbon atoms, preferably a methyl, ethyl, n-propyl, isopropyl, n-butyl, sec-butyl or hexyl group and y is a number ranging from 3 to 4 and better still from 3.4 to 4.

The catalyst may be present in a content ranging from 0.0001% to 20% by weight relative to the total weight of the composition containing it.

2c—Diluent

The useful compositions comprising X and/or Y may also comprise a volatile silicone oil (or diluent) for reducing the viscosity of the composition. This oil may be chosen from short-chain linear silicones such as hexamethyldisiloxane or octamethyltrisiloxane, and cyclic silicones such as octamethylcyclotetrasiloxane and decamethylcyclopentasiloxane, and mixtures thereof.

This silicone oil may represent from 5% to 95% and preferably from 10% to 80% by weight relative to the weight of each composition.

As examples of a combination of compounds X and Y bearing alkoxysilane groups and reacting via condensation, mention may be made of the mixtures A′ and B′ below sold by the company Dow Corning.

Mixture A′:

| | | Content | |
|---|---|---|---|
| Ingredient (INCI name) | CAS No. | (%) | Function |
| Bis-Trimethoxysiloxy- | PNM8711-76 | 25-45 | Polymer |
| ethyl | | | |
| Tetramethyldisiloxyethyl | | | |
| Dimethicone (1) | | | |
| Silica Silylate | 68909-20-6 | 5-20 | Filler |
| Disiloxane | 107-46-0 | 30-70 | Solvent |

It should moreover be noted that the identical compounds X and Y are combined in the mixture A′ (cf. (1)).

Mixture B′:

| Ingredient (INCI name) | CAS No. | Content (%) | Function |
|---|---|---|---|
| Disiloxane | 107-46-0 | 80-99 | Solvent |
| Tetra T Butyl Titanate | — | 1-20 | Catalyst |

### 3—Crosslinking in the Presence of Peroxide;

According to one embodiment, the invention relates to a cosmetic kit that can be used for the care and/or make-up of keratinous substance(s), notably of the skin, comprising at least two compositions that are different and are packaged separately with at least one of the compositions being of the emulsion type and comprising at least one cosmetic and/or dermatological active, the kit comprising one or more compounds X, one or more compounds Y, and at least one peroxide, with at least one of compounds X and Y being a silicone compound and said compounds X and Y being capable of reacting together by a crosslinking reaction in the presence of a peroxide when they are brought into contact with one another, and in which the compounds X, Y and the peroxide are not present simultaneously in the same composition.

In particular, the cosmetic active is a moisturizer agent which represents at least 0.1% by weight, preferably at least 0.1% by weight relative to the total weight of the composition.

This reaction is preferably performed by heating to a temperature of greater than or equal to 50° C., preferably greater than or equal to 80° C., which may be up to 120° C.

The identical or different compounds X and Y comprise in this case at least two —CH₃ side groups and/or at least two side chains bearing a —CH₃ group.

Compounds X and Y are preferably silicone compounds and may be chosen, for example, from high molecular weight non-volatile linear polydimethylsiloxanes, with a degree of polymerization of greater than 6, containing at least two —CH₃ side groups bonded to the silicon atom and/or at least two side chains bearing a —CH₃ group. Mention may be made, for example, of polymers described in the “Reactive Silicones” catalogue from the company Gelest Inc., Edition 2004, page 6, and especially vinylmethylsiloxane-dimethylsiloxane copolymers (also referred to as gums) with molecular weights ranging from 500 000 to 900 000 and a viscosity of greater than 2 000 000 cSt.

As peroxides that may be used in the context of the invention, mention may be made of benzoyl peroxide and 2,4-dichlorobenzoyl peroxide, and mixtures thereof.

According to one embodiment, the hydrosilylation reaction in the presence of a catalyst, or the condensation reaction, or alternatively the crosslinking reaction in the presence of a peroxide, between compounds X and Y is accelerated by supplying heat, for example by raising the temperature of the system to between 25° C. and 180° C.

In general, irrespective of the type of reaction via which compounds X and Y react together, the mole percentage of X relative to all of the compounds X and Y. i.e. the ratio X/(X+Y)×100, may range from 5% to 95%, preferably from 10% to 90% and better still from 20% to 80%.

Similarly, the mole percentage of Y relative to all of the compounds X and Y, i.e. the ratio Y/(X+Y)×100, may range from 5% to 95%, preferably from 10% to 90% and better still from 20% to 80%.

Compound X may have a weight-average molecular mass (Mw) ranging from 150 to 1 000 000, preferably from 200 to 800 000 and more preferably from 200 to 250 000.

Compound Y may have a weight-average molecular mass (Mw) ranging from 200 to 1 000 000, preferably from 300 to 800 000 and more preferably from 500 to 250 000.

Compound X may represent from 0.1% to 95% by weight, preferably from 1% to 90% and better still from 5% to 80% by weight relative to the total weight of the composition containing it.

Compound Y may represent from 0.1% to 95% by weight, preferably from 1% to 90% and better still from 5% to 80% by weight relative to for example by way to the total weight of the composition containing it.

The ratio between the compounds X and Y may be varied so as to modify the rate of reaction and thus the rate of formation of the film, or alternatively so as to adapt the properties of the film formed (for example its adhesive properties) according to the desired application.

In particular, compounds X and Y may be present in a mole ratio X/Y ranging from 0.05 to 20 and better still from 0.1 to 10.

Compounds X and Y may advantageously be combined with at least one filler. Thus, the kit may comprise in a composition at least one filler chosen from silica and surface-treated silica.

As stated previously, according to one embodiment of the invention, compounds X and Y may be used in the form of a single composition which then contains at least one of them or, where appropriate, the catalyst or the peroxide if necessary for their interaction, in an encapsulated form.

In the context of the present invention, encapsulated forms of core/shell type also referred to as microcapsules or nanocapsules, the shell of which is of polymeric nature and the core contains compound X, compound Y, at least one of these compounds X and Y being encapsulated, where appropriate, with the catalyst or the peroxide if necessary for the interaction of the two compounds, are more particularly considered.

Many techniques are currently available for preparing microcapsules or nanocapsules of this type.

However, according to one preferred mode, the encapsulated forms under consideration according to the invention are nanocapsules and are obtained via a “solvent tipping” technique illustrated especially in documents EP 274 961 and EP 1 552 820.

More particularly, the shell of the nanocapsules of compound X or Y, used according to the invention, is of non-crosslinked, water-insoluble polymeric nature and is insoluble in the core of the capsules.

In general, any polymer of natural or synthetic origin, which is soluble in a water-immiscible solvent, and especially those with a melting point lower than the boiling point of water at atmospheric pressure (100° C.), may be suitable for use. The these polymers may be biodegradable, for instance polyesters, or non-biodegradable.

As illustrations of polymers that are suitable for the invention, mention may be made especially of:
C₂-C₁₂ alkyl cyanoacrylate polymers,
   polymers formed from poly-L-lactides, poly-DL-lactides and polyglycolides, and the corresponding copolymers,
polycaprolactones,
3-hydroxybutyric acid polymers,
copolymers of vinyl chloride and vinyl acetate,
copolymers of methacrylic acid and ester, especially of methacrylic acid and of methacrylic acid ester,
polyvinyl acetophthalate,
cellulose acetophthalate,
polyvinylpyrrolidone-vinyl acetate copolymer,
polyethylene vinyl acetates,
polyacrylonitriles,
polyacrylamides,
polyethylene glycols,
poly(C₁-C₄ hydroxyalkyl methacrylates),
C₁-C₄ carboxylic acid esters of cellulose,
polystyrene and copolymers of styrene and of maleic anhydride, copolymers of styrene and of acrylic acid, styrene-ethylene/butylene-styrene block terpolymers, and styrene-ethylene/propylene-styrene block terpolymers,
styrene alkyl alcohol oligomers,
terpolymers of ethylene, of vinyl acetate and of maleic anhydride,
polyamides,
polyethylenes,
polypropylenes,
organopolysiloxanes including polydimethylsiloxanes,
poly(alkylene adipates),
polyester polyols,
polysilsesquioxane silicone polymers,
dendritic polyesters bearing a hydroxyl end function,
polymers that are water-dispersible but, never-theless, soluble in water-immiscible solvents, for instance: polyesters, poly(esteramides), polyurethanes and vinyl copolymers bearing carboxylic acid and/or sulfonic acid functions and in particular those described in document FR 2 787 729,
block copolymers that are insoluble in water at room temperature and solid at room temperature, containing at least one block of one of the preceding polymers,
mixtures thereof.

These polymers or copolymers may have a weight-average molecular weight of between 1000 and 500 000 and in particular between 1500 and 100 000.

Poly(alkylene adipates), organopolysiloxanes, polycaprolactones, cellulose acetophthalate, cellulose acetobutyrate, cellulose esters and polystyrene, and derivatives thereof, and notably polycaprolactones are most particularly suitable for use in the invention.

Needless to say, a person skilled in the art is capable, by virtue of his knowledge, of adjusting the molecular weight of the selected polymer with respect to its concentration in the solvent in order to have a viscosity of the mixture that is compatible with satisfactory emulsification.

As regards the lipophilic core, it may contain, besides compound X or compound Y, at least one oil. The oil may be chosen from the oils described below for the oily phase. The oil is preferably a silicone oil.

According to one variant of the invention, the encapsulated forms of compound X or compound Y may be coated with a lamellar phase.

As regards the procedure for preparing the nanocapsules that are suitable for use in the invention, a person skilled in the art may refer especially to the teaching of document EP 1 552 820 mentioned below. The choice of surfactants required and the implementation of the process call upon the knowledge of a person skilled in the art.

### Cosmetic and/or Dermatological Active Agents

The cosmetic and/or dermatological active agent(s) present in the kit and/or at least one of the compositions according to the invention may be chosen from moisturizers, desquamating agents, agents for improving the barrier function, depigmenting agents, antioxidants, dermo-decontracting agents, anti-glycation agents, agents for stimulating the synthesis of epidermal and/or epidermal macromolecules and/or for preventing their degradation, agents for stimulating fibroblast or keratinocyte proliferation and/or keratinocyte differentiation, agents for promoting the maturation of the horny envelope, NO-synthase inhibitors, peripheral benzodiazepine receptor (PBR) antagonists, agents for increasing the activity of the sebaceous glands, agents for stimulating the energy metabolism of cells, tensioning agents, lipo-restructuring agents, slimming agents, agents for promoting cutaneous microcirculation, calmatives and/or anti-irritants, sebo-regulating or anti-seborrhoeic agents, astringents, cicatrizing agents, anti-inflammatory agents and anti-acne agents.

A person skilled in the art will choose the active agent(s) as a function of the effect desired on the keratin materials.

In a preferred embodiment, the kit or compositions according to the invention comprise at least one moisturizer agent which represents at least 0.01% by weight, preferably at least 0.1% by weight relative to the total weight of the composition.

The kit or compositions according to the inventions may comprise in addition at least one other cosmetic and/or dermatological active.

For caring for and/or making up aged skin, he will preferably choose at least one active agent chosen from moisturizers, desquamating agents, agents for improving the barrier function, depigmenting agents, antioxidants, dermo-decontracting agents, anti-glycation agents, agents for stimulating the synthesis of dermal and/or epidermal macromolecules and/or for preventing their degradation, agents for stimulating fibroblast or keratinocyte proliferation and/or keratinocyte differentiation, agents for promoting the maturation of the horny envelope, NO-synthase inhibitors, peripheral benzodiazepine receptor (PBR) antagonists, agents for increasing the activity of the sebaceous glands, agents for stimulating the energy metabolism of cells, and agents for promoting the cutaneous microcirculation for the area around the eyes.

The composition may also comprise at least one ingredient such as fillers with a soft-focus effect or agents for promoting the natural coloration of the skin, intended for complementing the biological effects of these active agents or for providing an immediate visual anti-ageing effect.

For caring for and/or making up greasy skin, a person skilled in the art will preferably choose at least one active agent chosen from desquamating agents, sebo-regulating agents or anti-seborrhoeic agents, and astringents.

At least one active agent chosen from anti-acne agents, cicatrizing agents and anti-inflammatory agents will preferably be chosen for caring for and/or making up acne-prone skin.

For slimming care of the body, he will preferably choose an active agent chosen from slimming active agents and active agents for promoting the cutaneous microcirculation.

The composition may also comprise at least one additional ingredient for complementing the biological effect of these active agents or for providing an immediate visual effect; mention may be made especially of matting agents, fillers with a soft-focus effect, fluorescers, agents for promoting the naturally pinkish coloration of the skin, and abrasive fillers or exfoliants.

According to a first mode, the kit or at least one of the compositions according to the invention comprises at least one moisturizer.

According to another mode, the kit or at least one of the compositions according to the invention comprises at least one desquamating agent.

According to another mode, the kit or at least one of the compositions according to the invention comprises at least one agent for improving the barrier function.

According to another mode, the kit or at least one of the compositions according to the invention comprises at least one depigmenting agent.

According to another mode, the kit or at least one of the compositions according to the invention comprises at least one antioxidant.

According to another mode, the kit or at least one of the compositions according to the invention comprises at least one dermo-decontracting agent.

According to another mode, the kit or at least one of the compositions according to the invention comprises at least one anti-glycation agent.

According to another mode, the kit or at least one of the compositions according to the invention comprises at least one agent for stimulating the synthesis of dermal and/or epidermal macromolecules and/or for preventing their degradation.

According to another mode, the kit or at least one of the compositions according to the invention comprises at least one agent for stimulating fibroblast or keratinocyte proliferation and/or keratinocyte differentiation.

According to another mode, the kit or at least one of the compositions according to the invention comprises at least one agent for promoting the maturation of the horny envelope.

According to another mode, the kit or at least one of the compositions according to the invention comprises at least one NO-synthase inhibitor.

According to another mode, the kit or at least one of the compositions according to the invention comprises at least one peripheral benzodiazepine receptor (PBR) antagonist.

According to another mode, the kit or at least one of the compositions according to the invention comprises at least one agent for increasing the activity of the sebaceous glands.

According to another mode, the kit or at least one of the compositions according to the invention comprises at least one agent for stimulating the energy metabolism of cells.

According to another mode, the kit or at least one of the compositions according to the invention comprises at least one tensioning agent.

According to another mode, the kit or at least one of the compositions according to the invention comprises at least one lipo-restructuring agent.

According to another mode, the kit or at least one of the compositions according to the invention comprises at least one slimming agent.

According to another mode, the kit or at least one of the compositions according to the invention comprises at least one agent for stimulating the cutaneous microcirculation.

According to another mode, the kit or at least one of the compositions according to the invention comprises at least one calmative and/or anti-irritant.

According to another mode, the kit or at least one of the compositions according to the invention comprises at least one sebo-regulating or anti-seborrhoeic agent.

According to another mode, the kit or at least one of the compositions according to the invention comprises at least one astringent.

According to another mode, the kit or at least one of the compositions according to the invention comprises at least one cicatrizing agent.

According to another mode, the kit or at least one of the compositions according to the invention comprises at least one anti-inflammatory agent.

According to another mode, the kit or at least one of the compositions according to the invention comprises at least one anti-acne agent.

According to another mode, the kit or at least one of the compositions according to the invention comprises at least one matting agent.

According to another mode, the kit or at least one of the compositions according to the invention comprises at least one filler with a soft-focus effect.

According to another mode, the kit or at least one of the compositions according to the invention comprises at least one fluorescer.

According to another mode, the kit or at least one of the compositions according to the invention comprises at least one agent for promoting the naturally pinkish coloration of the skin.

According to another mode, the kit or at least one of the compositions according to the invention comprises at least abrasive fillers or exfoliants.

Examples of such compounds are described below.

### 1. Moisturizers or Humectants

Moisturizers or humectants that may especially be mentioned include urea and derivatives thereof, especially Hydrovance® sold by National Starch, lactic acid, hyaluronic acid, AHAs, BHAs, sodium pidolate, xylitol, serine, sodium lactate, ectoin and derivatives thereof, collagen, plankton, an extract of Imperata cylindra sold under the name Moist 24® by the company Sederma, acrylic acid homopolymers, for instance Lipidure-HM® from NOF Corporation, beta-glucan from Mibelle-AG-Biochemistry; a mixture of passionflower oil, apricot oil, corn oil and rice bran oil sold by Nestlé under the name NutraLipids®; a C-glycoside derivative such as those described in patent application WO 02/051 828 and in particular C-β-D-xylopyranoside-2-hydroxypropane in the form of a solution containing 30% by weight of active material in a water/propylene glycol mixture (60/40% by weight) such as the product sold by Chimex under the trade name Mexoryl SBB®; an oil of musk rose sold b Nestlé; an oil of the microalga Prophyridium cruentum enriched with zinc, sold by Vincience under the name Algualane Zinc®; spheres of collagen and of chondroitin sulfate of marine of origin (Atelocollagen) sold by the company Engelhard Lyon under the name Marine Filling Spheres; hyaluronic acid spheres such as those sold by the company Engelhard Lyon; arginine; xylitol derivatives, as the mixture of xylitol-polyglucoside/xylitan/xylitol sold under the name AQUAXYL® by the company Seppic; the Phytantriol® or TETRA-METHYL-3,7,11,15 TRI-HYDROXY-1,2,3 HEXA-DECANE sold by the company DSM; the Trehalose or ALPHA-D-GLUCOPYRANOSYL-ALPHA-D-GLUCOPYRANOSIDE; the Fucogel® or BIOSACCHARIDE GUM-1 glycerol derivatives, as the alkylene oxyde glycerol in particular the POLYOXYBUTYLENE POLYOXYETHYLENE POLYOXYPROPYLENE GLYCEROL, or an alkyle ether polyoxyalkyleneglycol/polyethylene glycol copolymere; glycerol esters as the glycerol triacetate; the glucosylglycerides as the 2-O-β-D-Glucopyranosyl-sn-glycérine; siliconed glycerol derivatives as the ones cited in the published application WO06075679 ammonium quaternary salts as the hydroxy propyl bis-hydroxyethyldimonium or Cola Moist 200; dihydroxypropyl tri (C1-C3 alkyl) ammonium derivatives substituted monosaccharides as the chloride hydroxypropyltrimonium glucose or hydroxypropyltrimonium fructose as the ones cited in the published application WO2006045428; the substituted poyols as the chloride hydroxypropyltrimonium sorbitol, as the ones cited in the published application WO2006045364; the esters of stérol and N-acylated amino acids with long chains as the Phytosteryl/octyldodecyl Lauroyl glutamate, or the Cholesteryl/octyldodecyl glutamate for lipophilic medium as cited in the published application WO2007007251, and their mixtures.

The moisturizer that will preferably be used is chosen from urea and derivatives thereof, especially Hydrovance® sold by National Starch, hyaluronic acid, AHAs, BHAs, acrylic acid homopolymers, for instance Lipidure-HM® from NOF Corporation, beta-glucan from Mibelle-AG-Biochemistry; a mixture of passionflower oil, apricot oil, corn oil and rice bran oil sold by Nestléunder the name NutraLipids®; a C-glycoside derivative such as those described in patent application WO 02/051 828 and in particular C-β-D-xylopyranoside-2-hydroxypropane in the form of a solution containing 30′ by weight of active material in a water/propylene glycol mixture (60/40% by weight) such as the product sold by Chimex under the trade name Mexoryl SBB®; an oil of musk rose sold by Nestlé; an oil of the microalga Prophyridium cruentum enriched with zinc, sold by Vincience under the name Algualane Zinc®; spheres of collagen and of chondroitin sulfate of marine of origin (Atelocollagen) sold by the company Engelhard Lyon under the name Marine Filling Spheres hyaluronic acid spheres such as those sold by the company Engelhard Lyon; arginine; xylitol derivatives, as the mixture of xylitol-polyglucoside/xylitan/xylitol sold under the name AQUAXYL® by the company Seppic; the phytantriol or TETRA-METHYL-3,7,11,15 TRI-HYDROXY-1,2,3 HEXA-DECANE sold by the company DSM and their mixtures.

More preferably, the moisturizer is chosen from urea and derivatives thereof, especially Hydrovance® sold by National Starch; a C-glycoside derivative such as those described in patent application WO 02/051 828 and in particular C-β-D-xylopyranoside-2-hydroxypropane in the form of a solution containing 30% by weight of active material in a water/propylene glycol mixture (60/weight) such as the product sold b Chimex under the trade name Mexoryl SBB®; xylitol derivatives as the mixture of xylitol-polyglucoside/xylitan/xylitol sold under the name AQUAXYL® by the company Seppic; the phytantriol or TETRA-METHYL-3,7,11,15 TRI-HYDROXY-1,2,3 HEXA-DECANE sold by the company DSM; and their mixtures.

The lipophilic moisturizer agents might be preferred according to the invention.

In a preferred embodiment, the kit or compositions according to the inventions will contain at least one moisturizer agent chosen from urea and derivatives thereof; hyaluronic acid, AHAs, BHAs, acrylic acid homopolymers, beta-glucan; a mixture of passionflower oil, apricot oil, corn oil and rice bran oil; a C-glycoside derivative and in particular C-β-D-xylopyranoside-2-hydroxypropane in the form of a solution containing 30′ by weight of active material in a water/propylene glycol mixture (60/40% by weight); an oil of musk rose; an oil of the microalga Prophyridium cruentum enriched with zinc; spheres of collagen and of chondroitin sulfate of marine of origin (Atelocollagen); hyaluronic acid spheres; arginine; xylitol derivatives, as the mixture of xylitol-polyglucoside/xylitan/xylitol; the phytantriol or TETRA-METHYL-3,7,11,15 TRI-HYDROXY-1,2,3 HEXA-DECANE sold by the company DSM; and their mixtures.

In a particular embodiment, the moisturizer is chosen from urea and its derivatives.

As preferred urea derivatives, may be cited the hydroxyalkylurea derivatives, where hydroxyalkyl groups are chosen from hydroxyethyl, hydroxypropyl, hydroxybutyl, hydroxypentyl and hydroxyhexyl groups.

There may be mentioned N-(2-hydroxyethyl)urea; N-(2-hydroxypropyl)urea, N-(3-hydroxypropyl)urea; N-(2,3-dihydroxypropyl)urea; N-(2,3,4,5,6-pentahydroxyhexyl)urea; N-methyl-N-(1,3,4,5,6-pentahydroxy-2-hexyl)urea; N-methyl-N′-(1-hydroxy-2-methyl-2-propyl)urea; N-(1-hydroxy-2-methyl-2-propyl)urea; N-(1,3-dihydroxy-2-propyl)urea; N-(trishydroxymethylmethyl)urea; N-ethyl-N′-(2-hydroxyethyl)urea; N,N-bis(2-hydroxyethyl)urea; N,N′-bis(2-hydroxyethyl)urea; N,N-bis(2-hydroxypropyl)urea; N,N′-bis(2-hydroxypropyl)urea; N,N-bis(2-hydroxyethyl)-N′-propylurea; N,N-bis(2-hydroxypropyl)-N′-(2-hydroxy-ethyl)urea; N-tert-butyl-N′-(2-(hydroxyethyl)-N′-(2-(hydroxypropyl)urea; N-(1,3-dihydroxy-2-propyl)-N′-(2-hydroxyethyl)urea; N,N-bis(2-hydroxyethyl)-N′,N′-dimethylurea; N,N,N′,N′-tetrakis(2-hydroxyethyl)urea; N′,N′-bis(2-hydroxyethyl)-N′,N′-bis(2-hydroxypropyl)-urea; and mixtures thereof.

Preferably, the hydroxyalkylurea is N-(2-hydroxy-ethyl)urea, as the one commercially available in the form of a mixture at 50% by weight in water from the company NATIONAL STARCH under the trade name Hydrovance®.

In an other particular embodiment, the moisturizer agent is chosen from xylitol and its derivatives.

In an Other Particular Embodiment, the Moisturizer Agent is Chosen from the Phytantriol or TETRA-METHYL-3,7,11,15 TRI-HYDROXY-1,2,3 HEXA-DECANE.

The moisturizer agent may represent from 0.1% to 20% by weight of the total weight of the composition, preferably from 0.5 to 10%, and more preferably from 0.5 to 5% by weight of the total weight of the composition.

### 2. Desquamating Agents

The term “desquamating agent” means any compound capable of acting:
either directly on desquamation by promoting exfoliation, such as β-hydroxy acids (BHA), in particular salicylic acid and derivatives thereof (including 5-n-octanoylsalicylic acid, also known as capryloyl salicylic acid as the INCI name); α-hydroxy acids (AHA), such as glycolic acid, citric acid, lactic acid, tartaric acid, malic acid or mandelic acid; 8-hexadecene-1,16-dicarboxylic acid or 9-octadecenedioic acid; gentisic acid and derivatives thereof; oligofucoses; cinnamic acid; Saphora japonica extract; resveratrol, and certain jasmonic acid derivatives;
or on the enzymes involved in the desquamation or degradation of corneodesmosomes, glycosidases, stratum corneum chymotryptic enzyme (SCCE) or other proteases (trypsin, chymotrypsin-like). Mention may be made of aminosulfonic compounds and in particular 4-(2-hydroxyethyl)piperazine-1-propanesulfonic acid (HEPES); 2-oxothiazolidine-4-carboxylic acid (procysteine) derivatives; derivatives of α-amino acids of glycine type (as described in EP-0 852 949, and also sodium methyl glycine diacetate sold by BASF under the trade name Trilon M); honey; O-octanoyl-6-D-maltose and N-acetylglucosamine.

As other desquamating agents that may be used in the composition according to the invention, mention may be made of:
oligofructoses, EDTA and derivatives thereof, laminaria extracts, o-linoleyl-6D-glucose, (3-hydroxy-2-pentylcyclopentyl)acetic acid, glycerol trilactate, O-octanyl-6′-D-maltose, S-carboxymethylcysteine, siliceous derivatives of salicylate such as those described in patent EP 0 796 861, oligofucases such as those described in patent EP 0 218 200, 5-acyl salicylic acid salts, active agents with effects on transglutaminase, as in patent EP 0 899 330,
extract of the flowers of ficus Opuntia indica (Exfolactive® from Silab),
8-hexadecene-1,16-dicarboxylic acid,
esters of glucose and of vitamin F, and
mixtures thereof.

Preferred desquamating agents that may be mentioned include β-hydroxy acids such as 5-n-octanoyl salicylic acid; urea; glycolic acid, citric acid, lactic acid, tartaric acid, malic acid or mandelic acid; 4-(2-hydroxyethyl)piperazine-1-propanesulfonic acid (HEPES); extract of Saphora japonica; honey; N-acetyl glucosamine; sodium methyl glycine diacetate, and mixtures thereof.

Even more preferentially, a desquamating agent chosen from 5-n-octanoyl salicylic acid; urea; 4-(2-hydroxyethyl)piperazine-1-propanesulfonic acid (HEPES); extract of Saphora japonica; honey; N-acetyl glucosamine; sodium methyl glycine diacetate, and mixtures thereof, will be used in the compositions of the invention.

### 3. Agents for Improving the Barrier Function

As agents for improving the barrier function, mention may be made especially of arginine, an extract of Thermus thermophilus such as Venuceane® from Sederma, an extract of the rhizome of wild yam (Dioscorea villosa) such as Actigen Y® from Active Organics, plankton extracts, for instance Omega Plankton® from Secma, yeast extracts, for instance Relipidium® from Coletica, a chestnut extract such as Recoverine® from Silab, a cedar bud extract such as Gatuline Zen® from Gattefossé, sphingosines, for instance salicyloyl sphingosine sold under the name Phytosphingosine® SLC by the company Degussa, a mixture of xylitol, polyxylityl glycoside and xylitan, for instance Aquaxyl® from SEPPIC, extracts of Solanacea plants, for instance Lipidessence® from Coletica, omega-3 unsaturated oils such as musk rose oils, and mixtures thereof.

Mention may also be made especially of ceramides or derivatives thereof, in particular ceramides of type 2 (for instance N-oleoyldihydrosphingosine), of type 3 (for instance stearoyl-4-hydroxysphinganine, as the INCI name) and of type 5 (for instance N-2-hydroxypalmitoyldihydrosphingosine, having the INCI name: hydroxypalmitoyl sphinganine), sphingoid-based compounds, glycosphingolipids, phospholipids, cholesterol and derivatives thereof, phytosterols, essential fatty acids, diacylglycerol, 4-chromanone and chromone derivatives, petroleum jelly, lanolin, shea butter, cocoa butter, lanolin and PCA salts.

As preferred agents having a restructuring effect on the barrier function, mention will be made of an extract of Thermus thermophilus, an extract of wild yam rhizome (Dioscorea villosa), a yeast extract, a chestnut extract, a cedar bud extract, arginine, ceramides especially of type 3 and 5; and mixtures thereof.

### 4. Depigmenting Agents

Depigmenting agents that may especially be mentioned include vitamin C and derivatives thereof and especially vitamin CG, CP and 3-O ethyl vitamin C, alpha and beta arbutin, ferulic acid, lucinol and derivatives thereof, kojic acid, resorcinol and derivatives thereof, tranexamic acid and derivatives thereof, gentisic acid, homogentisate, methyl gentisate or homogentisate, dioic acid, calcium D-pantheteine sulfonate, lipoic acid, ellagic acid, vitamin B3, linoleic acid and derivatives thereof, ceramides and homologues thereof, plant derivatives, for instance camomile, bearberry, the aloe family (vera, ferox, bardensis), mulberry or skullcap; a kiwi fruit (Actinidia chinensis) juice sold by Gattefosse, an extract of Paeonia suffruticosa root, such as the product sold by the company Ichimaru Pharcos under the name Botanpi Liquid B®, an extract of brown sugar (Saccharum officinarum), such as the extract of molasses sold by the company Taiyo Kagaku under the name Molasses Liquid, without this list being exhaustive.

### 5. Antioxidants

Mention may be made especially of tocopherol and esters thereof, in particular tocopheryl acetate; ascorbic acid and derivatives thereof, in particular magnesium ascorbyl phosphate and ascorbyl glucoside; ferulic acid; serine; ellagic acid, polyphenols, tannins, tannic acid, epigallocatechins and natural extracts containing them, anthocyans, rosemary extracts, olive leaf extracts, for instance those from the company Silab, green tea extracts, resveratrol and derivatives thereof, ergothioneine, N-acetylcysteine, an extract of the brown alga Pelvetia caniculata, for instance Pelvetiane® from Secma, chlorogenic acid, biotin, chelating agents, such as BHT and BHA, N,N′-bis(3,4,5-trimethoxybenzyl)ethylenediamine and salts thereof; idebenone, plant extracts, for instance Pronalen Bioprotect™ from the company Provital; coenzyme Q10, bioflavonoids, SODs, phytanetriol, lignans, melatonin, pidolates, glutathione, caprylyl glycol, phloretin, Totarol™ or extract of Podocarpus totara containing Totarol (totara-8,11,13-trienol or 2-phenanthrenol, 4b,5,6,7,8,8a,9,10-octahydro-4b,8,8-trimethyl-1-(1-methylethyl)-; a jasmine extract such as the product sold by Silab under the name Helisun®; hesperitin laurate such as Flavagrum PEG® from the company Engelhard Lyon; an extract of Paeonia suffruticosa root, such as the product sold by the company Ichimaru Pharcos under the name Botanpi Liquid B®, an extract of lychee such as the extract of lychee pericarp sold by the company Cognis under the name Litchiderm LS 9704®, an extract of pomegranate fruit (Punica granatum), such as the product sold by the company Draco Natural Products.

Other anti-ageing agents that may be mentioned include DHEA and derivatives thereof, boswellic acid, rosemary extracts, carotenoids (β-carotene, zeaxanthin and lutein), cysteic acid, copper derivatives and jasmonic acid.

Preferred antioxidants that will especially be used include ferulic acid; serine; phloretin, an extract of pomegranate, biotin, chelating agents such as BHT, BHA, N,N′-bis(3,4,5-trimethoxybenzyl)ethylenediamine and salts thereof; caprylyl glycol, phloretin, Totarol™, a jasmine extract such as the product sold by Silab under the name Helisun®; hesperitin laurate such as Flavagrum PEG® from the company Engelhard Lyon; an extract of Paeonia suffruticosa root, such as the product sold by the company Ichimaru Pharcos under the name Botanpi Liquid B®.

6b. Dermo-Relaxing or Dermo-Decontracting Agents

Examples that may be mentioned include manganese gluconate and other salts, adenosine, alverine citrate and salts thereof, lysine, an extract of Iris pallida, a hexapeptide (Argeriline R from Lipotec) or sapogenins, for instance wild yam and the carbonyl amines described in patent application EP 1 484 052. Examples of sapogenins that may be mentioned include those described in patent application WO 02/47650, in particular wild yam, the diosgenin extracted especially from Dioscorea opposita or any extract naturally containing or containing after treatment one or more sapogenins (wild yam rhizome, agave leaf, which contains hecogenin and tigogenin, extracts of Liliacea plants and more particularly yucca or smilax containing smilagenin and sarsapogenin, or sarsaparilla) or Actigen Y from the company Actives Organics, or ginger.

Mention may also be made of DMAE (dimethyl MEA), extracts of sea fennel, of rockrose, of helichrysum, of aniseed, of paracress, and an extract of Acmella oleracea, for instance Gatuline® from Gattefossé. Preferred dermo-relaxing agents that will be mentioned include adenosine, manganese gluconate, wild yam, sea fennel, glycine and alverine.

### 7. Anti-Glycation Agents

The term “anti-glycation agent” means a compound that prevents and/or reduces the glycation of skin proteins, in particular dermal proteins such as collagen.

Anti-glycation agents that may especially be mentioned include extracts of plants of the Ericacea family, such as an extract of blueberry (Vaccinium angustifolium or Vaccinium myrtillus), for example the product sold under the name Blueberry Herbasol Extract PG by the company Cosmetochem, ergothioneine and derivatives thereof, hydroxystilbenes and derivatives thereof, such as resveratrol and 3,3′,5,5′-tetrahydroxystilbene (these anti-glycation agents are described in patent applications FR 2 802 425, FR 2 810 548, FR 2 796 278 and FR 2 802 420, respectively), dihydroxystilbenes and derivatives thereof, polypeptides of arginine and of lysine such as the product sold under the name Amadorine® by the company Solabia, carsinine hydrochloride (sold by Exsymol under the name Alistin®), an extract of Helianthus annuus, for instance Antiglyskin® from Silab, wine extracts such as the extract of powdered white wine on a maltodextrin support sold under the name Vin blanc déshydraté 2F by the company Givaudan, thioctic acid (or alpha-lipoic acid), a mixture of extract of bearberry and of marine glycogen, for instance Aglycal LS 8777® from Laboratoires Sérobiologiques, and an extract of black tea, for instance Kombuchka® from Sederma, and mixtures thereof.

Preferred anti-glycation agents that will be mentioned include extracts of blueberry (Vaccinium myrtillus) and extracts of black tea.

8. Agents for Stimulating the Synthesis of Dermal and/or Epidermal Macromolecules and/or for Preventing Their Degradation

Among the active agents for stimulating the dermal macromolecules or for preventing their degradation, mention may be made of those acting:
either on collagen synthesis, such as extracts of Centella asiatica, asiaticosides and derivatives thereof; ascorbic acid or vitamin C and derivatives thereof; synthetic peptides such as iamin, biopeptide CL or palmitoyl oligopeptide sold by the company Sederma; peptides extracted from plants, such as the soybean hydrolysate sold by the company Coletica under the trade name Phytokine®; rice peptides such as Nutripeptide® from Silab, methylsilanol mannuronate such as Algisium C® sold by Exsymol; plant hormones such as auxins and lignans; folic acid; and an extract of Medicago sativa (alfalfa) such as the product sold by Silab under the name Vitanol®; a peptide extract of hazelnut such as the product sold by the company Solabia under the name Nuteline C®; and arginine; or on the inhibition of collagen degradation, in particular agents acting on the inhibition of metalloproteases (MMP) more particularly such as MMP 1, 2, 3 and 9. Mention may be made of: retinoids and derivatives, extracts of Medicago sativa such as Vitanol® from Silab, an extract of Aphanizomenon flos-aquae (Cyanophyceae) sold under the name Lanablue® by Atrium Biotechnologies, oligopeptides and lipopeptides, lipoamino acids, the malt extract sold by the company Coletica under the trade name Collalift®; blueberry or rosemary extracts; lycopene; isoflavones, derivatives thereof or plant extracts containing them, in particular extracts of soybean (sold, for example, by the company Ichimaru Pharcos under the trade name Flavosterone SB®), of red clover, of flax or of kakkon; an extract of lychee; Dipalmitoyl Hydroxyproline sold by SEPPIC under the name Sepilift DPHP®: Baccharis genistelloides or Baccharine sold by Silab, an extract of moringa such as Arganyl LS 9781® from Cognis; the sage extract described in patent application FR-A-2 812 544 from the Labiatae family (Salvia officinalis from the company Flacksmann), an extract of rhododendron, a blueberry extract, and an extract of Vaccinium myrtillus such as those described in patent application FR-A-2 814 950;
or on the synthesis of molecules belonging to the elastin family (elastin and fibrillin), such as: retinol and derivatives, in particular retinyl palmitate; the extract of Saccharomyces cerevisiae sold by the company LSN under the trade name Cytovitin®; and the extract of the alga Macrocystis pyrifera sold by the company Secma under the trade name Kelpadelie®; a peptide extract of hazelnut such as the product sold by the company Solabia under the trade name Nuteline C®;
or on inhibition of elastin degradation, such as the peptide extract of seeds of Pisum sativum sold by the company LSN under the trade name Parelastyl®; heparinoids; and the N-acylamino acid compounds described in patent application WO 01/94381, such as {2-[acetyl(3-trifluoromethylphenyl)amino]-3-methyl-butyrylamino}acetic acid, also known as N—[N-acetyl, N′-(3-trifluoromethyl)phenyvalyl]glycine, or N-acetyl-N-[3-(trifluoromethyl)phenyl]valyl glycine or acetyl trifluoromethyl phenyl valylglycine, or an ester thereof with a C₁-C₆ alcohol; an extract of rice peptides such as Colhibin® from Pentapharm, or an extract of Phyllanthus emblica such as Emblica® from Rona;
or on the synthesis of glycosaminoglycans, such as the product of fermentation of milk with Lactobacillus vulgaris, sold by the company Brooks under the trade name Biomin Yoghurt®; the extract of the brown alga Padina pavonica sold by the company Alban Müller under the trade name HSP3®; the Saccharomyces cerevisiae extract available especially from the company Silab under the trade name Firmalift® or from the company LSN under the trade name Cytovitin®; an extract of Laminaria ochroleuca such as Laminaine® from Secma; essence of Mamaku from Lucas Meyer, and an extract of Cress (Odraline® from Silab);
or on the synthesis of fibronectin, such as the extract of the zooplankton Salina sold by the company Seporga under the trade name GP4G®; the yeast extract available especially from the company Alban Müller under the trade name Drieline®; and the palmitoyl pentapeptide sold by the company Sederma under the trade name Matrixyl®.

Among the active agents for stimulating epidermal macromolecules, such as fillagrin and keratins, mention may be made especially of the extract of lupin sold by the company Silab under the trade name Structurine®; the extract of Fagus sylvatica beech buds sold by the company Gattefosse under the trade name Gatuline® RC; and the extract of the zooplankton Salina sold by the company Seporga under the trade name GP4G®; the copper tripeptide from Procyte; a peptide extract of Voandzeia substerranea such as the product sold by the company Laboratoires Sérobiologiques under the trade name Filladyn LS 9397®.

Preferably, an active agent that stimulates the synthesis of dermal and/or epidermal macromolecules and/or that prevents their degradation, chosen from agents for stimulating the synthesis of glycosaminoglycans, agents for inhibiting elastin degradation, agents for stimulating fibronectin synthesis, agents for stimulating the synthesis of epidermal macromolecules, and mixtures thereof, will preferably be used.

Even more preferentially, an active agent that stimulates the synthesis of the glycosaminoglycans, chosen from an extract of the brown alga Padina pavonica, an extract of Saccharomyces cerevisiae, an extract of Laminaria ochroleuca, essence of Mamaku, and an extract of cress, and mixtures thereof, will even more preferentially be used.

As preferred active agents for stimulating the synthesis of dermal and/or epidermal macromolecules and/or for preventing their degradation, mention may be made of:

synthetic peptides such as iamin, the biopeptide CL or palmitoyloligopeptide sold by the company Sederma; peptides extracted from plants, such as the soybean hydrolysate sold by the company Coletica under the trade name Phytokine®; rice peptides such as Nutripeptide® from Silab, methylsilanol mannuronate such as Algisium C® sold by Exsymol; folic acid; an extract of Medicago sativa (alfalfa), such as the product sold by Silab under the name Vitanol®; a peptide extract of hazelnut, such as the product sold by the company Solabia under the name Nuteline C®; arginine; an extract of Aphanizomenon flos-aquae (Cyanophyceae) sold under the name Lanablue® by Atrium Biotechnologies, the malt extract sold by the company Coletica under the trade name Collalift®, lycopene; an extract of lychee; an extract of moringa such as Arganyl LS 9781® from Cognis; an extract of Vaccinium myrtillus such as those described in patent application FR-A-2 814 950; retinol and derivatives thereof, in particular retinyl palmitate; the extract of Saccharomyces cerevisiae sold by the company LSN under the trade name Cytovitin®; a peptide extract of hazelnut such as the product sold by the company Solabia under the name Nuteline C®; {2-[acetyl(3-trifluoromethylphenyl)amino]-3-methylbutyrylamino}-acetic acid, also known as N—[N-acetyl, N′-(3-trifluoromethyl)phenyvalyl]glycine, or N-acetyl-N-[3-(trifluoromethyl)phenyl]valyl glycine or acetyl trifluoromethyl phenyl valylglycine, or an ester thereof with a C₁-C₆ alcohol; an extract of rice peptides such as Colhibin® from Pentapharm, or an extract of Phyllanthus emblica such as Emblica® from Rona; the extract of the brown alga Padina pavonica sold by the company Alban Müller under the trade name HSP3®; the extract of Saccharomyces cerevisiae available especially from the company Silab under the trade name Firmalift® or from the company LSN under the trade name Cytovitin®; an extract of Laminaria ochroleuca such as Laminaine® from Secma; the essence of Mamaku from Lucas Meyer, the extract of lupin sold by the company Silab under the trade name Structurine®; the extract of Fagus sylvatica beech buds sold by the company Gattefosse under the trade name Gatuline®.
9. Agents for Stimulating Fibroblast or Keratinocyte Proliferation and/or Keratinocyte Differentiation

The agents for stimulating fibroblast proliferation that may be used in the composition according to the invention may be chosen, for example, from plant proteins or polypeptides, extracted especially from soybean (for example a soybean extract sold by the company LSN under the name Eleseryl SH-VEG 8® or sold by the company Silab under the trade name Raffermine®); an extract of hydrolysed soybean proteins such as Ridulisse® from Silab; and plant hormones such as gibberellins and cytokinins; a peptide extract of hazelnut such as the product sold by the company Solabia under the name Nuteline C®.

Preferably, an agent that promotes keratinocyte proliferation and/or differentiation will be used.

The agents for stimulating keratinocyte proliferation that may be used in the composition according to the invention especially comprise adenosine; phloroglucinol, the extract of Hydrangea macrophylla leaves, for instance Amacha Liquid E® from Ichimaru Pharcos, a yeast extract such as Stimoderm® from CLR; the extract of Larrea divaricata such as Capislow® from Sederma, mixtures of extract of papaya, of olive leaves and of lemon, such as Xyleine® from Vincience, the extract of Hydrangea macrophylla leaves, for instance Amacha Liquid E® from Ichimaru Pharcos, retinol and esters thereof, including retinyl palmitate, phloroglucinol, the nut cake extracts sold by the Gattefosse and the extracts of Solanum tuberosum such as Dermolectine® sold by Sederma.

Among the agents for stimulating keratinocyte differentiation are, for example, minerals such as calcium; sea fennel, a peptide extract of lupin, such as the product sold by the company Silab under the trade name Structurine®; sodium beta-sitosteryl sulfate, such as the product sold by the company Seporga under the trade name Phytocohesine®; and a water-soluble extract of corn, such as the product sold by the company Solabia under the trade name Phytovityl®; a peptide extract of Voandzeia substerranea such as the product sold by the company Laboratoires Sérobiologiques under the trade name Filladyn LS 9397®; and lignans such as secoisolariciresinol, and retinol and esters thereof, including retinyl palmitate.

As agents for stimulating keratinocyte proliferation and/or differentiation, mention may also be made of oestrogens such as oestradiol and homologues; cytokines.

As preferred active agents for stimulating fibroblast or keratinocyte proliferation and/or keratinocyte differentiation, mention will be made of plant proteins or polypeptides, extracted especially from soybean (for example a soybean extract sold by the company LSN under the name Eleseryl SH-VEG 8® or sold by the company Silab under the trade name Raffermine®); an extract of hydrolysed soybean proteins such as Ridulisse® from Silab; a peptide extract of hazelnut such as the product sold by the company Solabia under the name Nuteline C®; adenosine; phloroglucinol, a yeast extract such as Stimoderm® from CLR; a peptide extract of lupin such as the product sold by the company Silab under the trade name Structurine®; a water-soluble corn extract, such as the product sold by the company Solabia under the trade name Phytovityl®; a peptide extract of Voandzeia substerranea, such as the product sold by the company Laboratoires Sérobiologiques under the trade name Filladyn LS 9397®; retinol and esters thereof, including retinyl palmitate.

### 10. Agents for Promoting the Maturation of the Horny Envelope

Agents that participate in the maturation of the horny envelope, which becomes impaired with age and induces a decrease in transglutaminase activity, may be used in the compositions of the invention. Examples that may be mentioned include urea and derivatives thereof and in particular Hydrovance® from National Starch and the other active agents mentioned in L'Oréal patent application FR 2 877 220 (unpublished).

### 11. NO-Synthase Inhibitors

The agent with an inhibitory action on NO synthase may be chosen from OPCs (procyannidol oligomers); plant extracts of the species Vitis vinifera sold especially by the company Euromed under the name “Leucocyanidines de raisins extra”, or by the company Indena under the name Leucoselect®, or finally by the company Hansen under the name “Extrait de marc de raisin”; plant extracts of the species Olea europaea preferably obtained from olive tree leaves and sold especially by the company Vinyals in the form of a dry extract, or by the company Biologia & Technologia under the trade name Eurol BT; and plant extracts of the species Gingko biloba, preferably a dry aqueous extract of this plant sold by the company Beaufour under the trade name “Ginkgo biloba extrait standard”, and mixtures thereof.

### 12. Peripheral Benzodiazepine Receptor (PBR) Antagonists

Mention may be made, for example, of 1-(2-chlorophenyl)-N-(1-methylpropyl)-3-isoquinoline carboxamide; the compounds described in patent applications WO 03/030 937 and WO 03/068 753, pyridazino[4,5-b]indole-1-acetamide derivatives of general formula (VII) as described in document WO 00/44384.

### 13. Agents for Increasing the Activity of the Sebaceous Glands

Mention may be made, for example, of methyl dehydrojasmonate, hecogenin, hedione and O-linoleyl-6D-glucose, and mixtures thereof.

### 14. Agents for Stimulating the Energy Metabolism of Cells

The active agent for stimulating the energy metabolism of cells may be chosen, for example, from biotin, an extract of Saccharomyces cerevisiae such as Phosphovital® from Sederma, the mixture of sodium, manganese, zinc and magnesium salts of pyrrolidonecarboxylic acid, for instance Physiogenyl® from Solabia, a mixture of zinc, copper and magnesium gluconate, such as Sepitonic M3® from SEPPIC, and mixtures thereof; a beta-glucan derived from Saccharomyces cerevisiae, such as the product sold by the company Mibelle AG Biochemistry.

### 15. Tensioning Agents

The term “tensioning agent” that may be used according to the invention means compounds liable to have a tensioning effect, i.e. being able to make the skin taut.

According to the invention, the term “tensioning agent” generally means any polymer that is soluble or dispersible in water at a temperature ranging from 25° C. to 50° C. at a concentration of 7% by weight in water or at the maximum concentration at which a medium of uniform appearance is formed and producing at this concentration of 7% or at this maximum concentration in water a shrinkage of more than 15% in the test described below.

The maximum concentration at which a medium of uniform appearance forms is determined to within ±10% and preferably to within ±5%.

The expression “medium of uniform appearance” means a medium that does not contain any aggregates that are visible to the naked eye.

For the determination of the maximum concentration, the tensioning agent is gradually added to the water with deflocculating stirring at a temperature ranging from 25° C. to 50° C., and the mixture is then stirred for one hour. The mixture thus prepared is then examined after 24 hours to see if it is of uniform appearance (absence of aggregates visible to the naked eye).

The tensioning effect may be characterized by an in vitro shrinkage test.

A homogeneous mixture of the tensioning agent in water, at a concentration of 7% by weight or at the maximum concentration defined above, is prepared beforehand and as described previously.

30 μl of the homogeneous mixture are placed on a rectangular sample (10×40 mm, thus having an initial width L₀ of 10 mm) of elastomer with a modulus of 20 MPa and a thickness of 100 μm.

After drying for 3 hours at 22±3° C. and 40±10% relative humidity RH, the elastomer sample has a shrunken width, noted L₃ₕ, due to the tension exerted by the applied tensioning agent.

The tensioning effect (TE) of the polymer is then

$${ }^{‵} {TE}^{′} = ( {L}_{0} - {L}_{3 h} / {L}_{0} ) \times 100 as \%$$

$$with {L}_{0} = initial width 10 mm$$

$$and {L}_{3 h} = width after 3 hours of drying$$

The tensioning agent may be chosen from:

a) plant or animal proteins and hydrolysates thereof;
b) polysaccharides of natural origin;
c) mixed silicates;
d) colloidal particles of mineral fillers;
e) synthetic polymers;
and mixtures thereof.

A person skilled in the art will know how to choose, from the chemical categories listed above, the materials corresponding to the tensioning test as described below.

Mention may be made especially of:

(a) plant proteins and protein hydrolysates, in particular of corn, rye, wheat, buckwheat, sesame, spelt, pea, bean, lentil, soybean and lupin,
(b) polysaccharides of natural origin, especially (a) polyholosides, for example (i) in the form of starch derived especially from rice, corn, potato, cassaya, pea, wheat, oat, etc. or (ii) in the form of carrageenans, alginates, agars, gellans, cellulose polymers and pectins, advantageously as an aqueous dispersion of gel microparticles, and (b) latices consisting of shellac resin, sandarac gum, dammar resins, elemi gums, copal resins, cellulose derivatives, and mixtures thereof,
(c) mixed silicates, especially phyllosilicates and in particular Laponites,
(d) colloidal particles of mineral fillers with a number-average diameter of between 0.1 and 100 nm and preferably between 3 and 30 nm, and chosen, for example, from: silica, silica-alumina composites, cerium oxide, zirconium oxide, alumina, calcium carbonate, barium sulfate, calcium sulfate, zinc oxide and titanium dioxide. As silica-alumina composite colloidal particles that may be used in the compositions according to the invention, examples that may be mentioned include those sold by the company Grace under the names Ludox AM, Ludox AM-X 6021, Ludox HSA and Ludox TMA,
(e) synthetic polymers, such as polyurethane latices or acrylic-silicone latices, in particular those described in patent application EP-1 038 519, such as a polydimethylsiloxane grafted with propylthio(polymethyl acrylate), propylthio(polymethyl methacrylate) and propylthio(polymethacrylic acid), or alternatively a polydimethylsiloxane grafted with propylthio(polyiso-butyl methacrylate) and propylthio(polymethacrylic acid). Such grafted silicone polymers are especially sold by the company 3M under the trade names VS 80, VS 70 and LO21.

The tensioning agent will be present in the composition in an amount that is effective for obtaining the desired biological effect according to the invention.

By way of example, the tensioning agent may be included in the composition according to the invention in a content ranging from 0.01% to 30% by weight of active material and preferably from 1% to 30% by weight of active material relative to the total weight of the composition.

The term “active material” is intended to exclude the medium in which the tensioning agent may be dissolved or dispersed in its commercial form, for example in the case of dispersions of colloidal particles.

It is also possible, especially in order to complement and/or potentiate the effect of tensioning agents, to use agents that increase the expression of mechanoreceptors, such as agents that increase the expression of integrins.

An example that may be mentioned is an extract of rye seed, such as the product sold by Silab under the name Coheliss®.

### 16. Fat-Restructuring Agents

According to the invention, the term “fat-restructuring agents” means agents capable of stimulating lipogenesis and of promoting adipocyte differentiation, thus making it possible to prevent or slow down the wasting of fat contained in the skin supporting tissues, also known as “wasting of skin fat”.

The term “skin fat” means the network of fat cells that form the volumes on which the facial skin rests and is moulded.

These agents are intended:
to reduce the loss of skin density and/or the wasting of skin fat, in particular on the cheeks and around the eyes, and/or
to prevent the collapse and/or hollowing of the facial volumes, the loss of consistency of the skin and/or its maintenance, in particular on the cheeks and around the eyes, and/or
to improve the underlying volumes of the skin of the face and/or the neck, in particular on the cheeks, the oval of the face and around the eyes, and/or
to improve the density, springiness and maintenance of the skin, in particular on the cheeks, the oval of the face and around the eyes, and/or
   to remodel the facial features, in particular the oval of the face.

Examples of fat-restructuring agents that may especially be mentioned include an extract of black tea, such as the extract of fermented black tea sold by Sederma under the name Kombuchka®, and an extract of Artemisia abrotanum, such as the product sold by Silab under the name Pulpactyl®.

### 17. Slimming Agents

Slimming (lipolytic) agents that may especially be mentioned include theophylline and its derivatives, theobromine, acefylline, aminophylline, chloroethyl-theophylline, diprofylline, diniprophylline, etami-phylline and its derivatives, etofylline and proxyphylline; extracts of tea, of coffee, of guarana, of mate, of cola (Cola nitida) and especially the dry extract of guarana fruit (Paulina sorbilis) containing 8% to 10% caffeine; extracts of climbing ivy (Hedera helix), of arnica (Arnica montana L), of rosemary (Rosmarinus officinalis N), of marigold (Calendula officinalis), of sage (Salvia officinalis L), of ginseng (Panax ginseng), of St.-John's wort (Hypericum perforatum), of butcher's-broom (Ruscus aculeatus L), of meadowsweet (Filipendula ulmaria L), of orthosiphon (Orthosiphon stamincus Benth), of birch (Betula alba), of pumpwood and of argan tree, extracts of ginkgo biloba, extracts of horsetail, extracts of escin, extracts of cangzhu, extracts of Chrysanthellum indicum, extracts of diosgenin-rich Dioscorea plants or pure diosgenin or hecogenin and derivatives thereof, extracts of Ballota, extracts of Guioa, of Davallia, of Terminalia, of Barringtonia, of Trema or of Antirobia, the extract of bitter orange pips; an extract of husks of cocoa beans (Theobroma cacao) such as the product sold by Solabia under the name Caobromine®

### 18. Agents for Promoting the Cutaneous Microcirculation

The active agent acting on the cutaneous micro-circulation may be used for preventing dulling of the complexion and/or to improve the appearance of the area around the eyes, in particular to reduce the shadows around the eyes. It may be chosen, for example, from an extract of maritime pine bark, for instance Pycnogenol® from Biolandes, manganese gluconate (Givobio GMn® from SEPPIC), an extract of Ammi visnaga such as Visnadine from Indena, extract of lupin (Eclaline® from Silab), the protein coupling of hydrolysed wheat/palmitic acid with palmitic acid, such as Epaline 100 from Laboratoires Carilëne, the extract of bitter orange blossom (Remoduline® from Silab), vitamin P and derivatives thereof, for instance methyl-4 esculetol sodium monoethanoate sold under the name Permethol® by the company Sephytal, extracts of Ruscus, of common horse chestnut, of ivy, of ginseng and of melilot, caffeine, nicotinate and derivatives thereof, lysine and derivatives thereof, for instance Asparlyne® from Solabia, an extract of black tea such as Kombuchka from Sederma; rutin salts; an extract of the alga Corallina officinalis, such as the product sold by Codif; and mixtures thereof.

As preferred agents for promoting the cutaneous microcirculation, mention will be made of caffeine, an extract of bitter orange blossom, an extract of black tea, rutin salts and an extract of the alga Corallina officinalis.

### 19. Calmatives or Anti-Irritants

The term “calmative” means a compound that can reduce the sensation of stinging, itching or tautness of the skin.

As calmatives that may be used in the composition according to the invention, mention may be made of: procyannidol oligomers, vitamins E, C, B5 and B3, caffeine and derivatives thereof, pentacylic triterpenes and plant extracts containing them, β-glycyrrhetinic acid and salts or derivatives thereof (stearyl glycyrrhetate, 3-stearoyloxyglycyrrhetic acid or glycyrrhetinic acid monoglucuronide) and also plants containing them (e.g.: Glycyrrhiza glabra), oleanolic acid and salts thereof, ursolic acid and salts thereof, boswellic acid and salts thereof, betulinic acid and salts thereof, an extract of Paeonia suffruticosa and/or lactiflora, an extract of Laminaria saccharina, extracts of Centella asiatica, Canola oil, bisabolol, the phosphoric diester of vitamin E and C, for instance Sepivital EPC® from SEPPIC, camomile extracts, allantoin, omega-3 unsaturated oils such as musk rose oil, blackcurrant oil, Ecchium oil, fish oil or beauty-leaf oil, plankton extracts, capryloyl glycine, a mixture of water lily blossom extract and of palmitoylproline, such as the product sold under the name Seppicalm VG® by the company SEPPIC, an extract of Boswellia serrata, an extract of Centipeda cunninghami, such as the product sold under the name Cehami PF® by the company TRI-K Industries, an extract of sunflower seeds, in particular Hëlioxine® from Silab, an extract of Linum usitatissimum seeds, for instance Sensiline® from Silab, tocotrienols, piperonal, an extract of Epilobium angustifolium, such as the product sold under the name Canadian Willowherb Extract by the company Fytokem Products, Aloe vera, phytosterols, cornflower water, rose water, an extract of mint, in particular of mint leaves, for instance Calmiskin® from Silab, aniseed derivatives, filamentous bacteria, for instance Vitreoscilla filiformis as described in patent EP 761 204 and sold by Chimex under the name Mexoryl SBG®, an extract of rose petals, for instance Rose Flower Herbasol® extract from the company Cosmetochem, shea butter, a mixture of the waxy fraction of barley seeds obtained by supercritical CO₂, of shea butter and of argan oil, for instance Stimu-tex AS® from Pentapharm, alkaline-earth metal salts, especially of strontium, a fermented extract of Alteromonas sold under the name Abyssine® by the company Atrium Biotechnologies; spring water from the Vichy basin, such as waters originating from the Cëlestin, Chomel, Grande-Grille, Hôpital, Lucas and Parc sources, and preferably water from the Lucas source; an extract of Eperua falcata bark, such as the product sold by the company Cognis under the name Eperuline®; an extract of Paeonia suffruticosa root, such as the product sold by the company Ichimaru Pharcos under the name Botanpi Liquid B®; and mixtures thereof.

As preferred calmatives according to the invention, use will be made of:

β-glycyrrhetinic acid and salts or derivatives thereof (stearyl glycyrrhetate, 3-stearoyloxyglycyrrhetic acid or glycyrrhetinic acid monoglucuronide) and also plants containing them (e.g. Glycyrrhiza glabra); ursolic acid and salts thereof; extracts of Centella asiatica, Canola oil, bisabolol; camomile extracts, allantoin; a mixture of extract of water lily blossom and of palmitoylproline, such as the product sold under the name Seppicalm VG® by the company SEPPIC; Aloe vera, rose water, extract of mint, in particular of mint leaves, such as Calmiskin® from Silab, filamentous bacteria such as Vitreoscilla filiformis as described in patent EP 761 204 and sold by Chimex under the name Mexoryl SBG®, an extract of rose petals such as Rose Flower Herbasol® extract from the company Cosmetochem, shea butter, a fermented extract of Alteromonas sold under the name Abyssine® by the company Atrium Biotechnologies; spring water from the Vichy basin, such as waters originating from the Cëlestin, Chomel, Grande-Grille, Hôpital, Lucas and Parc sources, and preferably water from the Lucas source; an extract of Eperua falcata bark, such as the product sold by the company Cognis under the name Eperuline®; an extract of Paeonia suffruticosa root, such as the product sold by the company Ichimaru Pharcos under the name Botanpi Liquid B®; and mixtures thereof.

### 20. Sebo-Regulating or Anti-Seborrhoeic Agents

The term “sebo-regulating or anti-seborrhoeic agents” especially means agents capable of regulating the activity of the sebaceous glands.

Mention may be made especially of:
retinoic acid, benzoyl peroxide, sulfur, vitamin B6 (or pyridoxine), selenium chloride and sea fennel;
mixtures of extract of cinnamon, of tea and of octanoylglycine such as Sepicontrol A5 TEA® from SEPPIC;
the mixture of cinnamon, sarcosine and octanoylglycine sold especially by the company SEPPIC under the trade name Sepicontrol A5®;
zinc salts such as zinc gluconate, zinc pyrrolidonecarboxylate (or zinc pidolate), zinc lactate, zinc aspartate, zinc carboxylate, zinc salicylate and zinc cysteate;
copper derivatives and in particular copper pidolate such as Cuivridone® from Solabia;
extracts of plants of the species Arnica montana, Cinchona succirubra, Eugenia caryophyllata, Humulus lupulus, Hypericum perforatum, Mentha piperita, Rosmarinus officinalis, Salvia oficinalis and Thymus vulgaris, all sold, for example, by the company Maruzen;
extracts of meadowsweet (Spiraea ulmaria), such as the product sold under the name Sebonormine® by the company Silab;
extracts of the alga Laminaria saccharina, such as the product sold under the name Phlorogine® by the company Biotechmarine;
mixtures of extracts of salad burnet root (Sanguisorba officinalis/Poterium officinale), of ginger rhizomes (Zingiber officinalis) and of cinnamon bark (Cinnamomum cassia), such as the product sold under the name Sebustop® by the company Solabia;
linseed extracts, such as the product sold under the name Linumine® by the company Lucas Meyer;
Phellodendron extracts, such as those sold under the name Phellodendron extract BG by the company Maruzen or Oubaku liquid B by the company Ichimaru Pharcos;
mixtures of argan oil, of Serenoa serrulata (saw palmetto) extract and of sesame seed extract, such as the product sold under the name Regu SEB® by the company Pentapharm;
mixtures of extracts of willowherb, of Terminalia chebula, of nasturtium and of bioavailable zinc (microalgae), such as the product sold under the name Seborilys® by the company Green Tech;
extracts of Pygeum afrianum, such as the product sold under the name Pygeum afrianum sterolic lipid extract by the company Euromed;
extracts of Serenoa serrulata, such as the products sold under the name Viapure Sabal by the company Actives International or those sold by the company Euromed;
mixtures of extracts of plantain, of Berberis aquifolium and of sodium salicylate, such as the product sold under the name Seboclear® by the company Rahn;
clove extract, such as the product sold under the name Clove extract powder by the company Maruzen;
argan oil, such as the product sold under the name Lipofructyl® by Laboratoires Sérobiologiques;
lactic protein filtrates, such as the product sold under the name Normaseb® by the company Sederma;
extracts of the alga Laminaria, such as the product sold under the name Laminarghane® by the company Biotechmarine;
oligosaccharides of the alga Laminaria digitata, such as the product sold under the name Phycosaccharide AC by the company Codif;
sugar cane extracts, such as the product sold under the name Policosonol® by the company Sabinsa;
sulfonated shale oil, such as the product sold under the name Ichthyol Pale® by the company Ichthyol;
European meadowsweet (Spiraea ulmaria) extracts, such as the product sold under the name Cytobiol® Ulmaire by the company Libiol;
sebacic acid, especially sold in the form of a sodium polyacrylate gel under the name Sebosoft® by the company Sederma;
glucomannans extracted from konjac tuber and modified with alkylsulfonate chains, such as the product sold under the name Biopol Beta by the company Arch Chemical;
extracts of Sophora angustifolia, such as those sold under the name Sophora powder or Sophora extract by the company Bioland;
extracts of Cinchona succirubra bark, such as the product sold under the name Red Bark HS by the company Alban Muller;
extracts of Quillaja saponaria, such as the product sold under the name Panama wood HS by the company Alban Muller;
glycine grafted onto an undecylenic chain, such as the product sold under the name Lipacide UG OR by the company SEPPIC;
the mixture of oleanolic acid and of nordihydroguaiaretic acid, such as the product sold in the form of a gel under the name AC.Net by the company Sederma;
phthalimidoperoxyhexanoic acid;
tri(C₁₂-C₁₃)alkyl citrate sold under the name Cosmacol® ECI by the company Sasol; tri(C₁₄-C₁₅)alkyl citrate sold under the name Cosmacol® ECL by the company Sasol;
10-hydroxydecanoic acid, and especially mixtures of 10-hydroxydecanoic acid, of sebacic acid and of 1,10-decanediol, such as the product sold under the name Acnacidol® BG by the company Vincience; and
mixtures thereof.

Preferred anti-seborrhoeic active agents that may be mentioned include:
benzoyl peroxide and vitamin B6 (or pyridoxine),
zinc salts such as zinc gluconate, zinc pyrrolidonecarboxylate (or zinc pidolate), zinc lactate, zinc aspartate, zinc carboxylate, zinc salicylate and zinc cysteate;
meadowsweet (Spiraea ulmaria) extracts, such as the product sold under the name Sebonormine® by the company Silab;
extracts of the alga Laminaria saccharina, such as the product sold under the name Phlorogine® by the company Biotechmarine;
mixtures of extracts of salad burnet root (Sanguisorba officinalis/Poterium officinale), of ginger rhizomes (Zingiber officinalis) and of cinnamon bark (Cinnamomum cassia), such as the product sold under the name Sebustop® by the company Solabia;
clove extract, such as the product sold under the name Clove extract powder by the company Maruzen;
lactic protein filtrates, such as the product sold under the name Normaseb® by the company Sederma;
European meadowsweet (Spiraea ulmaria) extracts, such as the product sold under the name Cytobiol® Ulmaire by the company Libiol;
sebacic acid, especially sold in the form of a sodium polyacrylate gel under the name Sebosoft® by the company Sederma;
glycine grafted onto an undecylenic chain, such as the product sold under the name Lipacide UG OR by the company SEPPIC;
tri(C₁₂-C₁₃)alkyl citrate sold under the name Cosmacol® ECI by the company Sasol; tri(C₁₄-C₁₅)alkyl citrate sold under the name Cosmacol® ECL by the company Sasol;
10-hydroxydecanoic acid, and especially mixtures of 10-hydroxydecanoic acid, of sebacic acid and of 1,10-decanediol, such as the product sold under the name Acnacidol® BG by the company Vincience; and
mixtures thereof.

Preferentially, the anti-seborrhoeic active agent is chosen from:
zinc salts such as zinc gluconate, zinc pyrrolidonecarboxylate (or zinc pidolate), zinc lactate, zinc aspartate, zinc carboxylate, zinc salicylate and zinc cysteate; and preferably zinc pyrrolidonecarboxylate (or zinc pidolate) or zinc salicylate;
clove extract, such as the product sold under the name Clove extract powder by the company Maruzen;
glycine grafted onto an undecylenic chain, such as the product sold under the name Lipacide UG OR by the company SEPPIC;
tri(C₁₂-C₁₃)alkyl citrate sold under the name Cosmacol® ECI by the company Sasol; tri(C₁₄-C₁₅)alkyl citrate sold under the name Cosmacol® ECL by the company Sasol;
and mixtures thereof.

The anti-seborrhoeic active agent is, for example, present in a content ranging from 0.1% to 10% by weight, preferably from 0.1% to 5% by weight and preferentially from 0.5% to 3% by weight relative to the total weight of the composition.

### 21. Astringents

According to the invention, the term “astringents” means agents for combating the dilation of the sebaceous follicles.

As astringents that may be used in the composition according to the invention, mention may be made of extracts of mushroom pulp (Polyporus officinalis), for instance Laricyl LS8865® from Cognis, extracts of Terminalia catappa and Sambucus nigra, for instance Phytofirm LS9120® from Cognis, extracts of gall nut, for instance Tanlex VE® from Ichimaru Pharcos, aluminium hydroxychloride, centella extracts (e.g. Plantactiv centella from Cognis), dicetyl dimethylammonium chloride, for instance Varisoft 432 CG® from Degussa, common horsechestnut extracts, mallow extracts, witch-hazel extracts, sweet almond extracts, marshmallow root extracts and linseed extracts, for instance Almondermin LS 3380® from Cognis, burdock extracts, nettle extracts, birch extracts, horsetail extracts, camomile extracts, for instance those sold under the name Extrapone 9 special® by the company Symrise, skullcap extracts, European meadowsweet extracts (for example Cytobiol Ulmaire from Libiol), a mixture of extracts of white ginger, of horsetail, of nettle, of rosemary and of yucca, for instance Herb extract B1348 from Bell Flavors & Fragrances, extracts of acacia, of elm, of white willow, of cinnamon, of birch and of meadowsweet, Panama sapogenins, zinc phenolsulfonate from Interchemical, extracts of gentian, of cucumber and of walnut, the mixture of extracts of Ratanhia, of grapefruit, of gumweed and of oak gall, for instance Epilami® from Alban Muller.

As preferred astringents according to the invention, use will be made of skullcap extracts, European meadowsweet extracts, meadowsweet extracts, gentian extracts and burdock extracts, and mixtures thereof.

### 22. Cicatrizing Agents

Examples of cicatrizing agents that may especially be mentioned include:

allantoin, urea, certain amino acids, for instance hydroxyproline, arginine, and serine, and also extracts of white lily (for instance Phytélèene Lys 37EG 16295 from Indena), a yeast extract, for instance the cicatrizing agent LS LO/7225B from Laboratoires Sérobiologiques), tamanu oil, extract of Saccharomyces cerevisiae, for instance Biodynes® TRF® from Arch Chemical, oat extracts, chitosan and derivatives, for instance chitosan glutamate, carrot extracts, artemia extract, for instance GP4G® from Vincience, sodium acexamate, lavandin extracts, propolis extracts, ximeninic acid and salts thereof, rose hip oil, marigold extracts, for instance Souci Ami® Liposolible from Alban Muller, horsetail extracts, lemon peel extracts, for instance Herbasol® citron from Cosmetochem, helichrysum extracts, common yarrow extracts and folic acid.

As preferred cicatrizing agents according to the invention, use will be made of arginine, serine, folic acid, tamanu oil, sodium acexamate, horsetail extracts and helichrysum extracts, and mixtures thereof.

### 23. Anti-Inflammatory Agents

As particular anti-inflammatory agents that may be used according to the invention, mention may be made of cortisone, hydrocortisone, indomethacin, betamethasone, azelaic acid, acetaminophen, diclofenac, clobetasol propionate, folic acid; an extract of Eperua falcata bark, such as the product sold by the company Cognis under the name Eperuline®; an extract of Paeonia suffruticosa root, such as the product sold by the company Ichimaru Pharcos under the name Botanpi Liquid B®; and mixtures thereof.

Preferred anti-inflammatory agents that will be mentioned are azelaic acid, folic acid, an extract of Eperua falcata bark, such as the product sold by the company Cognis under the name Eperuline®; an extract of Paeonia suffruticosa root, such as the product sold by the company Ichimaru Pharcos under the name Botanpi Liquid B®; and mixtures thereof.

### 24. Antiacne Agents

In one advantageous aspect of the invention, the composition may also comprise at least one anti-acne active agent.

The term “anti-acne active agent” especially means any active agent that has effects on the specific flora of greasy skin, for instance Propionibacterium acnes (P. acnes). These effects may be bactericidal.

Antibactericidal active agents that may especially be mentioned include:
active agents and preserving agents with antimicrobial activity mentioned in patent application DE 103 24 567, which is incorporated into the present invention by reference,
Asiatic acid,
the monoethanolamine salt of 1-hydroxy-4-methyl 6-trimethylpentyl-2-pyridone (INCI name: piroctone olamine), sold especially under the name Octopirox® by the company Clariant;
citronellic acid, perillic acid (or 4-isopropenylcyclohex-1-enecarboxylic acid),
glyceryl 2-ethylhexyl ether (INCI name: ethylhexylglycerine), for example sold under the name Sensiva SC 50® by the company Shulke & Mayr,
glyceryl caprylate/caprate, for example sold under the name Capmul MCM® by the company Abitec;
sodium calcium phosphosilicate, especially sold under the names Bioactive Glasspowder® and Actysse Premier BG by the company Schott Glass;
silver-based particles, for example those sold under the name Metashine ME 2025 PS® by the company Nippon Sheet Glass;
hop cone extract (Humulus lupulus) obtained by supercritical CO₂ extraction, such as the product sold under the name HOP CO2-TO Extract® by the company Flavex Naturextrakte,
St.-John's Wort extract obtained by supercritical CO₂ extraction, such as the product sold under the name St.-John's Wort CO2-TO extract by the company Flavex Naturextrakte,
the mixture of extracts of roots of Scutellaria baicalensis, of Paeonia suffruticosa and Glycyrrhiza glabra, such as the product sold under the name BMB-CF® by the company Naturogin,
argan tree extract, for instance Argapure LS9710® from Cognis;
bearberry leaf extracts, for instance the product sold under the name Melfade-J by the company Pentapharm;
10-hydroxy-2-decanoic acid such as Acnacidol P® from Vincience, sodium ursolate, azelaic acid, diiodomethyl p-tolyl sulfone such as Amical Flowable® from Angus, malachite powder, zinc oxide such as Zincare® from Elementis GMBH, octadecenedioic acid such as Arlatone dioic DCA® from Uniqema; ellagic acid; 2,4,4′-trichloro-2′-hydroxydiphenyl ether (or triclosan), 1-(3′,4′-dichlorophenyl)-3-(4′-chloro-phenyl)urea (or triclocarban), 3,4,4′-trichloro-carbanilide, 3′,4′,5′-trichlorosalicylanilide, phenoxy-ethanol, phenoxypropanol, phenoxyisopropanol, hexamidine isethionate, metronidazole and salts thereof, miconazole and salts thereof, itraconazole, terconazole, econazole, ketoconazole, saperconazole, fluconazole, clotrimazole, butoconazole, oxiconazole, sulfaconazole, sulconazole, terbinafine, ciclopirox, ciclopiroxolamine, undecylenic acid and salts thereof, benzoyl peroxide, 3-hydroxybenzoic acid, 4-hydroxybenzoic acid, phytic acid, N-acetyl-L-cysteine, lipoic acid, azelaic acid and salts thereof, arachidonic acid, resorcinol, 3,4,4′-trichloro-carbanalide, octoxyglycerine or octoglycerine, octanoylglycine such as Lipacid C8G® from SEPPIC, caprylyl glycol, 10-hydroxy-2-decanoic acid, dichlorophenylimidazoldioxolane and derivatives thereof described in patent application WO 93/18743, iodopropynyl butylcarbamate, 3,7,11-trimethyldodeca-2,5,10-trienol or farnesol, phytosphingosines; quaternary ammonium salts, for instance cetyltrimethylammonium salts and cetylpyridinium salts, and
mixtures thereof.

Mention may also be made of certain surfactants with an antimicrobial effect, for instance sodium cocoamphoacetate or disodium diacetate such as Miranol C2M Conc. NP, betaines, for instance the cocoyl betaine Genagen KB from Clariant, sodium lauryl ether sulfate, for instance Emal 270 D from Kao, decyl glucoside, for instance Plantacare 2000 UP, branched C₁₂₋₁₃ dialkyl malates, for instance Cosmacol EMI, propylene glycol monoesters, for instance propylene glycol monolaurate, monocaprylate or monocaprate, lauryldimethylamine betaine, for instance Empigen BB/LS, and also polyquaternary ammoniums such as Quaternium-24 or Bardac 2050 from Lonza and those described in patent FR 0 108 283, and mixtures thereof.

As preferred antimicrobial agents, an agent chosen from caprylyl glycol, octoglycerine or octoxyglycerine, and 10-hydroxy-2-decanoic acid, and mixtures thereof, will be used in the compositions of the invention.

Other additional anti-acne active agents may be added to the abovementioned anti-acne active agents.

Mention may be made especially of active agents with bacterial anti-adhesion effects or agents that act on the biofilm of bacteria to prevent them from multiplying.

As agents for preventing and/or reducing the adhesion of microorganisms, mention may be made especially of: phytanetriol and derivatives thereof as described in patent application EP 1 529 523, plant oils such as wheatgerm oil, calendula oil, castor oil, olive oil, avocado oil, sweet almond oil, groundnut oil, jojoba oil, sesame seed oil, apricot kernel oil, sunflower oil and macadamia oil, described in patent EP 1 133 979, or certain surfactants such as disodium cocoamphodiacetate, oxyethylenated (7 EO) glyceryl cocoate, 18-hexadecenyl succinate, octoxyglyceryl palmitate, octoxyglyceryl behenate, dioctyl adipate, PPG-15 stearyl ether, and the branched C₁₂-C₁₃ dialkyl tartrates described in patent EP 1 129 694, and mixtures thereof.

In particular with regard to the propagation of P. acnes, or as active agents that act on the biofilm of bacteria to prevent them from proliferating, mention may be made of pentylene glycol, Nylon-66 (polyamide 66 fibres), rice bran oil, polyvinyl alcohol such as Celvol 540 PV Alcohol® from Celanese Chemical, rapeseed oil such as Akorex L® from Karlshamns, and fructose derivatives, and mixtures thereof.

The anti-acne active agent may be present in a content ranging from 0.01% to 10% by weight and preferably from 0.05% to 5% by weight relative to the total weight of the composition.

As a function of the nature and/or solubility of the abovementioned active agents, a person skilled in the art will know how to select the most suitable embodiment according to the invention.

As lipophilic active agents that may be used in the kit or at least one of the compositions of the invention, mention may be made especially of D-α-tocopherol, DL-α-tocopherol, D-α-tocopheryl acetate, DL-α-tocopheryl acetate, ascorbyl palmitate, vitamin F glycerides, D vitamins, vitamin D2, vitamin D3, retinol, retinol esters, retinyl palmitate, retinyl propionate, carotenes including β-carotene, D-panthenol, farnesol, farnesyl acetate, salicylic acid and derivatives thereof, for instance 5-n-octanoylsalicylic acid, α-hydroxy acid alkyl esters such as citric acid, lactic acid, glycolic acid, asiatic acid, madecassic acid, asiaticoside, the total extract of Centella asiatica, β-glycyrrhetinic acid, α-bisabolol, ceramides, for instance 2-oleoylamino-1,3-octadecane, phytanetriol, phospholipids of marine origin rich in polyunsaturated essential fatty acids, ethoxyquine, rosemary extract, balm extract, quercetin, extract of dried microalgae, essential oil of bergamot, octyl methoxycinnamate, butylmethoxydibenzoylmethane, octyl triazone, 3,5-di-tert-butyl-4-hydroxy-3-benzylidenecamphor, antibiotics, antifungal agents, anaesthetics, analgesics, antiseptics, antiviral agents, pesticides and herbicides, and mixtures thereof.

The cosmetic and/or dermatological active agents will be present in the kit or one of the compositions according to the invention in a content ranging from 0.001% to 20% relative to the total weight of the composition, preferably from 0.01% to 10%, even more preferentially from 0.5% to 5% and more preferably from 0.1% to 1% by weight relative to the total weight of the composition.

For “scrubbing” applications, the contents of cosmetic and/or dermatological active agents may range from 1% to 50% by weight relative to the total weight of the composition and preferably from 1% to 30% by weight relative to the total weight of the composition.

Scrubbing is a well-known means for improving the appearance and/or texture of the skin and/or the scalp, especially for improving the radiance and homogeneity of the complexion and/or for reducing the visible and/or tactile irregularities of the skin, and in particular for improving the surface appearance of the skin, for attenuating actinic lentigo, acne or chicken pox marks, and also for preventing, attenuating or combating the signs of ageing of the skin, and especially for smoothing out irregularities in the texture of the skin, such as wrinkles and fine lines.

It has the effect of removing a surface part of the skin to be treated (epidermis and possibly the upper layer of the dermis), via chemical methods.

Other Additional Ingredients

To complement and/or optimize the effects imparted by the cosmetic and/or dermatological active agents mentioned above on the keratin materials, it may be advantageous to incorporate into the compositions of the invention other additional ingredients.

In particular, these additional ingredients may impart an immediate visual effect that will be relayed by the biological effect of the active agents mentioned above.

They may also, via a mechanical action (e.g.: abrasive fillers), amplify the effect of the biological active agents mentioned above.

Thus, the kit or one of the compositions according to the invention may also comprise at least one agent chosen from matting agents, fillers with a soft-focus effect, fluorescers, agents for promoting the naturally pinkish coloration of the skin, abrasive fillers or exfoliants, and mixtures thereof.

### Matting Agents

The term “matting agent” means agents intended to make the skin visibly more matt and less shiny.

The matting effect of the agent and/or composition containing it may especially be evaluated using a gonioreflectometer, by measuring the ratio R between the specular reflection and the scattered reflection. A value of R of less than or equal to 2 generally reflects a matting effect.

The matting agent may especially be chosen from a rice starch or a corn starch, kaolinite, talc, a pumpkin seed extract, cellulose microbeads, plant fibres, synthetic fibres, in particular polyamide fibres, expanded acrylic copolymer microspheres, polyamide powders, silica powders, polytetrafluoroethylene powders, silicone resin powders, acrylic polymer powders, wax powders, polyethylene powders, powders of elastomeric crosslinked organopolysiloxane coated with silicone resin, talc/titanium dioxide/alumina/silica composite powders, amorphous mixed silicate powders, silicate particles and especially mixed silicate particles, and mixtures thereof.

Examples of matting agents that may especially be mentioned include:
rice or corn starch, in particular an aluminium starch octenyl succinate sold under the name Dry Flo® by the company National Starch;
kaolinite;
silicas;
talc;
a pumpkin seed extract as sold under the name Curbilene® by the company Indena;
cellulose microbeads as described in patent application EP 1 562 562;
fibres, such as silk fibre, cotton fibre, wool fibre, flax fibre, cellulose fibre extracted especially from wood, from vegetables or from algae, polyamide fibre (Nylon®), modified cellulose fibre, poly-p-phenyleneterephthamide fibre, acrylic fibre, polyolefin fibre, glass fibre, silica fibre, aramid fibre, carbon fibre, Teflon® fibre, insoluble collagen fibre, polyester fibre, polyvinyl chloride or polyvinylidene chloride fibre, polyvinyl alcohol fibre, polyacrylonitrile fibre, chitosan fibre, polyurethane fibre, polyethylene phthalate fibre, fibres formed from a mixture of polymers, resorbable synthetic fibres, and mixtures thereof described in patent application EP 1 151 742;
expanded acrylic copolymer microspheres such as those sold by the company EXPANCEL under the name Expancel 551®;
fillers with an optical effect as described in patent application FR 2 869 796, in particular:
   polyamide powders (Nylon®), for instance Nylon 12 particles of the Orgasol type from Arkema, with a mean size of 10 microns and a refractive index of 1.54,
   silica powders, for instance Silica beads SB150 from Miyoshi with a mean size of 5 microns and a refractive index of 1.45,
   polytetrafluoroethylene powders, for instance PTFE Ceridust 9205F from Clariant, with a mean size of 8 microns and a refractive index of 1.36,
   silicone resin powders, for instance the silicone resin Tospearl 145A from GE Silicone with a mean size of 4.5 microns and a refractive index of 1.41,
   acrylic copolymer powders, especially of polymethyl(meth)acrylate, for instance the PMMA particles Jurymer MBI from Nihon Junyoki, with a mean size of 8 microns and a refractive index of 1.49, or the Micropearl M100® and F 80 ED® particles from the company Matsumoto Yushi-Seiyaku,
   wax powders, for instance the paraffin wax particles Microease 114S from Micropowders, with a mean size of 7 microns and a refractive index of 1.54,
   polyethylene powders, especially comprising at least one ethylene/acrylic acid copolymer, and in particular consisting of ethylene/acrylic acid copolymers, for instance the particles Flobeads EA 209 from Sumitomo (with a mean size of 10 microns and a refractive index of 1.48),
   elastomeric crosslinked organopoly-siloxane powders coated with silicone resin, especially with silsesquioxane resin, as described, for example, in patent U.S. Pat. No. 5,538,793. Such elastomeric powders are sold under the names KSP-100, KSP-101, KSP-102, KSP-103, KSP-104 and KSP-105 by the company Shin-Etsu, and
   talc/titanium dioxide/alumina/silica composite powders such as those sold under the name Coverleaf® AR-80 by the company Catalyst & Chemicals,
   mixtures thereof,
   compounds that absorb and/or adsorb sebum as described in patent application FR 2 869 796. Mention may be made especially of:
   silica powders, for instance the porous silica microspheres sold under the name Silica Beads SB-700 sold by the company Myoshi, the products Sunsphere® H51, Sunsphere® H33 and Sunsphere® H53 sold by the company Asahi Glass; the polydimethylsiloxane-coated amorphous silica microspheres sold under the name SA Sunsphere® H-33 and SA Sunsphere® H-53 sold by the company Asahi Glass;
   amorphous mixed silicate powders, especially of aluminium and magnesium, for instance the product sold under the name Neusilin UFL2 by the company Sumitomo;
   polyamide (Nylon®) powders, for instance Orgasol® 4000 sold by the company Arkema, and
   acrylic polymer powders, especially of polymethyl methacrylate, for instance Covabead® LH85 sold by the company Wackherr; of polymethyl methacrylate/ethylene glycol dimethacrylate, for instance Dow Corning 5640 Microsponge® Skin Oil Adsorber sold by the company Dow Corning, or Ganzpearl® GMP-0820 sold by the company Ganz Chemical; of polyallyl methacrylate/ethylene glycol dimethacrylate, for instance Poly-Pore® L200 or Poly-Pore® E200 sold by the company Amcol; of ethylene glycol dimethacrylate/lauryl methacrylate copolymer, for instance Polytrap® 6603 sold by the company Dow Corning;
   silicate particles, such as alumina silicate;
   mixed silicate particles, such as:
   magnesium aluminium silicate particles, such as saponite or hydrated magnesium aluminium silicate with a sodium sulfate sold under the trade name Sumecton® by the company Kunimine;
      the magnesium silicate, hydroxyethylcellulose, black cumin oil, marrow oil and phospholipids complex or Matipure® from Lucas Meyer, and
   mixtures thereof.

Preferred matting agents that may be used according to the invention include a pumpkin seed extract, a rice or corn starch, kaolinite, silicas, talc, polyamide powders, polyethylene powders, acrylic copolymer powders, expanded acrylic copolymer microspheres, silicone resin microbeads and mixed silicate particles, and mixtures thereof.

Fillers with a Soft-Focus Effect

These fillers may be any material capable of modifying and hiding wrinkles by virtue of their intrinsic physical properties. These fillers may especially modify wrinkles via a tensioning effect, a covering effect or a soft-focus effect.

Examples of fillers that may be given include the following compounds:
porous silica microparticles, for instance the silica beads SB150 and SB700 from Miyoshi with a mean size of 5 μm; the series-H Sunspheres from Asahi Glass, for instance Sunspheres H33, H51 with respective sizes of 3.5 and 5 μm;
hollow hemispherical silicone resin particles such as NLK 500®, NLK 506® and NLK 510® from Takemoto Oil and Fat, especially described in EP-A-1 579 849;
silicone resin powders, for instance the silicone resin Tospearl 145A from GE Silicone, with a mean size of 4.5 μm;
acrylic copolymer powders, especially of polymethyl (meth)acrylate, for instance the PMMA particles Jurymer MBI® from Nihon Junyoki, with a mean size of 8 μm, the hollow PMMA spheres sold under the name Covabead® LH85 by the company Wackherr, and vinylidene/acrylo-nitrile/methylene methacrylate expanded microspheres sold under the name Expancel®;
wax powders, for instance the paraffin wax particles MicroEase® 114S from MicroPowders, with a mean size of 7 μm;
polyethylene powders, especially comprising at least one ethylene/acrylic acid copolymer, and in particular consisting of ethylene/acrylic acid copolymers, for instance the Flobeads® EA 209 particles from Sumitomo, with a mean size of 10 μm;
crosslinked elastomeric organopolysiloxane powders coated with silicone resin and especially with silsesquioxane resin, under the names KSP-100®, KSP-101®, KSP-102®, KSP-103®, KSP-104® and KSP-105® by the company Shin-Etsu;
talc/titanium dioxide/alumina/silica composite powders, for instance those sold under the name Coverleaf AR-80® by the company Catalyst & Chemicals;
talc, mica, kaolin, lauryl glycine, starch powders crosslinked with octenyl succinate anhydride, boron nitride, polytetrafluoroethylene powders, precipitated calcium carbonate, magnesium carbonate, magnesium hydrogen carbonate, barium sulfate, hydroxyapatite, calcium silicate, cerium dioxide and glass or ceramic microcapsules;
hydrophilic or hydrophobic, synthetic or unnatural, mineral or organic fillers such as silk fibres, cotton fibres, wool fibres, flax fibres, cellulose fibres extracted especially from wood, vegetables or algae, Polyamides (Nylon®) fibres, modified cellulose fibres, poly-p-terephthamide fibres, acrylic fibres, polyolefin fibres, glass fibres, silica fibres, aramid fibres, carbon fibres, polytetrafluoroethylene (Teflon®) fibres, insoluble collagen fibres, polyester fibres, polyvinyl chloride fibres, polyvinylidene chloride fibres, polyvinyl alcohol fibres, polyacrylonitriles fibres, chitosan fibres, polyurethane fibres, polyethylene phthalate fibres, fibres formed from a mixture of polymers, resorbable synthetic fibres, and mixtures thereof described in patent application EP 1 151 742;
spherical elastomeric crosslinked silicones, for instance Trefil E-505C® or E-506C® from Dow Corning;
abrasive fillers, which, via a mechanical effect, smooth out the skin microrelief, such as abrasive silica, for instance Abrasif SP® from Semanez or nutshell powders (for example of apricot or walnut, from Cosmëtochem).

The fillers with an effect on the signs of ageing are especially chosen from porous silica microparticles, hollow hemispherical silicones, silicone resin powders, acrylic copolymer powders, polyethylene powders, crosslinked elastomeric organopolysiloxanes powders coated with silicone resin, talc/titanium dioxide/alumina/silica composite powders, precipitated calcium carbonate, magnesium carbonate, magnesium hydrogen carbonate, barium sulfate, hydroxyapatite, calcium silicate, cerium dioxide, glass or ceramic microcapsules, and silk fibres or cotton fibres, and mixtures thereof.

The filler may be a soft-focus filler.

The term “soft-focus” filler means a filler which in addition gives the complexion transparency and a hazy effect. Preferably, the soft-focus fillers have a mean particle size of less than or equal to 15 microns. These particles may be in any form and in particular may be spherical or non-spherical. These fillers are more preferably non-spherical.

The soft-focus fillers may be chosen from silica and silicate powders, especially alumina powder, powders of polymethyl methacrylate (PMMA) type, talc, silica/TiO₂ or silica/zinc oxide composites, polyethylene powders, starch powders, polyamide powders, styrene/acrylic copolymer powders and silicone elastomers, and mixtures thereof.

Mention may be made in particular of talc with a number-average size of less than or equal to 3 microns, for example talc with a number-average size of 1.8 microns and especially the product sold under the trade name Talc P3® by the company Nippon Talc, Nylon 12 powder, especially the product sold under the name Orgasol 2002 Extra D Nat Cos® by the company Atochem, silica particles 1% to 2% surface-treated with a mineral wax (INCI name: hydrated silica (and) paraffin) such as the products sold by the company Degussa, amorphous silica microspheres, such as the products sold under the name Sunsphere, for example of reference H-53® by the company Asahi Glass, and silica microbeads such as those sold under the name SB-700 or SB-150® by the company Miyoshi, this list not being limiting.

The concentration of these fillers with an effect on the signs of ageing in the compositions according to the invention may be between 0.1% and 40%, or even between 0.1% and 20% by weight, relative to the total weight of the composition.

### Fluorescers

The term “fluorescer” means a substance which, under the effect of ultraviolet rays and/or visible light, re-emits in the visible region the portion of light that it has absorbed under the same colour as that which it naturally reflects. The naturally reflected colour is thus reinforced by the re-emitted colour and appears extremely bright.

Examples that may be mentioned include coloured polyamide and/or formaldehyde/benzoguanamine and/or melamine/formaldehyde/sulfonamide resins, from coloured aminotriazine/formaldehyde/sulfonamide co-condensates and/or from metallized polyester flakes and/or mixtures thereof. These fluorescent pigments may also be present in the form of aqueous dispersions of fluorescent pigments.

Mention may also be made of the pink-coloured fluorescent aminotriazine/formaldehyde/sulfonamide co-condensate with a mean particle size of 3-4 microns sold under the trade name “Fiesta Astral Pink FEX-1” and the blue-coloured fluorescent aminotriazine/formaldehyde/sulfonamide co-condensate with a mean particle size of 3-4.5 microns sold under the trade name “Fiesta Comet Blue FTX-60” by the company Swada, or alternatively the yellow-coloured benzoguanamine/formaldehyde resin covered with formaldehyde/urea resin sold under the trade name “FB-205 Yellow” and the red-coloured benzoguanamine/formaldehyde resin covered with formaldehyde/urea resin sold under the trade name “FB-400 Orange Red” by the company UK Seung Chemical, and the orange-coloured polyamide resin sold under the trade name “Flare 911 Orange 4” by the company Sterling Industrial Colors.

The fluorescent substances are preferably present in the composition in a content ranging from 0.1% to 20%, preferably from 0.1% to 15% and more preferably from 0.5% to 3% by weight relative to the total weight of the composition.

When the organic fluorescent substances are white, they are also known as optical brighteners.

The optical brightener has the effect of intensifying the radiance and reviving the shades of cosmetic compositions comprising them on application to the skin.

Among the optical brighteners that may be mentioned more particularly are stilbene derivatives, in particular polystyrylstilbenes and triazinestilbenes, coumarin derivatives, in particular hydroxycoumarins and aminocoumarins, oxazole, benzoxazole, imidazole, triazole and pyrazoline derivatives, pyrene derivatives and porphyrin derivatives, and/or mixtures thereof.

Such compounds are available, for example, under the trade names Tinopal SOP® and Uvitex OB® from the company Ciba Geigy.

The optical brighteners preferentially used are sodium 4,4′-bis[(4,6-dianilino-1,3,5-triazin-2-yl)amino]-stilbene-2,2′-disulfonate, 2,5-thiophenediylbis(5-tert-butyl-1,3-benzoxazole) and disodium 4,4′-distyrylbi-phenylsulfonate, and/or mixtures thereof.

### Agents for Promoting the Naturally Pinkish Coloration of the Skin

Mention may be made especially of:
a self-tanning agent, i.e. an agent which, when applied to the skin, especially to the face, can produce a tan effect that is more or less similar in appearance to that which may result from prolonged exposure to the sun (natural tan) or under a UV lamp;
an additional colouring agent, i.e. any compound that has particular affinity for the skin, which allows it to give the skin a lasting, non-covering coloration (i.e. that does not have a tendency to opacity the skin) and that is not removed either with water or using a solvent, and that withstands both rubbing and washing with a solution containing surfactants. Such a lasting coloration is thus distinguished from the superficial and transient coloration provided, for example, by a makeup pigment;
   and mixtures thereof.

Examples of self-tanning agents that may especially be mentioned include:
dihydroxyacetone (DHA),
erythrulose, and
the combination of a catalytic system formed from:
manganese and/or zinc oxide salts, and
alkali metal and/or alkaline-earth metal hydrogen carbonates.

The self-tanning agents are generally chosen from monocarbonyl or polycarbonyl compounds, for instance isatin, alloxan, ninhydrin, glyceraldehyde, mesotartaric aldehyde, glutaraldehyde, erythrulose, pyrazoline-4,5-dione derivatives as described in patent application FR 2 466 492 and WO 97/35842, dihydroxy-acetone (DHA) and 4,4-dihydroxypyrazolin-5-one derivatives as described in patent application EP 903 342. DHA will preferably be used.

The DHA may be used in free and/or encapsulated form, for example in lipid vesicles such as liposomes, especially described in patent application WO 97/25970.

In general, the self-tanning agent is present in an amount ranging from 0.01% to 20% by weight and preferably in an amount of between 0.1% and 10% of the total weight of the composition.

Examples of additional colouring agents that may be mentioned include plant extracts, for instance sorghum extracts obtained from the whole plant or from the stems, seeds or leaves of the genus Sorghum. The preferred Sorghum species are chosen from Sorghum bicolor, Sorghum caudatum, Sorghum nervosum, Sorghum durra, Sorghum vulgare and Sorghum species in combination with Colletotrichum graminicola, for instance those described in patent application FR 02/00251.

Other dyes that allow modification of the colour produced by the self-tanning agent may also be used.

These dyes may be chosen from synthetic or natural direct dyes.

These dyes may be chosen, for example, from red or orange dyes of the fluorane type such as those described in patent application FR 2 840 806. Mention may be made, for example, of the following dyes:
tetrabromofluoresceine or eosin known under the CTFA name: CI-45380 or Red 21;
phloxin B known under the CTFA name: CI-45410 or Red 27;
diiodofluoresceine known under the CTFA name: CI-45425 or Orange 10;
dibromofluoresceine known under the CTFA name: CI-45370 or Orange 5;
the sodium salt of tetrabromofluoresceine known under
the CTFA name: CI-45380 (Na salt) or Red 22;
the sodium salt of phloxin B known under the CTFA name: CI-45410 (Na salt) or Red 28;
the sodium salt of diiodofluoresceine known under the CTFA name: CI-45425 (Na salt) or Orange 11;
erythrosine known under the CTFA name: CI-45430 or Acid Red 51;
phloxin known under the CTFA name: CI-45405 or Acid Red 98.

These dyes may also be chosen from anthraquinones, caramel, carmine, carbon black, azulene blues, methoxalene, trioxalene, guajazulene, chamuzulene, Bengal rose, cosin 10B, cyanosin and daphinin.

These dyes may also be chosen from indole derivatives, for instance the monohydroxyindoles as described in patent FR 2 651 126 (i.e.: 4-, 5-, 6- or 7-hydroxy-indole) or the dihydroxyindoles as described in patent EP-B-0 425 324 (i.e.: 5,6-dihydroxyindole, 2-methyl-5,6-dihydroxyindole, 3-methyl-5,6-dihydroxyindole or 2,3-dimethyl-5,6-dihydroxyindole).

### Abrasive Fillers or Exfoliants

As exfoliants that may be used in rinse-out compositions according to the invention, examples that may be mentioned include exfoliants for scrubbing particles of mineral, plant or organic origin. Thus, polyethylene beads or powder, Nylon powder, polyvinyl chloride powder, pumice powder, ground apricot kernel or walnut husk, sawdust, glass beads and alumina, and mixtures thereof, may be used, for example.

Mention may also be made of Exfogreen® from Solabia (bamboo extract), extracts of strawberry akenes (Strawberry Akenes from Greentech), peach kernel powder, apricot kernel powder, and finally, in the field of plant powders with an abrasive effect, mention may be made of cranberry kernel powder.

As abrasive fillers or exfoliants that are preferred according to the invention, mention will be made of peach kernel powder, apricot kernel powder, cranberry kernel powder, strawberry akene extracts and bamboo extracts.

The additional ingredients(s) used in the kit or one of the compositions according to the invention may represent from 0.0001% to 20%, preferably from 0.01% to 10% and better still from 0.01% to 10% by weight relative to the total weight of the composition.

According to one particular mode, the kit or one of the compositions according to the invention contains at least urea or a derivative thereof, such as Hydrovance® from National Starch.

According to another mode, the kit or one of the compositions according to the invention contains at least hyaluronic acid or hyaluronic acid spheres.

According to another mode, the kit or one of the compositions according to the invention contains at least acrylic acid homopolymers, for instance Lipidure-HM® from NOF Corporation.

According to another mode, the kit or one of the compositions according to the invention contains at least one C-glycoside derivative such as those described in patent application WO 02/051 828 and in particular C-β-D-xylopyranoside-2-hydroxypropane in the form of a solution containing 30% by weight of active material in a water/propylene glycol mixture (60/40% by weight), such as the product sold by Chimex under the trade name Mexoryl SBB®.

According to another mode, the kit or one of the compositions according to the invention contains at least spheres of collagen and of chondroitin sulfate of marine origin (Atelocollagen) sold by the company Engelhard Lyon under the name marine filling spheres.

According to another mode, the kit or one of the compositions according to the invention contains at least kojic acid.

According to another mode, the kit or one of the compositions according to the invention contains at least ellagic acid.

According to another mode, the kit or one of the compositions according to the invention contains at least 5-N-octanoylsalicylic acid, also known as capryloyl salicylic acid as the INCI name.

According to another mode, the kit or one of the compositions according to the invention contains at least 8-hexadecene-1,16-dicarboxylic acid or 9-octadecenedioic acid and derivatives thereof.

According to another mode, the kit or one of the compositions according to the invention contains at least 4-(2-hydroxyethyl)piperazine-1-propanesulfonic acid (HEPES).

According to another mode, the kit or one of the compositions according to the invention contains at least 2-oxothiazolidine-4-carboxylic acid (procysteine) and derivatives thereof.

The according to another mode, the kit or one of the compositions according to the invention contains at least {2-[acetyl(3-trifluoromethylphenyl)amino]-3-methylbutyrylamino}acetic acid, also known as N—[N-acetyl, N′-(3-trifluoromethyl)phenylvalyl]glycine, or N-acetyl-N-[3-(trifluoromethyl)phenyl]valylglycine or acetyl trifluoromethyl phenyl valylglycine.

According to another mode, the kit or one of the compositions according to the invention contains at least one sphingosine, for instance salicyloyl sphingosine sold under the name Phytosphingosine® SLC by the company Degussa.

According to another mode, the kit or one of the compositions according to the invention contains at least one ceramide or derivative, in particular ceramides of type 2 (for instance N-oleoyldihydrosphingosine, as the INCI name) and of type 5 (for instance N-2-hydroxypalmitoyldihydrosphingosine, having the INCI name: hydroxypalmitoyl sphinganine).

According to another mode, the kit or one of the compositions according to the invention contains at least one ascorbyl glucoside (vitamin CG).

According to another mode, the kit or one of the compositions according to the invention contains at least vitamin B3.

According to another mode, the kit or one of the compositions according to the invention contains at least biotin.

According to another mode, the kit or one of the compositions according to the invention contains at least caprylyl glycol.

According to another mode, the kit or one of the compositions according to the invention contains at least phloretin.

According to another mode, the kit or one of the compositions according to the invention contains at least Totarol™ or extract of Podocarpus totara containing Totarol (totara-8,11,13-trienol or 2-phenanthrenol, 4b,5,6,7,8,8a,9,10-octahydro-4b,8,8-trimethyl-1-(1-methylethyl)-.

According to another mode, the kit or one of the compositions according to the invention contains at least one extract of pomegranate.

According to another mode, the kit or one of the compositions according to the invention contains at least ferulic acid.

According to another mode, the kit or one of the compositions according to the invention contains at least adenosine.

According to another mode, the kit or one of the compositions according to the invention contains at least ginger.

According to another mode, the kit or one of the compositions according to the invention contains at least manganese gluconate.

According to another mode, the kit or one of the compositions according to the invention contains at least one sapogenin or a natural extract containing it, in particular an extract of wild yam.

According to another mode, the kit or one of the compositions according to the invention contains at least one extract of sea fennel.

According to another mode, the kit or one of the compositions according to the invention contains at least retinol or a derivative thereof, in particular retinyl palmitate.

According to another mode, the kit or one of the compositions according to the invention contains at least one extract of blueberry (Vaccinium angustifolium or Vaccinium myrtillus).

According to another mode, the kit or one of the compositions according to the invention contains at least one extract of black tea, for instance Kombuchka.

According to another mode, the kit or one of the compositions according to the invention contains at least methylsilanol mannuronate.

According to another mode, the kit or one of the compositions according to the invention contains at least folic acid or vitamin B9.

According to another mode, the kit or one of the compositions according to the invention contains at least lycopene.

According to another mode, the kit or one of the compositions according to the invention contains at least one yeast extract, in particular an extract of Saccharomyces cerevisiae.

According to another mode, the kit or one of the compositions according to the invention contains at least one extract of lupin sold by the company Silab under the trade name Structurine®.

According to another mode, the kit or one of the compositions according to the invention contains at least one extract of hydrolysed soybean proteins.

According to another mode, the kit or one of the compositions according to the invention contains at least rice proteins or peptides.

According to one particular mode, the kit or one of the compositions according to the invention contains at least arginine and/or serine.

According to another particular mode, the kit or one of the compositions according to the invention contains at least omega-3 and/or omega-5 unsaturated oils such as musk rose oils.

According to another particular mode, the kit or one of the compositions according to the invention contains at lest one extract of Artemisia abrotanum, such as Pulpactyl from Silab.

According to another mode, the kit or one of the compositions according to the invention contains at least one silica-alumina composite colloidal particles, for example those sold by the company Grace under the names Ludox AM, Ludox AM-X 6021, Ludox HSA and Ludox TMA.

According to another mode, the kit or one of the compositions according to the invention contains at least one grafted silicone polymer such as those sold especially by the company 3M under the trade names VS 80, VS 70 or LO21.

According to another mode, the kit or one of the compositions according to the invention contains at least one extract of rye seed, such as the product sold by Silab under the name Coheliss®.

According to another mode, the kit or one of the compositions according to the invention contains at least caffeine.

According to another mode, the kit or one of the compositions contains at least ginseng.

According to another mode, the kit or one of the compositions according to the invention contains at least ginkgo.

According to another mode, the kit or one of the compositions according to the invention contains at least ruscus.

According to another mode, the kit or one of the compositions according to the invention contains at least escin.

According to another mode, the kit or one of the compositions according to the invention contains at least one extract of mint, in particular of mint leaves, for instance Calmiskin® from Silab.

According to another mode, the kit or one of the compositions according to the invention contains at least one extract of filamentous bacteria, for instance Vitreoscilla filiformis as described in patent EP 761 204 and sold by Chimex under the name Mexoryl SBG®.

According to another mode, the kit or one of the compositions according to the invention contains at least one extract of rose petals, for instance Rose Flower Herbasol® extract from the company Cosmetochem.

According to another mode, the kit or one of the compositions according to the invention contains at least one fermented extract of Alteromonas sold under the name Abyssine®.

According to another mode, the kit or one of the compositions according to the invention contains at least one spring water from the Vichy basin, such as waters originating from the Cëlestin, Chomel, Grande-Grille, Hôpital, Lucas and Parc sources, and preferably water from the Lucas source.

According to another mode, the kit or one of the compositions according to the invention contains at least one extract of linseed, such as the product sold under the name Linumine® by the company Lucas Meyer.

According to another mode, the kit or one of the compositions according to the invention contains at least hollow hemispherical particles of silicone resins, for instance NLK 500®, NLK 506® and NLK 510® from Takemoto Oil and Fat, described especially in EP-A-1 579 849.

According to another mode, the kit or one of the compositions according to the invention contains at least one emulsifying silicone elastomer such as those sold under the names KSG-210, KSG-310, KSG-320, KSG-330, KSG-440, KSG-710, KSG-830 and KSG-840 by the company Shin-Etsu.

According to another mode, the kit or one of the compositions according to the invention contains at least one silicone elastomer, such as an elastomeric organopolysiloxane, which is preferably at least partially crosslinked (e.g.: KSG). According to another mode, the kit or one of the compositions according to the invention contains at least DHA.

Physiologically Acceptable Medium

As stated previously, the compositions according to the invention comprise a physiologically acceptable medium, i.e. a non-toxic medium that may be applied to human keratin materials and which is of pleasant appearance, odour and feel.

The compositions according to the invention advantageously contain at least one liquid fatty phase.

The compositions according to the invention may be in the form of anhydrous compositions or emulsions.

When the compositions are in anhydrous form, they may be in the form of liquids, soft pastes, sticks, compact or cast products, or alternatively free powders.

Advantageously, they may be in the form of emulsion.

The compositions according to the invention may advantageously be in the form of an emulsion obtained by dispersing an aqueous phase in a fatty phase (W/O) or a fatty phase in an aqueous phase (O/W), of liquid or semi-liquid consistency of the milk type, or of soft, semi-solid or solid consistency of the cream or gel type, or alternatively in the form of a multiple emulsion (W/O/W or O/W/O). These compositions are prepared according to the usual methods.

The compositions of this type may be in the form of a care or makeup product for the face and/or the body, and may be conditioned, for example, in the form of cream in a jar or of fluid in tube or in a pump-dispenser bottles.

The emulsions will generally be chosen from direct emulsions obtained by dispersing a fatty phase in an aqueous phase (O/W), inverse emulsions obtained by dispersing an aqueous phase in a fatty phase (W/O) or multiple emulsions obtained by dispersing an inverse emulsion in an aqueous phase (W/O/W). These compositions are prepared according to the usual methods.

More particularly, they are direct emulsions obtained by dispersing a fatty phase in an aqueous phase (O/W).

Compounds X and Y are advantageously present in the oily phase.

### Definition of the Oily Phase:

As oils that may be used in the composition of the invention, mention may be made for example of:
hydrocarbon-based oils of animal origin, such as perhydrosqualene;
hydrocarbon-based oils of plant origin, such as liquid triglycerides of fatty acids of 4 to 10 carbon atoms, such as heptanoic or octanoic acid triglycerides or alternatively, for example, sunflower oil, corn oil, soybean oil, marrow oil, grapeseed oil, sesame seed oil, hazelnut oil, apricot oil, macadamia oil, arara oil, sunflower oil, castor oil, avocado oil, caprylic/capric acid triglycerides such as those sold by the company Stéarineries Dubois or those sold under the names Miglyol 810, 812 and 818 by the company Dynamit Nobel, jojoba oil or shea butter oil;
synthetic esters and ethers in particular of fatty acids, such as the oils of formulae R₁COOR₂ and R₁OR₂ in which R₁ represents a fatty acid residue containing from 8 to 29 carbon atoms and R₂ represents a branched or unbranched hydrocarbon-based chain containing from 3 to 30 carbon atoms, such as, for example, purcellin oil, isononyl isononanoate, isopropyl myristate, 2-ethylhexyl palmitate, 2-octyldodecyl stearate, 2-octyldodecyl erucate or isostearyl isostearate; hydroxylated esters such as isostearyl lactate, octyl hydroxystearate, octyldodecyl hydroxystearate, diisostearyl malate, triisocetyl citrate, and fatty alcohol heptanoates, octanoates and decanoates; polyol esters such as propylene glycol dioctanoate, neopentyl glycol diheptanoate and diethylene glycol diisononanoate; and pentaerythritol esters such as pentaerythrityl tetraisostearate;
linear or branched hydrocarbons of mineral or synthetic origin, such as volatile or non-volatile liquid paraffins and derivatives thereof, isohexadecane, isododecane, petroleum jelly, polydecenes or hydrogenated polyisobutene such as Parleam Oil®;
natural or synthetic essential oils such as, for example, eucalyptus oil, lavandin oil, lavender oil, vetiver oil, Litsea cubeba oil, lemon oil, sandalwood oil, rosemary oil, camomile oil, savory oil, nutmeg oil, cinnamon oil, hyssop oil, caraway oil, orange oil, geraniol oil, cade oil and bergamot oil;
fatty alcohols containing from 8 to 26 carbon atoms, such as cetyl alcohol, stearyl alcohol, and the mixture thereof (cetylstearyl alcohol), octyldodecanol, 2-butyloctanol, 2-hexyldecanol, 2-undecylpentadecanol, oleyl alcohol or linoleyl alcohol;
partially hydrocarbon-based and/or silicone-based fluoro oils such as those described in document JP-A-2-295 912;
silicone oils such as volatile or non-volatile polydimethylsiloxanes (PDMSs) containing a linear or cyclic silicone chain, which are liquid or pasty at room temperature, in particular cyclopolydimethyl-siloxanes (cyclomethicones) such as cyclohexasiloxane; polydimethylsiloxanes comprising alkyl, alkoxy or phenyl groups, pendant or at the end of a silicone chain, these groups containing from 2 to 24 carbon atoms; phenylsilicones such as phenyl trimethicones, phenyl dimethicones, phenyltrimethylsiloxydiphenyl-siloxanes, diphenyl dimethicones, diphenylmethyl-diphenyltrisiloxanes, 2-phenylethyl trimethylsiloxy-silicates and polymethylphenylsiloxanes;
mixtures thereof.

The term “hydrocarbon-based oil” in the list of oils mentioned above means any oil predominantly comprising carbon and hydrogen atoms, and optionally ester, ether, fluoro, carboxylic acid and/or alcohol groups.

The other fatty substances that may be present in the oily phase are, for example, fatty acids containing from 8 to 30 carbon atoms, for instance stearic acid, lauric acid, palmitic acid and oleic acid; waxes, for instance lanolin, beeswax, carnauba wax or candelilla wax, paraffin wax, lignite wax, microcrystalline wax, ceresin or ozokerite, synthetic waxes, for instance polyethylene waxes and Fischer-Tropsch waxes; gums such as silicone gums (dimethiconol); silicone resins such as trifluoromethyl-C1-4-alkyl dimethicone and trifluoropropyl dimethicone.

These fatty substances may be chosen in a varied manner by a person skilled in the art in order to prepare compositions having the desired properties, for example in terms of consistency or texture.

The compositions according to the invention may also comprise a volatile oil.

For the purposes of the invention, the term “volatile oil” means an oil that is capable of evaporating on contact with keratin materials in less than one hour, at room temperature and atmospheric pressure. The volatile organic solvent(s) and volatile oils of the invention are volatile organic solvents and volatile cosmetic oils that are liquid at room temperature, with a non-zero vapour pressure, at room temperature and atmospheric pressure, ranging in particular from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1300 Pa (0.01 to 10 mmHg).

Volatile oils that may be mentioned, inter alia, include cyclic or linear silicones containing from 2 to 6 silicon atoms, such as cyclohexasiloxane, dodecamethylpentasiloxane, decamethyltetrasiloxane, butyl trisiloxane and ethyl trisiloxane. It is also possible to use branched hydrocarbons, for instance isododecane, and also volatile perfluoroalkanes such as dodecafluoropentane and tetradecafluorohexane, sold under the names PF 5050® and PF 5060® by the company 3M, and perfluoro morpholine derivatives, such as 4-trifluoromethylperfluoromorpholine sold under the name PF 5052® by the company 3M.

The amount of oily phase present in the compositions according to the invention may range, for example, from 1% to 90% by weight and preferably from 5% to 70% by weight relative to the total weight of the composition under consideration.

The content of volatile oil present in the compositions according to the invention may be, for example, less than or equal to 50% by weight, preferably less than or equal to 30% by weight and better still less than or equal to 10% by weight relative to the total weight of the composition under consideration.

The emulsions generally contain at least one emulsifier chosen from amphoteric, anionic, cationic and nonionic emulsifiers, used alone or as a mixture. The emulsifiers are generally present in the composition in a proportion that may range, for example, from 0.3% to 30% by weight and preferably from 0.5% to 20% by weight relative to the total weight of the composition.

Needless to say, the emulsifying system is chosen so as to effectively stabilize the emulsions more particularly under consideration according to the invention. This choice falls within the competence of a person skilled in the art.

### Surfactants

Examples of emulsifiers that may be mentioned for the O/W emulsions include nonionic surfactants, and especially esters of polyols and of fatty acids with a saturated or unsaturated chain containing, for example, from 8 to 24 carbon atoms and better still from 12 to 22 carbon atoms, and the oxyalkylenated derivatives thereof, i.e. derivatives containing oxyethylenated and/or oxypropylenated units, such as the glyceryl esters of C₈-C₂₄ fatty acids, and the oxyalkylenated derivatives thereof; the polyethylene glycol esters of C₈-C₂₄ fatty acids, and the oxyalkylenated derivatives thereof; the sorbitol esters of C₈-C₂₄ fatty acids, and the oxyalkylenated derivatives thereof; the sugar (sucrose, glucose or alkylglucose) esters of C₈-C₂₄ fatty acids, and the oxyalkylenated derivatives thereof; fatty alkyl ethers; the sugar ethers of C₈-C₂₄ fatty alcohols, and mixtures thereof.

Glyceryl esters of fatty acids that may especially be mentioned include glyceryl stearate (glyceryl mono-, di- and/or tristearate) (CTFA name: glyceryl stearate) or glyceryl ricinoleate, and mixtures thereof.

Polyethylene glycol esters of fatty acids that may especially be mentioned include polyethylene glycol stearate (polyethylene glycol mono-, di- and/or tristearate) and more especially polyethylene glycol 50 OE monostearate (CTFA name: PEG-50 stearate) and polyethylene glycol 100 OE monostearate (CTFA name: PEG-100 stearate), and mixtures thereof.

Mixtures of these surfactants may also be used, for instance the product containing glyceryl stearate and PEG-100 stearate, sold under the name Arlacel 165 by the company Uniqema, and the product containing glyceryl stearate (glyceryl mono-distearate) and potassium stearate, sold under the name Tegin by the company Goldschmidt (CTFA name: glyceryl stearate SE).

Fatty acid esters of glucose or of alkylglucose that may be mentioned in particular include glucose palmitate, alkylglucose sesquistearates, for instance methylglucose sesquistearate, alkylglucose palmitates, for instance methylglucose palmitate or ethylglucose palmitate, fatty esters of methylglucoside and more especially the diester of methylglucoside and of oleic acid (CTFA name: Methyl glucose dioleate); the mixed ester of methylglucoside and of the oleic acid/hydroxystearic acid mixture (CTFA name: Methyl glucose dioleate/hydroxysterate); the ester of methylglucoside and of isostearic acid (CTFA name: Methyl glucose isostearate); the ester of methylglucoside and of lauric acid (CTFA name: Methyl glucose laurate); the mixture of the monoester and diester of methylglucoside and of isostearic acid (CTFA name: Methyl glucose sesquiisostearate); the mixture of the monoester and diester of methylglucoside and of stearic acid (CTFA name: Methyl glucose sesquistearate) and in particular the product sold under the name Glucate SS by the company Amerchol, and mixtures thereof.

Examples of oxyethylenated ethers of a fatty acid and of glucose or of alkylglucose that may be mentioned include the oxyethylenated ethers of a fatty acid and of methylglucose, and in particular the polyethylene glycol ether of the diester of methyl glucose and of stearic acid containing about 20 mol of ethylene oxide (CTFA name: PEG-20 methyl glucose distearate), such as the product sold under the name Glucam E-20 distearate by the company Amerchol; the polyethylene glycol ether of the mixture of monoester and diester of methylglucose and of stearic acid containing about 20 mol of ethylene oxide (CTFA name: PEG-20 methyl glucose sesquistearate) and in particular the product sold under the name Glucamate SSE-20 by the company Amerchol, and the product sold under the name Grillocose PSE-20 by the company Goldschmidt, and mixtures thereof.

Examples of sucrose esters that may be mentioned include sucrose palmitostearate, sucrose stearate and sucrose monolaurate.

Examples of fatty alkyl ethers that may be mentioned include polyethylene glycol ethers of fatty alcohols containing from 8 to 30 carbon atoms and especially from 10 to 22 carbon atoms, such as polyethylene glycol ethers of cetyl alcohol, of stearyl alcohol or of cetearyl alcohol (mixture of cetyl alcohol and stearyl alcohol). Examples that may be mentioned include ethers comprising from 1 to 200 and preferably from 2 to 100 oxyethylene groups, such as those of CTFA name Ceteareth-20 and Ceteareth-30, and mixtures thereof.

Sugar ethers that may especially be mentioned are alkylpolyglucosides, for example decylglucoside, for instance the product sold under the name Mydol 10 by the company Kao Chemicals, the product sold under the name Plantaren 2000 by the company Cognis, and the product sold under the name Oramix NS 10 by the company SEPPIC; caprylyl/capryl glucoside, for instance the product sold under the name Oramix CG 110 by the company SEPPIC or under the name Lutensol GD 70 by the company BASF; laurylglucoside, for instance the products sold under the names Plantaren 1200 N and Plantacare 1200 by the company Cognis; cocoglucoside, for instance the product sold under the name Plantacare 818/UP by the company Cognis; cetostearyl glucoside optionally as a mixture with cetostearyl alcohol, sold, for example, under the name Montanov 68 by the company SEPPIC, under the name Tego-Care CG90 by the company Goldschmidt and under the name Emulgade KE3302 by the company Cognis; arachidyl glucoside, for example in the form of the mixture of arachidyl alcohol and behenyl alcohol and arachidyl glucoside, sold under the name Montanov 202 by the company SEPPIC; cocoylethylglucoside, for example in the form of the mixture (35/65) with cetyl alcohol and stearyl alcohol, sold under the name Montanov 82 by the company SEPPIC; and mixtures thereof.

Emulsifying silicone elastomers may also be used, such as those sold under the names KSG-210, KSG-310, KSG-320, KSG-330, KSG-440, KSG-710, KSG-830 and KSG-840 by the company Shin-Etsu.

Examples of emulsifiers that may be mentioned for the W/O emulsions include dimethicone copolyols, such as the mixture of cyclomethicone and of dimethicone copolyol, sold under the name DC 5225 C by the company Dow Corning, and alkyldimethicone copolyols, such as the laurylmethicone copolyol sold under the name Dow Corning 5200 Formulation Aid by the company Dow Corning, the cetyldimethicone copolyol sold under the name Abil EM 90 by the company Goldschmidt, or the mixture of cetyldimethicone copolyol, polyglyceryl-4 isostearate and hexyl laurate, sold under the name Abil WE 09 by the company Goldschmidt. One or more co-emulsifiers may also be added thereto, which may be advantageously chosen from the group comprising alkylated esters of polyol. Alkylated esters of polyol that may especially be mentioned include glycerol and/or sorbitan esters, for example polyglyceryl isostearate, such as the product sold under the name Isolan GI 34 by the company Goldschmidt, sorbitan isostearate, such as the product sold under the name Arlacel 987 by the company ICI, sorbitan glyceryl isostearate, such as the product sold under the name Arlacel 986 by the company ICI, and mixtures thereof.

A crosslinked elastomeric solid organopolysiloxane comprising at least one oxyalkylene group may also be used as emulsifier for W/O emulsions, such as those obtained according to the procedure of Examples 3, 4 and 8 of document US-A-5 412 004 and of the examples of document US-A-5 811 487, especially the product of Example 3 (synthesis Example) of patent US-A-5 412 004 and such as the product sold under the reference KSG 21 by the company Shin-Etsu.

As emulsifiers suitable for obtaining a W/O emulsion, polyisobutylene surfactants containing esterified succinic end groups are especially suitable for use, such as those sold under the names Lubrizol 5603® and Chemcinnate 2000® by the companies Lubrizol and Chemron.

The compositions according to the invention may also comprise at least one dyestuff chosen, for example, from pigments, nacres, dyes and materials with an effect, and mixtures thereof.

These dyestuffs may be present in a content ranging from 0.01% to 50% by weight and preferably from 0.01% to 30% by weight relative to the total weight of the composition.

The compositions according to the invention may comprise a filler especially in a content ranging from 0.01% to 50% by weight and preferably ranging from 0.01% to 30% by weight relative to the total weight of the composition. These fillers may be mineral or organic and of any form, platelet-shaped, spherical or oblong, irrespective of the crystallographic form (for example lamellar, cubic, hexagonal, orthorhombic, etc.). Mention may be made of talc, mica, kaolin, lauroyllysine, starch, boron nitride, barium sulfate, precipitated calcium sulfate, magnesium carbonate, magnesium hydrogen carbonate, hydroxyapatite, glass or ceramic microcapsules, and metal soaps derived from organic carboxylic acids containing from 8 to 22 carbon atoms and preferably from 12 to 18 carbon atoms, for example zinc stearate, magnesium stearate or lithium stearate, zinc laurate or magnesium myristate.

The composition according to the invention may also contain various adjuvants commonly used in cosmetics, such as sequestrants; fragrances; and thickeners and gelling agents. The amounts of these various adjuvants and the nature thereof will be chosen such that they do not harm the optical properties of the composition.

Depending on the fluidity of the composition that it is desired to obtain, one or more gelling agents, especially hydrophilic gelling agents, i.e. water-soluble or water-dispersible gelling agents, may be incorporated into the composition.

Hydrophilic gelling agents that may be mentioned in particular include water-soluble or water-dispersible thickening polymers. These polymers may be chosen especially from: modified or unmodified carboxyvinyl polymers, such as the products sold under the names Carbopol (CTFA name: carbomer) by the company Goodrich; polyacrylates and polymethacrylates such as the products sold under the names Lubrajel and Norgel by the company Guardian or under the name Hispagel by the company Hispano Chimica; polyacrylamides; optionally crosslinked and/or neutralized 2-acrylamido-2-methylpropane sulfonic acid polymers and copolymers, for instance the poly(2-acrylamido-2-methylpropane-sulfonic acid) sold by the company Clariant under the name “Hostacerin AMPS” (CTFA name: ammonium polyacryldimethyltauramide); crosslinked anionic copolymers of acrylamide and of AMPS, which are in the form of a W/O emulsion, such as those sold under the name Sepigel 305 (CTFA name: Polyacrylamide/C13-14 Isoparaffin/Laureth-7) and under the name Simulgel 600 (CTFA name: Acrylamide/Sodium acryloyldimethyltaurate copolymer/Isohexadecane/Polysorbate 80) by the company SEPPIC; polysaccharide biopolymers, for instance xanthan gum, guar gum, carob gum, acacia gum, scleroglucans, chitin and chitosan derivatives, carrageenans, gellans, alginates, celluloses such as microcrystalline cellulose, carboxymethylcellulose, hydroxymethylcellulose and hydroxypropylcellulose; and mixtures thereof.

Examples of lipophilic gelling agents that may be mentioned include modified clays such as modified magnesium silicate (Bentone Gel VS38 from Rheox), or hectorite modified with distearyldimethylammonium chloride (CTFA name: Disteardimonium hectorite) sold under the name Bentone 38 CE by the company Rheox.

Examples of lipophilic gelling agents that may also be mentioned include modified clays such as modified magnesium silicate (Bentone gel VS38 from Rheox), hectorite modified with distearyldimethylammonium chloride (CTFA name: distearate diammonium hectorite) sold under the name Bentone 38 CE by the company Rheox, or silicone elastomers such as those sold under the names KSG-6 or KSG-16 by the company Shin-Etsu.

According to one preferred embodiment of the invention, at least one of the compositions of the kit according to the invention contains at least one UV-screening agent (or sunscreen), which may be a chemical screening agent or a physical screening agent, or a mixture of such screening agents.

Non-limiting illustrations that may be mentioned include the following families (the names correspond to the CTFA nomenclature of screening agents): anthranilates, in particular menthyl anthranilate; benzophenones, in particular benzophenone-1, benzophenone-3, benzophenone-5, benzophenone-6, benzophenone-8, benzophenone-9, benzophenone-12 and, preferentially, benzophenone-2 (oxybenzone) or benzophenone-4 (Uvinul MS40 available from BASF); benzylidenecamphors, in particular 3-benzylidene-camphor, benzylidenecamphorsulfonic acid, camphor benz-alkonium methosulfate, polyacrylamidomethylbenzyl-idenecamphor, terephthalylidenedicamphorsulfonic acid and, preferentially, 4-methylbenzylidenecamphor (Eusolex 6300 available from Merck); benzimidazoles, in particular benzimidazilate (Neo Heliopan AP available from Haarmann & Reimer), or phenylbenzimidazolesulfonic acid (Eusolex 232 available from Merck); benzotriazoles, in particular drometrizole trisiloxane, or methylenebis-benzotriazolyltetramethylbutylphenol (Tinosorb M available from Ciba); cinnamates, in particular cinoxate, DEA methoxycinnamate, diisopropyl methylcinnamate, glyceryl ethylhexanoate dimethoxy-cinnamate, isopropyl methoxycinnamate, isoamyl cinnamate and, preferentially, ethocrylene (Uvinul N35 available from BASF), octyl methoxycinnamate (Parsol MCX available from Hoffmann La Roche), or octocrylene (Uvinul 539 available from BASF); dibenzoylmethanes, in particular butylmethoxydibenzoylmethane (Parsol 1789); imidazolines, in particular ethylhexyl dimethoxy-benzylidene dioxoimidazoline; PABAS, in particular ethyl dihydroxypropyl PABA, ethylhexyldimethyl PABA, glyceryl PABA, PABA, PEG-25 PABA and, preferentially, diethylhexylbutamidotriazone (Uvasorb HEB available from 3V Sigma), ethylhexyltriazone (Uvinul T150 available from BASF) or ethyl PABA (benzocaine); salicylates, in particular dipropylene glycol salicylate, ethylhexyl salicylate, homosalate or TEA salicylate; triazines, in particular anisotriazine (Tinosorb S available from Ciba); drometrizole trisiloxane, zinc oxide, titanium dioxide, zinc oxide, iron oxide, zirconium oxide or cerium oxide, which may be coated or uncoated.

The amount of screening agents depends on the intended final use. It may range, for example, from 1% to 20% by weight and better still from 2% to 10% by weight relative to the total weight of the composition.

The products and compositions according to the invention may be in the form of a care or makeup product for the face and/or the body, and may be conditioned, for example in the form of cream in a jar or of fluid in a tube or in a pump-dispenser bottle.

They may advantageously be used for caring for and/or making up the skin, the lips, keratin fibres and in particular the eyelashes and/or the nails, depending on the nature of the ingredients used.

According to one embodiment, the compositions are compositions for making up and/or caring for the lips.

According to another embodiment, the compositions are compositions for coating the eyelashes or the eyebrows and more particularly mascaras.

According to another embodiment, the compositions are compositions for coating bodily or facial skin, more particularly makeup or care compositions for bodily or facial skin, for instance foundations or body makeup compositions.

A person skilled in the art may select the appropriate galenical form, and also the method for preparing it, on the basis of his general knowledge, taking into account firstly the nature of the constituents used, especially their solubility in the support, and secondly the intended use of each composition.

The cosmetic compositions according to the invention are especially intended for improving the aesthetic appearance of the skin and/or its integuments, in particular for improving the surface appearance and/or the texture of the skin.

The term “surface appearance” especially means the visual and/or tactile irregularities of the skin and/or the scalp, in particular wrinkles and fine lines, expression wrinkles, in particular on the forehead and between the eyebrows, wrinkles and fine lines around the mouth, and/or slackening around the lips, in particular in the top lip area (area between the top lip and the nose), the heterogeneity of the complexion (age marks, actinic lentigo), the appearance and/or visibility of the pores, the weathered appearance of the skin, aesthetic pigmentation defects (brownish spots), the visibility and/or size of the pores, skin microrelief defects such as chicken pox or acne marks, and imperfections of greasy skin (shiny appearance).

The term “texture of the skin” especially means skin that is soft, flaccid, less firm and less elastic, or slackened skin.

Ageing of the contour of the lips, in particular of the top lip area, is characteristic of women and especially of menopausal women, associated with an age-related hormonal deficit. This ageing is reflected especially by lengthening of the top lip area (increase in its height), by the appearance of vertical fine lines, by hollowing of the nasal groove and a reduction of the fat mass of the cheeks.

Expression wrinkles are produced by the effect of the stress exerted on the skin by the skin muscles that enable expressions. Depending on the shape of the face, the frequency of expressions and any possible tics, they may appear as early as childhood. Age, and even certain environmental factors such as exposure to sunlight, does not play a part in creating them, but may make them deeper and permanent. Expression wrinkles are characterized by the presence of grooves in the area around the orifices of the nose (nasal grooves), the mouth (perioral wrinkles and “bitterness” wrinkles) and the eyes (crow's feet wrinkles), around which lie the skin muscles, and also between the eyebrows (glabella or lion wrinkles) and on the forehead. In particular, it will be sought to prevent and/or smooth out the wrinkles of the forehead and of the area between the eyebrows.

The appearance and/or visibility of the pores is a quite recent problem, which it is sought to treat in particular in the case of individuals with dilated pores, irrespective of their origin: ethnic (e.g.: Asiatic population, Caucasian populations), excess sebum, ageing, loss of firmness, slackening, stress, fatigue, unsuitable hygiene, climatic factors, etc.

This skin imperfection, in particular on the face and especially of the T area (forehead, nose, cheeks and chin), in particular of the nose and the cheeks, may be accentuated by an increase in the width of the conical portion around the pore and/or a parakeratotic state of the stratum corneum in this conical portion, which is itself associated with excessive secretion of sebum and of unsaturated fatty acids. Agents that are active on the pores improve the appearance and/or visibility of the pores, in particular tightening thereof, and thus reduce the size of dilated pores, therefore making them less visible; the grain of the skin is thus tightened and refined, to give a smoother skin surface appearance and feel and a refined grain; the skin is more radiant and/or transparent.

The weathered appearance of the skin is characterized by a change in the visual appearance, and also in the behaviour of the skin to the touch. More specifically, the skin visually takes on the appearance of cigarette paper, giving it an appearance similar to that of a sheet of papyrus. In addition, when it is pinched gently between the thumb and the index finger, the skin forms numerous fine, sharp folds having the appearance of folded paper. Finally, the feel of the skin shows that its surface parts appear to be floating on the deeper parts, giving the skin, in the very advanced stage of the weathered appearance, the appearance of crumpled paper. The weathered appearance of the skin is visible on the face and even more characteristic on the back of the hands of the elderly.

The compositions according to the invention comprising at least one dermatological active agent according to the invention are especially intended for treating skin disorders associated with an impairment in the desquamation and/or pigmentation of the skin.

The desquamation disorders especially include: xerosis, acne, hyperkeratosis, psoriasis, atopy, ichthyosis.

Pigmentation disorders that may especially be mentioned include: melasma of the forearms, idiopathic melasma, hyperpigmentations associated with pregnancy or with combination oral contraception, puva-senile lentigo, accidental hyperpigmentations, leucodermia-related hyperpigmentations and vitiligo.

In particular, the process according to the invention is directed towards promoting the bleaching and/or the pigmentation of the skin and/or homogenizing the complexion and/or smoothing out the microrelief (attenuating wrinkles and fine lines), and/or preventing or combating the signs of ageing of the skin.

According to another embodiment, the process is directed towards attenuating cutaneous imperfections of greasy skin, and in particular towards making the skin mat.

According to the invention, the expression “cutaneous imperfections of greasy skin” especially means aesthetic disorders such as shiny skin, poorer staying power of makeup, a thick skin grain generally associated with a desquamation defect, accentuated visibility of the pores, a skin whose follicular orifices are dilated or filled with tiny horny spicules, or even with comedones or blackheads (which results, however, more from a phenomenon of retention than from an increase in excretion).

The term “matting” means making the skin visibly more mat and less shiny. The matting effect of the composition may especially be evaluated using a gonioreflectometer, by measuring the ratio R between the specular reflection and the diffuse reflection. A value of R of less than or equal to 2 generally reflects a matting effect.

In particular, the composition is applied to the areas of the face or of the forehead that have shiny skin.

According to another application of the invention, the process is directed towards attenuating cutaneous imperfections associated with ageing, in particular with actinic ageing.

The expression “cutaneous imperfections associated with ageing of the skin”, in particular actinic ageing, especially means a loss of firmness and/or elasticity and/or tonicity and/or suppleness of the skin, the formation of wrinkles and fine lines, expression wrinkles, in particular on the forehead and between the eyebrows, wrinkles and fine lines around the mouth, and/or slackening in the area around the lips, in particular in the top lip area (area located between the top lip and the nose), a dull appearance of the complexion, the appearance of browning and/or yellowing of the skin, and/or the appearance of senescence marks or age marks, heterogeneity of the complexion (age marks or actinic lentigo), the appearance and/or visibility of the pores, and the weathered appearance of the skin.

The process will especially be intended for individuals with mature or even very mature skin.

According to the invention, the term “mature skin” especially refers to the skin of people at least 40 years old.

According to the invention, the term “very mature skin” especially refers to the skin of people at least 50, in particular at least 60 or even 65 years old.

The compositions according to the invention intended for preventing and/or smoothing out expression wrinkles will be applied to the area around the orifices of the nose (nasal grooves), the mouth (perioral wrinkles and “bitterness” wrinkles) and the eyes (crow's feet wrinkles), around which lie the skin muscles, and also between the eyebrows (glabella or lion wrinkles) and on the forehead. In particular, it will be sought to prevent and/or smooth out the wrinkles on the forehead and between the eyebrows.

The compositions according to the invention for preventing and/or treating ageing of the area around the lips will in particular be applied to menopausal women, in particular to the top lip area.

The compositions according to the invention for preventing and/or treating the weathered appearance of the skin will be applied especially to the back of the hands.

The compositions according to the invention for reducing the appearance and/or visibility of the pores will be applied in particular to the T area (forehead, nose, cheeks and chin) and especially in the case of Asiatic or Caucasian populations.

Thus, the kit and/or the process according to the invention will be intended especially for treating at least one of the following conditions:
improving the radiance and/or homogeneity of the complexion and/or reducing the cloudy and/or dull appearance of the complexion;
improving the surface appearance, in particular reducing the rough or cracked appearance of the skin and/or improving the grain and/or softness of the skin;
smoothing out the skin microrelief, in particular smoothing out wrinkles and fine lines and/or attenuating acne or chicken pox marks;
attenuating age marks and/or aesthetic pigmentation disorders;
reducing the dryness of the skin, in particular age-related dryness;
improving the appearance and/or reducing the visibility of the pores and/or improving the unblocking of the pores;
promoting the cleansing action and the removal of dead cells at the surface of the skin; and/or
combating the imperfections of greasy skin, in particular the shiny or lustrous appearance of the skin.

According to a first mode, the process is intended for improving the radiance and/or homogeneity of the complexion and/or for reducing the cloudy and/or dull appearance of the complexion.

According to another mode, the process is intended for improving the surface appearance, in particular for reducing the rough or cracked appearance of the skin and/or for improving the grain and/or softness of the skin.

According to yet another mode, the process is intended for smoothing out the skin microrelief, in particular for smoothing out wrinkles and fine lines and/or attenuating acne or chicken pox marks.

According to yet another mode, the process is intended for attenuating age marks and/or aesthetic pigmentation disorders.

According to yet another mode, the process is intended for reducing the dryness of the skin, in particular age-related dryness.

According to yet another mode, the process is intended for improving the appearance and/or reducing the visibility of the pores and/or improving the unblocking of the pores.

According to yet another mode, the process is intended for promoting the cleansing action and removing dead cells from the surface of the skin.

According to yet another mode, the process is intended for combating the imperfections of greasy skin, in particular the shiny or lustrous appearance of the skin.

According to one particular embodiment of the invention, the compositions according to the invention may be combined with orally administered compositions, containing additional cosmetic active agents with a beneficial effect on the appearance of the skin, for instance additional active agents for combating the signs of ageing of the skin or additional active agents for combating greasy skin.

The above written description of the invention provides a manner and process of making and using it such that any person skilled in this art is enabled to make and use the same, this enablement being provided in particular for the subject matter of the appended claims, which make up a part of the original description and including a cosmetic and/or dermatological kit for making up or non-therapeutically caring for keratin material(s), comprising
at least one cosmetic and/or dermatological active agent for improving the appearance of the keratin materials,
at least two different compositions conditioned separately, the kit comprising at least one compound X and at least one compound Y, and optionally at least one catalyst or one peroxide, with at least one of the compounds X and Y being a silicone compound, and the compounds X and Y being capable of reacting together via a hydrosilylation reaction, or via a condensation reaction, or via a crosslinking reaction in the presence of a peroxide, when they are placed in contact with each other,
   the cosmetic and/or dermatological active agent(s) being present in at least one of the two first compositions, or in a third composition separate from the first two.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted. Phrases such as “mention may be made,” etc. preface examples of materials that can be used and do not limit the invention to the specific materials, etc., listed.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. Cosmetic and/or dermatological kit for making up or non-therapeutically caring for keratin material(s), comprising
at least 0.01% by weight relative to the total weight of the composition of a moisturizer agent for improving the appearance of the keratin materials,
at least two different compositions conditioned separately, the kit comprising at least one compound X and at least one compound Y, and at least one catalyst, with at least one of the compounds X and Y being a silicone compound, and the compounds X and Y being capable of reacting together via a hydrosilylation reaction in the presence of a catalyst, when they are placed in contact with each other,
the moisturizer agent(s) being present in at least one of the two first compositions, or in a third composition separate from the first two.

2. Kit according to claim 1, wherein at least one of the compositions containing the compound X or the compound Y of the kit is in the form of a simple or multiple W/O/W emulsion.

3. Kit according to claim 1, comprising:
at least
i. a first composition comprising, in a physiologically acceptable medium, at least one compound X, and
ii. a second composition comprising, in a physiologically acceptable medium, at least one compound Y, and
at least one moisturizer agent, the active agent(s) being present in at least one of the first or second compositions, or in a third composition separate from the first two.

4. Kit according to claim 1, comprising:
at least:
i. a first composition comprising, in a physiologically acceptable medium, at least one compound X and one compound Y, the compounds X and Y being capable of reacting together in the presence of a catalyst via a hydrosilylation reaction, when they are placed in contact with each other, and
ii. a second composition comprising, in a physiologically acceptable medium, at least the catalyst required for the interaction of compounds X and Y,
at least one moisturizer agent as defined below, the active agent(s) being present in at least one of the first or second compositions, or in a third composition separate from the first two.

5. Kit according to claim 4, wherein compound X is chosen from silicone compounds comprising at least two unsaturated aliphatic groups.

6. Kit according to claim 5, in which compound X is a polyorganosiloxane comprising a silicone main chain whose unsaturated aliphatic groups are pendent on the main chain (side group) or located at the ends of the main chain of the compound (end group).

7. Kit according to claim 6, wherein compound X bears at least one polar group.

8. Kit according to claim 1, wherein compound X is chosen from polyorganosiloxanes comprising at least two unsaturated aliphatic groups each linked to a silicon atom.

9. Kit according to claim 1, wherein compound X is chosen from polyorganosiloxanes comprising siloxane units of formula:
$${R}_{m} {R}^{′} {SiO}_{\frac{( 3 - m )}{2}} \qquad ( I )$$
in which:
R represents a linear or cyclic monovalent hydro-carbon-based group containing from 1 to 30 carbon atoms,
m is equal to 1 or 2, and
R′ represents:
an unsaturated aliphatic hydrocarbon-based group containing from 2 to 10 carbon atoms, or
an unsaturated cyclic hydrocarbon-based group containing from 5 to 8 carbon atoms.

10. Kit according to claim 9, in which the polyorganosiloxane of formula (I) is such that R′ represents a vinyl group or a group —R″-CH═CHR′″ in which R″ is a divalent aliphatic hydrocarbon-based chain containing from 1 to 8 carbon atoms, linked to the silicon atom, and R′″ is a hydrogen atom or an alkyl radical containing from 1 to 4 carbon atoms.

11. Kit according to claim 11, wherein R represents an alkyl radical containing from 1 to 10 carbon atoms or a phenyl group, and R′ is a vinyl group.

12. Composition according to claim 6, wherein the polyorganosiloxanes also comprise units of formula:
$${R}_{n} {SiO}_{\frac{( 4 - n )}{2}} \qquad ( II )$$
in which R represents a linear or cyclic monovalent hydrocarbon-based group containing from 1 to 30 carbon atoms, and n is equal to 1, 2 or 3.

13. Kit according to claim 1, wherein compound X is chosen from organic oligomers or polymers, organic/silicone hybrid oligomers or polymers, the oligomers or polymers bearing at least two reactive unsaturated aliphatic groups, and mixtures thereof.

14. Kit according to one of claims 1, wherein compound Y is chosen from organosiloxanes comprising at least two free Si—H groups.

15. Kit according to one of claims 1, wherein compound Y is chosen from polyorganosiloxanes comprising at least one alkylhydrogenosiloxane units having the following formula:
$${R}_{p} {HSiO}_{\frac{( 3 - p )}{2}} \qquad ( III )$$
in which:
R represents a linear or cyclic monovalent hydrocarbon-based group containing from 1 to 30 carbon atoms or a phenyl group, and p is equal to 1 or 2.

16. Kit according to claim 15, in which compound Y is such that the radicals R represents a C₁-C₁₀ alkyl group.

17. Kit according to claim 14, in which the organosiloxanes Y comprise at least two alkylhydrogenosiloxane units of formula —(H₃C)(H)Si—O— and optionally comprise —(H₃C)₂SiO—.

18. Kit according to claim 1, in which the catalyst is a platinum-based or tin-based catalyst.

19. Kit according to claim 18, wherein the catalyst represents from 0.0001% to 20% by weight relative to the total weight of the composition comprising it.

20. Kit according to claim 1, wherein compound X is a polydimethylsiloxane containing vinyl end groups and compound Y is a polymethylhydrogenosiloxane.

21. Kit according to claim 1, in which compound X bears at least one polar group capable of forming a hydrogen bond with keratin materials.

22. Kit according to claim 1, comprising, in at least one of the compositions, a filler chosen from silica and surface-treated silica.

23. Kit according to claim 1, wherein compound X has a weight-average molecular mass (Mw) ranging from 150 to 1 000 000.

24. Kit according to claim 1, wherein compound Y has a weight-average molecular mass (Mw) ranging from 200 to 1 000 000.

25. Kit according to claim 1, wherein compound X represents from 0.1% to 95% by weight relative to the total weight of the composition containing it.

26. Kit according to claim 1, wherein compound Y represents from 0.1% to 95% by weight relative to the total weight of the composition containing it.

27. Composition according to claim 1, wherein compounds X and Y are present in the compositions in a mole ratio X/Y ranging from 0.05 to 20.

28. Kit according to claim 1, in which the moisturizer agent is chosen from urea and derivatives thereof; hyaluronic acid, AHAs, BHAs, acrylic acid homopolymers, beta-glucan; a mixture of passionflower oil, apricot oil, corn oil and rice bran oil; a C-glycoside derivative and in particular C-β-D-xylopyranoside-2-hydroxypropane in the form of a solution containing 30% by weight of active material in a water/propylene glycol mixture (60/40% by weight); an oil of musk rose; an oil of the microalga Prophyridium cruentum enriched with zinc; spheres of collagen and of chondroitin sulfate of marine of origin (Atelocollagen); hyaluronic acid spheres; arginine; xylitol derivatives, as the mixture of xylitol-polyglucoside/xylitan/xylitol; the phytantriol or TETRA-METHYL-3,7,11,15 TRI-HYDROXY-1,2,3 HEXA-DECANE sold by the company DSM; and their mixtures.

29. Kit according to claim 28, in which the moisturizer agent is chosen from urea and its derivatives.

30. Kit according to claim 29, in which the moisturizer agent is chosen from xylitol and its derivatives.

31. Kit according to claim 20, in which the moisturizer agent is chosen from phytantriol [TETRA-METHYL-3,7,11,15 TRI-HYDROXY-1,2,3 HEXA-DECANE].

32. Kit according to claim 1, in which the moisturizer agent is present in at least one of the compositions in an amount from 0.01 to 20% in weight relative to the total weight of the composition.

33. Kit according to claim 32, wherein it further comprises at least one additional ingredient chosen from matting agents, fillers with a soft-focus effect, fluorescers, agents for promoting the naturally pinkish coloration of the skin, abrasive fillers or exfoliants, and mixtures thereof.

34. Kit according to claim 32, wherein it further comprises at least one UV-screening agent.

35. Kit according to claim 33, in which the compositions are conditioned separately in the same conditioning article.

36. Kit according to claim 1, for affording a film for coating bodily or facial skin.

37. Kit according to claim 1, for affording a film for coating the lips.

38. Kit according to claim 1, for affording a film for coating the nails.

39. Kit according to claim 1, for affording a film for coating keratin fibres.

40. A process for caring for or making up keratin material(s), comprising at least the application (a) of at least one moisturizer agent, (b) of one or more compounds X, (c) of one or more compounds Y with at least one of the compounds X and Y being a silicone compound and the compounds X and Y being capable of reacting together via a hydrosilylation reaction when they are placed in contact with each other, and, (d) of a catalyst for the interaction of the compound X with the compound Y, the applications of (a), (b), (c) and (d) possibly being simultaneous or sequential in any order, with the proviso that it is beneficial to the interaction of the compounds X and Y.

41. Process according to claim 40, which comprises applying to the keratin material(s) at least one composition comprising at least one compound X, at least one compound Y, at least one moisturizer agent and, at least one catalyst.

42. Process according to claim 41, in which the composition is obtained by mixing at the time of use at least a first composition comprising at least one compound X and a second composition comprising at least compound Y, at least one of the first and second compositions containing at least one catalyst and at least one moisturizer agent for improving the appearance of keratin materials.

43. A process for making up and/or caring for keratin material(s), comprising the application to the keratin material(s):
i. of at least one coat of a first composition comprising, in a physiologically acceptable medium, at least one compound X;
ii. of at least one coat of a second composition comprising, in a physiologically acceptable medium, at least one compound Y, at least one of the compounds X and Y being a silicone compound, the compounds X and Y being capable of reacting together via a hydrosilylation reaction in the presence of a catalyst, when they are placed in contact with each other, with at least one of the first and second compositions containing at least one moisturizer agent for improving the appearance of keratin materials.

44. Process according to claim 40, wherein it is intended for treating at least one condition chosen from:
improving the radiance and/or homogeneity of the complexion and/or reducing the cloudy and/or dull appearance of the complexion;
improving the surface appearance, in particular reducing the rough or cracked appearance of the skin and/or improving the grain and/or softness of the skin;
smoothing out the skin microrelief, in particular smoothing out wrinkles and fine lines and/or attenuating acne or chicken pox marks;
attenuating age marks and/or aesthetic pigmentation disorders;
reducing the dryness of the skin, in particular age-related dryness;
improving the appearance and/or reducing the visibility of the pores and/or improving the unblocking of the pores;
promoting the cleansing action and the removal of dead cells at the surface of the skin; and/or
combating the imperfections of greasy skin, in particular the shiny or lustrous appearance of the skin.
